SEQUENCE LISTING (119 sequences)

SEQ ID NO: 1 - RNA, 22 nt, Homo sapiens
  misc_feature: hsa-miR-1
uggaauguaaagaaguauguau

SEQ ID NO: 2 - RNA, 22 nt, Homo sapiens
  misc_feature: hsa-miR-9* (miR-9-3p)
auaaagcuagauaaccgaaagu

SEQ ID NO: 3 - RNA, 23 nt, Homo sapiens
  misc_feature: hsa-miR-10b* (hsa-miR-10b-3p)
uacccuguagaaccgaauuugug

SEQ ID NO: 4 - RNA, 22 nt, Homo sapiens
  misc_feature: hsa-miR-16 (hsa-miR-16-5p)
uagcagcacguaaauauuggcg

SEQ ID NO: 5 - RNA, 23 nt, Homo sapiens
  misc_feature: hsa-miR-17 (hsa-miR-17-5p)
caaagugcuuacagugcagguag

SEQ ID NO: 6 - RNA, 23 nt, Homo sapiens
  misc_feature: hsa-miR-18a (hsa-miR-18a-5p)
uaaggugcaucuagugcagauag

SEQ ID NO: 7 - RNA, 23 nt, Homo sapiens
  misc_feature: hsa-miR-19a
ugugcaaaucuaugcaaaacuga

SEQ ID NO: 8 - RNA, 23 nt, Homo sapiens
  misc_feature: hsa-miR-19b (hsa-miR-19b-3p)
ugugcaaauccaugcaaaacuga

SEQ ID NO: 9 - RNA, 23 nt, Homo sapiens
  misc_feature: hsa-miR-20a (hsa-miR-20a-5p)
uaaagugcuuauagugcagguag

SEQ ID NO: 10 - RNA, 23 nt, Homo sapiens
  misc_feature: hsa-miR-20b (hsa-miR-20b-5p)
caaagugcucauagugcagguag

SEQ ID NO: 11 - RNA, 22 nt, Homo sapiens
  misc_feature: hsa-miR-21 (hsa-miR-21-5p)
uagcuuaucagacugauguuga

SEQ ID NO: 12 - RNA, 21 nt, Homo sapiens
  misc_feature: hsa-miR-23a (hsa-miR-23a-3p)
aucacauugccagggauuucc

SEQ ID NO: 13 - RNA, 21 nt, Homo sapiens
  misc_feature: hsa-miR-23b (hsa-miR-23b-3p)
aucacauugccagggauuacc

SEQ ID NO: 14 - RNA, 22 nt, Homo sapiens
  misc_feature: hsa-miR-24 (hsa-miR-24-3p)
uggcucaguucagcaggaacag

SEQ ID NO: 15 - RNA, 22 nt, Homo sapiens
  misc_feature: hsa-miR-24-2* (hsa-miR-24-2-5p)
ugccuacugagcugaaacacag

SEQ ID NO: 16 - RNA, 22 nt, Homo sapiens
  misc_feature: hsa-miR-25 (hsa-miR-25-3p)
cauugcacuugucucggucuga

SEQ ID NO: 17 - RNA, 22 nt, Homo sapiens
  misc_feature: hsa-miR-26a (hsa-miR-26a-5p)
uucaaguaauccaggauaggcu

SEQ ID NO: 18 - RNA, 21 nt, Homo sapiens
  misc_feature: hsa-miR-26b (hsa-miR-26b-5p)
uucaaguaauucaggauaggu

SEQ ID NO: 19 - RNA, 21 nt, Homo sapiens
  misc_feature: hsa-miR-27a (hsa-miR-27a-3p)
uucacaguggcuaaguuccgc

SEQ ID NO: 20 - RNA, 22 nt, Homo sapiens
  misc_feature: hsa-miR-29c (hsa-miR-29c)
uagcaccauuugaaaucgguua

SEQ ID NO: 21 - RNA, 22 nt, Homo sapiens
  misc_feature: hsa-miR-30a-5p
uguaaacauccucgacuggaag

SEQ ID NO: 22 - RNA, 22 nt, Homo sapiens
  misc_feature: hsa-miR-30b (hsa-miR-30b-5p)
uguaaacauccuacacucagcu

SEQ ID NO: 23 - RNA, 22 nt, Homo sapiens
  misc_feature: hsa-miR-30e-3p
cuuucagucggauguuuacagc

SEQ ID NO: 24 - RNA, 23 nt, Homo sapiens
  misc_feature: hsa-miR-30c (hsa-miR-30c-5p)
uguaaacauccuacacucucagc

SEQ ID NO: 25 - RNA, 22 nt, Homo sapiens
  misc_feature: hsa-miR-30d (hsa-miR-30d-5p)
uguaaacauccccgacuggaag

SEQ ID NO: 26 - RNA, 21 nt, Homo sapiens
  misc_feature: hsa-miR-31 (hsa-miR-31-5p)
aggcaagaugcuggcauagcu

SEQ ID NO: 27 - RNA, 22 nt, Homo sapiens
  misc_feature: hsa-miR-34a (hsa-miR-34a-5p)
uggcagugucuuagcugguugu

SEQ ID NO: 28 - RNA, 23 nt, Homo sapiens
  misc_feature: hsa-miR-93 (hsa-miR-93-5p)
caaagugcuguucgugcagguag

SEQ ID NO: 29 - RNA, 22 nt, Homo sapiens
  misc_feature: miR-99a (hsa-miR-99a-5p)
aacccguagauccgaucuugug

SEQ ID NO: 30 - RNA, 22 nt, Homo sapiens
  misc_feature: hsa-miR-99b (hsa-miR-99b-5p)
cacccguagaaccgaccuugcg

SEQ ID NO: 31 - RNA, 22 nt, Homo sapiens
  misc_feature: hsa-miR-100 (hsa-miR-100-5p)
aacccguagauccgaacuugug

SEQ ID NO: 32 - RNA, 23 nt, Homo sapiens
  misc_feature: hsa-miR-106a (hsa-miR-106a-5p)
aaaagugcuuacagugcagguag

SEQ ID NO: 33 - RNA, 22 nt, Homo sapiens
  misc_feature: hsa-miR-122 (hsa-miR-122-5p)
uggagugugacaaugguguuug

SEQ ID NO: 34 - RNA, 22 nt, Homo sapiens
  misc_feature: hsa-miR-126 (hsa-miR-126-3p)
ucguaccgugaguaauaaugcg

SEQ ID NO: 35 - RNA, 21 nt, Homo sapiens
  misc_feature: hsa-miR-126* (hsa-miR-126-5p)
cauuauuacuuuugguacgcg

SEQ ID NO: 36 - RNA, 21 nt, Homo sapiens
  misc_feature: hsa-miR-128
ucacagugaaccggucucuuu

SEQ ID NO: 37 - RNA, 23 nt, Homo sapiens
  misc_feature: hsa-miR-139-5p
ucuacagugcacgugucuccagu

SEQ ID NO: 38 - RNA, 21 nt, Homo sapiens
  misc_feature: hsa-miR-140-3p
uaccacaggguagaaccacgg

SEQ ID NO: 39 - RNA, 22 nt, Homo sapiens
  misc_feature: hsa-miR-140-5p
cagugguuuuacccuaugguag

SEQ ID NO: 40 - RNA, 21 nt, Homo sapiens
  misc_feature: hsa-miR-143 (hsa-miR-143-3p)
ugagaugaagcacuguagcuc

SEQ ID NO: 41 - RNA, 22 nt, Homo sapiens
  misc_feature: hsa-miR-144* (hsa-miR-144-5p)
ggauaucaucauauacuguaag

SEQ ID NO: 42 - RNA, 23 nt, Homo sapiens
  misc_feature: hsa-miR-145 (hsa-miR-145-5p)
guccaguuuucccaggaaucccu

SEQ ID NO: 43 - RNA, 22 nt, Homo sapiens
  misc_feature: hsa-miR-146a (hsa-mir-146a-5p)
ugagaacugaauuccauggguu

SEQ ID NO: 44 - RNA, 22 nt, Homo sapiens
  misc_feature: hsa-miR-146b-5p
ugagaacugaauuccauaggcu

SEQ ID NO: 45 - RNA, 22 nt, Homo sapiens
  misc_feature: hsa-miR-148a (hsa-miR-148a-3p)
ucagugcacuacagaacuuugu

SEQ ID NO: 46 - RNA, 22 nt, Homo sapiens
  misc_feature: hsa-miR-150 (hsa-miR-150-5p)
ucucccaacccuuguaccagug

SEQ ID NO: 47 - RNA, 21 nt, Homo sapiens
  misc_feature: hsa-miR-151-3p
cuagacugaagcuccuugagg

SEQ ID NO: 48 - RNA, 21 nt, Homo sapiens
  misc_feature: hsa-miR-151-5p
ucgaggagcucacagucuagu

SEQ ID NO: 49 - RNA, 21 nt, Homo sapiens
  misc_feature: hsa-miR-152
ucagugcaugacagaacuugg

SEQ ID NO: 50 - RNA, 23 nt, Homo sapiens
  misc_feature: hsa-miR-155 (hsa-miR-155-5p)
uuaaugcuaaucgugauaggggu

SEQ ID NO: 51 - RNA, 23 nt, Homo sapiens
  misc_feature: hsa-miR-181a (hsa-miR-181a-5p)
aacauucaacgcugucggugagu

SEQ ID NO: 52 - RNA, 23 nt, Homo sapiens
  misc_feature: hsa-miR-181b (hsa-miR-181b-5p)
aacauucauugcugucggugggu

SEQ ID NO: 53 - RNA, 22 nt, Homo sapiens
  misc_feature: hsa-miR-185 (hsa-miR-185-5p)
uggagagaaaggcaguuccuga

SEQ ID NO: 54 - RNA, 22 nt, Homo sapiens
  misc_feature: hsa-miR-186 (hsa-miR-186-5p)
caaagaauucuccuuuugggcu

SEQ ID NO: 55 - RNA, 22 nt, Homo sapiens
  misc_feature: hsa-miR-190 (hsa-miR-190a)
ugauauguuugauauauuaggu

SEQ ID NO: 56 - RNA, 23 nt, Homo sapiens
  misc_feature: hsa-miR-191 (hsa-miR-191-5p)
caacggaaucccaaaagcagcug

SEQ ID NO: 57 - RNA, 21 nt, Homo sapiens
  misc_feature: hsa-miR-195 (hsa-miR-195-5p)
uagcagcacagaaauauuggc

SEQ ID NO: 58 - RNA, 22 nt, Homo sapiens
  misc_feature: hsa-miR-196a (hsa-miR-196a-5p)
uagguaguuucauguuguuggg

SEQ ID NO: 59 - RNA, 22 nt, Homo sapiens
  misc_feature: hsa-miR-196b (hsa-miR-196-5p)
uagguaguuuccuguuguuggg

SEQ ID NO: 60 - RNA, 22 nt, Homo sapiens
  misc_feature: hsa-miR-197 (hsa-miR-197-3p)
uucaccaccuucuccacccagc

SEQ ID NO: 61 - RNA, 23 nt, Homo sapiens
  misc_feature: hsa-miR-199b-5p
cccaguguuuagacuaucuguuc

SEQ ID NO: 62 - RNA, 22 nt, Homo sapiens
  misc_feature: hsa-miR-210
cugugcgugugacagcggcuga

SEQ ID NO: 63 - RNA, 21 nt, Homo sapiens
  misc_feature: hsa-miR-212 (hsa-miR-212-3p)
uaacagucuccagucacggcc

SEQ ID NO: 64 - RNA, 22 nt, Homo sapiens
  misc_feature: hsa-miR-223 (hsa-miR-223-3p)
ugucaguuugucaaauacccca

SEQ ID NO: 65 - RNA, 22 nt, Homo sapiens
  misc_feature: hsa-miR-223* (hsa-miR-223-5p)
cguguauuugacaagcugaguu

SEQ ID NO: 66 - RNA, 22 nt, Homo sapiens
  misc_feature: hsa-miR-296-3p
gaggguuggguggaggcucucc

SEQ ID NO: 67 - RNA, 22 nt, Homo sapiens
  misc_feature: hsa-miR-320 (hsa-miR-320a)
aaaagcuggguugagagggcga

SEQ ID NO: 68 - RNA, 22 nt, Homo sapiens
  misc_feature: hsa-miR-320b
aaaagcuggguugagagggcaa

SEQ ID NO: 69 - RNA, 21 nt, Homo sapiens
  misc_feature: hsa-miR-323-3p
cacauuacacggucgaccucu

SEQ ID NO: 70 - RNA, 20 nt, Homo sapiens
  misc_feature: hsa-miR-324-3p
acugccccaggugcugcugg

SEQ ID NO: 71 - RNA, 20 nt, Homo sapiens
  misc_feature: hsa-miR-326
ccucugggcccuuccuccag

SEQ ID NO: 72 - RNA, 22 nt, Homo sapiens
  misc_feature: hsa-miR-328
cuggcccucucugcccuuccgu

SEQ ID NO: 73 - RNA, 21 nt, Homo sapiens
  misc_feature: hsa-miR-331-3p
gccccugggccuauccuagaa

SEQ ID NO: 74 - RNA, 22 nt, Homo sapiens
  misc_feature: hsa-miR-338-5p
aacaauauccuggugcugagug

SEQ ID NO: 75 - RNA, 22 nt, Homo sapiens
  misc_feature: hsa-miR-340 (hsa-miR-340-5p)
uuauaaagcaaugagacugauu

SEQ ID NO: 76 - RNA, 22 nt, Homo sapiens
  misc_feature: hsa-miR-345 (hsa-miR-345-5p)
gcugacuccuaguccagggcuc

SEQ ID NO: 77 - RNA, 22 nt, Homo sapiens
  misc_feature: hsa-miR-362-3p
aacacaccuauucaaggauuca

SEQ ID NO: 78 - RNA, 22 nt, Homo sapiens
  misc_feature: hsa-miR-370
gccugcugggguggaaccuggu

SEQ ID NO: 79 - RNA, 22 nt, Homo sapiens
  misc_feature: hsa-miR-374a (hsa-miR-374a-5p)
uuauaauacaaccugauaagug

SEQ ID NO: 80 - RNA, 22 nt, Homo sapiens
  misc_feature: hsa-miR-374b* (hsa-miR-374b-3p)
cuuagcagguuguauuaucauu

SEQ ID NO: 81 - RNA, 21 nt, Homo sapiens
  misc_feature: hsa-miR-376c (hsa-miR-376c-3p)
aacauagaggaaauuccacgu

SEQ ID NO: 82 - RNA, 23 nt, Homo sapiens
  misc_feature: hsa-miR-432 (hsa-miR-432-5p)
ucuuggaguaggucauugggugg

SEQ ID NO: 83 - RNA, 22 nt, Homo sapiens
  misc_feature: hsa-miR-450a (hsa-miR-450a-5p)
uuuugcgauguguuccuaauau

SEQ ID NO: 84 - RNA, 22 nt, Homo sapiens
  misc_feature: hsa-miR-450b-5p
uuuugcaauauguuccugaaua

SEQ ID NO: 85 - RNA, 22 nt, Homo sapiens
  misc_feature: hsa-miR-451 (hsa-miR-451a)
aaaccguuaccauuacugaguu

SEQ ID NO: 86 - RNA, 22 nt, Homo sapiens
  misc_feature: hsa-miR-483-5p
aagacgggaggaaagaagggag

SEQ ID NO: 87 - RNA, 22 nt, Homo sapiens
  misc_feature: hsa-miR-484
ucaggcucaguccccucccgau

SEQ ID NO: 88 - RNA, 22 nt, Homo sapiens
  misc_feature: hsa-miR-485-3p
gucauacacggcucuccucucu

SEQ ID NO: 89 - RNA, 22 nt, Homo sapiens
  misc_feature: hsa-miR-505 (hsa-miR-505-3p)
cgucaacacuugcugguuuccu

SEQ ID NO: 90 - RNA, 23 nt, Homo sapiens
  misc_feature: hsa-miR-508-3p
ugauuguagccuuuuggaguaga

SEQ ID NO: 91 - RNA, 21 nt, Homo sapiens
  misc_feature: hsa-miR-511
gugucuuuugcucugcaguca

SEQ ID NO: 92 - RNA, 21 nt, Homo sapiens
  misc_feature: hsa-miR-518d (hsa-miR-518d-3p)
caaagcgcuucccuuuggagc

SEQ ID NO: 93 - RNA, 22 nt, Homo sapiens
  misc_feature: hsa-miR-520d-3p
aaagugcuucucuuuggugggu

SEQ ID NO: 94 - RNA, 22 nt, Homo sapiens
  misc_feature: hsa-miR-539 (hsa-miR-539-5p)
ggagaaauuauccuuggugugu

SEQ ID NO: 95 - RNA, 22 nt, Homo sapiens
  misc_feature: hsa-miR-548a (hsa-miR-548a-3p)
caaaacuggcaauuacuuuugc

SEQ ID NO: 96 - RNA, 22 nt, Homo sapiens
  misc_feature: hsa-miR-575
cacgcucaugcacacacccaca

SEQ ID NO: 97 - RNA, 19 nt, Homo sapiens
  misc_feature: hsa-miR-575
gagccaguuggacaggagc

SEQ ID NO: 98 - RNA, 22 nt, Homo sapiens
  misc_feature: hsa-miR-582-3p
uaacugguugaacaacugaacc

SEQ ID NO: 99 - RNA, 22 nt, Homo sapiens
  misc_feature: hsa-miR-590-5p
gagcuuauucauaaaagugcag

SEQ ID NO: 100 - RNA, 23 nt, Homo sapiens
  misc_feature: hsa-miR-618
aaacucuacuuguccuucugagu

SEQ ID NO: 101 - RNA, 21 nt, Homo sapiens
  misc_feature: hsa-miR-625 (hsa-miR-625-5p)
agggggaaaguucuauagucc

SEQ ID NO: 102 - RNA, 21 nt, Homo sapiens
  misc_feature: hsa-miR-628-3p
ucuaguaagaguggcagucga

SEQ ID NO: 103 - RNA, 23 nt, Homo sapiens
  misc_feature: hsa-miR-636
ugugcuugcucgucccgcccgca

SEQ ID NO: 104 - RNA, 25 nt, Homo sapiens
  misc_feature: hsa-miR-638
agggaucgcgggcggguggcggccu

SEQ ID NO: 105 - RNA, 19 nt, Homo sapiens
  misc_feature: hsa-miR-645
ucuaggcugguacugcuga

SEQ ID NO: 106 - RNA, 21 nt, Homo sapiens
  misc_feature: hsa-miR-652 (hsa-miR-652-3p)
aauggcgccacuaggguugug

SEQ ID NO: 107 - RNA, 22 nt, Homo sapiens
  misc_feature: hsa-miR-758 (hsa-miR-758-3p)
uuugugaccugguccacuaacc

SEQ ID NO: 108 - RNA, 22 nt, Homo sapiens
  misc_feature: hsa-miR-769-5p
ugagaccucuggguucugagcu

SEQ ID NO: 109 - RNA, 21 nt, Homo sapiens
  misc_feature: hsa-miR-875-3p
ccuggaaacacugagguugug

SEQ ID NO: 110 - RNA, 22 nt, Homo sapiens
  misc_feature: hsa-miR-874
cugcccuggcccgagggaccga

SEQ ID NO: 111 - RNA, 22 nt, Homo sapiens
  misc_feature: hsa-miR-875-3p
uccauuacacuacccugccucu

SEQ ID NO: 112 - RNA, 23 nt, Homo sapiens
  misc_feature: hsa-miR-935
ccaguuaccgcuuccgcuaccgc

SEQ ID NO: 113 - RNA, 23 nt, Homo sapiens
  misc_feature: hsa-miR-941
cacccggcugugugcacaugugc

SEQ ID NO: 114 - RNA, 22 nt, Homo sapiens
  misc_feature: hsa-miR-1225-3p
ugagccccugugccgcccccag

SEQ ID NO: 115 - RNA, 20 nt, Homo sapiens
  misc_feature: hsa-miR-1238 (hsa-miR-1238-3p)
cuuccucgucugucugcccc

SEQ ID NO: 116 - RNA, 18 nt, Homo sapiens
  misc_feature: hsa-miR-1260 (hsa-miR-1260a)
aucccaccucugccacca

SEQ ID NO: 117 - RNA, 22 nt, Homo sapiens
  misc_feature: hsa-miR-1305
uuuucaacucuaaugggagaga

SEQ ID NO: 118 - RNA, 22 nt, Homo sapiens
  misc_feature: hsa-let-7b (hsa-let-7b-5p)
ugagguaguagguugugugguu

SEQ ID NO: 119 - RNA, 22 nt, Homo sapiens
  misc_feature: hsa-let-7g (hsa-let-7g-5p)
ugagguaguaguuuguacaguu

(19) United States
(12) Patent Application Publication
(10) Pub. No.: US 2015/0011414 A1
(43) Pub. Date: Jan. 8, 2015

(54) MICRORNA FOR DIAGNOSIS OF PANCREATIC CANCER AND/OR PROGNOSIS OF PATIENTS WITH PANCREATIC CANCER BY BLOOD SAMPLES

(71) Applicants: Herlev Hospital, Herlev (DK); Ruprecht-Karls University of Heidelberg, Heidelberg (DE); Kræftens Bekæmpelse, Copenhagen Ø (DK); Rigshospitalet, Copenhagen Ø (DK)

(72) Inventors: Julia Sidenius Johansen, Frederiksberg (DK); Nicolai Aagaard Schultz, Frederiksberg C (DK); Jens Werner, Dossenheim (DE); Benny Vittrup Jensen, Roskilde (DK); Klaus K. Andersen, Frederiksberg (DK)

(21) Appl. No.: 14/372,350

(22) PCT Filed: Jan. 16, 2013
(86) PCT No.: PCT/DK2013/050014

§ 371 Date: Jul. 15, 2014

(30) Foreign Application Priority Data
Jan. 16, 2012 (DK) PA201270026
May 31, 2012 (DK) PA201270290

(51) Int. Cl. C12Q 1/68

(52) U.S. Cl. CPC C12Q 1/6886; C12Q 2600/178
USPC 506/9; 506/16; 435/287.2; 435/6.12

(57) ABSTRACT

The present invention relates to methods for improving the diagnosis and prognosis of patients with pancreatic carcinoma by making use of specific miRNA biomarkers associated with pancreatic carcinoma that may be identified based on a blood sample, in particular a whole blood, serum or plasma sample, from an individual.

All patent and non-patent references cited in the application are hereby incorporated by reference in their entirety.

### FIELD OF INVENTION

The present invention relates to a method for improving the diagnosis of and giving a prognosis for patients with pancreatic cancer. MicroRNA (miRNA) biomarkers and classifiers based on a specific miRNA expression pattern are disclosed herein, which distinguishes pancreatic cancer from normal pancreas and/or chronic pancreatitis when evaluated in samples from blood (whole blood, serum and plasma).

### BACKGROUND OF INVENTION

Worldwide over 277,000 patients developed pancreatic cancer (pancreatic carcinoma, PC) in 2008 (1) and PC is the 4^{th} most common cause of cancer death in United States (˜37.660) and Europe (˜75.000) (2,3). The prognosis is dismal (2,4). Surgery is the only potentially curative therapy because current chemotherapy has little effect (4,5). A small minority of patients with pancreatic cancer are actually cured, since most patients have locally advanced or metastatic pancreatic cancer at time of diagnosis and less than 20% can be operated with curative intent. Patients with pancreatic cancer have the poorest prognosis compared to patients with other types of adenocarcinoma; the median survival time is 6 months, and the 1- and 5-year survival is only 20% and 6%.

Tissues from pancreatic cancer contains an average of 63 genetic alterations and these define a core set of 12 cellular signaling pathways and processes that are each genetically altered in 67-100% of the patients (7).

MicroRNAs (miRNAs) are non-coding RNAs which regulate gene expression posttranscriptionally. MiRNAs play essential roles in basic biological functions such as cancer cell proliferation and differentiation, invasion, angiogenesis, and miRNAs also regulate epithelial-mesenchymal transition and cancer stem cells (8-13). 1527 human miRNAs sequences are described today (www.mirbase.org. Jan. 2, 2012).

It is often difficult to get useful biopsies of PC tissue from subjects suspected of having PC. A sensitive and specific diagnostic blood test for PC would therefore be very valuable. The use of miRNAs as biomarkers in blood samples is a new research field (14). Circulating miRNAs in plasma reflect miRNAs in tumor tissue, and miRNAs in blood are not degraded (14,15). Recent studies of patients with PC and healthy subjects have shown that high expression in plasma of miR-16, miR-18a, miR-20a, miR-21, miR-24, miR-25, miR-99a, miR-155, miR-181a, miR-181b, miR-185, miR-191, miR-196a and miR-210 and low expression of let-7 family and miR-146a had good sensitivity and specificity to identify PC from healthy subjects (16-22). Furthermore, the expressions of miR-21, let-7, and miR-196a were related to survival (20-22). MiRNAs from the inflammatory cells, like neutrophils and monocytes, which play important roles in cancer growth, progression and development of metastatic disease can be determined by analyzing whole blood instead of serum or plasma.

Novel strategies for early diagnosis of pancreatic cancer are urgently needed (4) in order to identify patients with pancreatic cancer at an early stage before the cancer has advanced locally or metastasized. Early diagnosis of pancreatic cancer is very difficult and there are no biomarkers in blood that can be used to identify patients with pancreatic cancer at an early stage (4,6). The aim of the present study was to identify new diagnostic and prognostic miRNAs in serum, plasma and whole blood from patients with pancreatic cancer.

### SUMMARY OF INVENTION

Efforts to make possible an early diagnosis of pancreas cancer are urgently needed, in order to improve the outcome of existing therapies.

Diagnosing pancreatic cancer via blood samples, blood being easily accessible and abundantly available, has the advantage of being a simple, fast, non-invasive and more economic method, when compared to the current state of the art which is to perform biopsies of the suspected pancreatic cancer.

The present inventors have further investigated in blood samples the miRNA expression profile in patients with pancreatic cancer (PC (comprising pancreatic adenocarcinoma, PAC and ampullary adenocarcinoma, AAC), in patients with chronic pancreatitis (CP) and in healthy subjects (HS) with normal pancreas (NP) (‘HS’ and ‘NP’ used interchangably) in order to identify specific miRNAs associated with each condition.

This has led to the identification of a deregulated subset of miRNAs associated with each of the above-mentioned conditions. These miRNAs are potentially useful in diagnosing a condition of the pancreas, such as pancreas cancer in samples of whole blood, plasma and/or serum.

Combined herewith efforts to make possible an improved prognosis for patients with pancreatic cancer have been made, in order to improve and specify the individual follow-up therapy after surgery and diagnosis and management after diagnosis of the condition for each patient with pancreatic cancer.

This has led to the identification of a subset of miRNAs associated with the survival of said PC patients as seen by the parameter “overall survival” (OS). These miRNAs are potentially useful in assessing or determining the prognosis of an individual patient with pancreatic cancer.

The present invention thus discloses a sensitive and specific means of separating patients with pancreatic cancer from healthy subjects with normal pancreas and/or from patients with chronic pancreatitis, by analysing a blood sample, which is simple, fast, non-invasive and economic. The inventors have found that a subset of specific miRNAs are differentially expressed in and associated with each of the above-mentioned conditions, efficiently separating the above-mentioned conditions of the pancreas by employing miRNA classifiers or biomarkers (alone or in ‘simple combinations’) capable of predicting which of the above categories or classes a certain sample obtained from an individual belongs to.

It is surprising that blood samples may be used as a means for diagnosing pancreatic cancer and for giving a prognosis of survival for an individual suffering here from. The state of the art comprises taking samples from affected pancreatic tissue and analysing this tissue sample in comparison to healthy pancreatic tissue, often from the same individual. A blood sample does not derive directly from the diseased tissue and blood is ubiquitous throughout the body, why it is surprising that blood, such as whole blood, serum or plasma may be used as sample material. Furthermore, the present invention comprises comparing the blood sample from the individual to be diagnosed or given a prognosis with a control sample. The control sample is miRNA from blood of either healthy individuals or a combined group of healthy individuals and individuals suffering from chronic pancreatitis.

By employing the prognostic miRNA biomarkers (alone or in ‘simple combinations’) disclosed herein, it is thus made possible to predict the prognosis of a diseased individual suffering from pancreatic cancer. The quality of said prediction is at least comparable to other prognostic biomarkers for PC, and in some embodiments yields an improved prognosis as compared to those provided thus far.

The present invention is in one aspect directed to the identification of prognostic miRNA biomarkers whose expression level is associated with estimating the prognosis of PC patients.

Accordingly, provided herein are methods for predicting the prognosis for a patient with pancreatic cancer, said method comprising measuring the expression level of at least one miRNA in a sample obtained from said individual, determining whether or not said sample is indicative of the individual having a certain predicted prognosis.

Said method may be a method for estimating the probability for a patient with pancreatic cancer of surviving for a certain time period.

Thus the present invention comprises a method for diagnosing if an individual has, or is at risk of developing, pancreatic cancer, and/or a method for giving a prognosis for the survival of the individual, said method comprising measuring the level of at least one miRNA in a blood sample obtained from said individual, wherein the at least one miRNA is selected from the group consisting of:
a) in a whole blood sample for diagnosis
   a. let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
b) in a serum sample for diagnosis
   a. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645; or
c) in a plasma sample for diagnosis miR-let7b, miR-10b*, miR-17, miR-19a, miR-19b, miR-20b, miR-24, miR-27a, miR-30d, miR-93, miR-106a, miR-126, miR-126*, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305; or
d) in a serum sample for prognosis of miR-19b, miR-27a, miR-30b, miR-30e-3p, miR-99b, miR-100, miR-181a, miR-185, miR-331-3p, miR-511, miR-362-3p, miR-758 and miR-1238; or
e) in a whole blood sample for prognosis miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636,
wherein the miRNA level, and/or the difference in the miRNA level, of at least one of said miRNAs compared to a control is indicative of said individual having, or being at risk of developing, pancreatic cancer and/or giving a prognosis for the survival of said individual.

The present invention is in one aspect directed to the development of a two-way miRNA classifier that distinguishes pancreatic cancer from normal pancreas and/or chronic pancreatitis, and comprises or consists of one or more miRNAs selected from the group consisting of the miRNAs cited above.

Further potential miRNA biomarkers deregulated in specific conditions of the pancreas are also disclosed herein, which are potentially useful for diagnosis of conditions of the pancreas and/or for the prognosis of individuals suffering here from.

The miRNA classifiers and/or biomarkers may be applied ex vivo to a sample obtained from an individual, in order to facilitate an early and accurate diagnosis of and/or prognosis for said individual. Said sample may be a blood sample from an individual, such as a whole blood sample or a serum or plasma sample, obtained from an individual.

Accordingly, provided herein are methods, devices and systems for diagnosing whether a subject has, or is at risk of developing, pancreatic cancer, comprising the steps of measuring the miRNA expression level in a sample obtained from an individual, and determining whether or not said sample is indicative of the individual of having, or being at risk of developing, pancreatic cancer.

Also provided are methods, devices and systems for predicting the prognosis for a patient with pancreatic cancer, comprising means for analysing the expression level of at least one miRNA in a sample obtained from an individual with pancreatic cancer, and means for determining the prognosis for said individual.

The diagnostic and prognostic methods may be used in combination with the CA 19.9 blood marker for pancreatic cancer.

The use of the herein disclosed miRNA classifiers and biomarkers can potentially drastically improve the diagnosis of pancreatic cancer and allow for an earlier diagnosis, and is as such useful as a stand-alone or an ‘add-on’ method to the existing diagnostic methods currently used for diagnosing pancreas cancer. Early diagnosis of a malignant condition of the pancreas is urgently needed in order to present pancreatic cancer patient to surgery at a less advanced stage.

The present invention is based on blood samples, having the advantage of providing a diagnostic tool which (a) is a simple, fast, non-invasive and economic; (b) may be used by a practitioner in a non-hospital setting (c) may be used in combination with other similar diagnostic tools providing a platform for screening and/or diagnosis multiple disease and/or disorders at the same time.

### DESCRIPTION OF DRAWINGS

FIG. 1. Discovery Study, serum. Box plots showing microRNAs expression in serum from patients with pancreatic cancer (n=139), patients with chronic pancreatitis (n=17) and healthy subjects (n=50). Comparison sorted by F-test, p-values. The TaqMan® Human MicroRNA assay was used and 3 different types of normalization was used (rank normalization, quantile normalization, and endogene controls) before statistical analysis.

FIG. 2. Discovery Study, whole blood. Box plots showing microRNAs expression in whole blood from patients with pancreatic cancer (n=41), patients with chronic pancreatitis (n=4) and healthy subjects (n=17). Comparison sorted by F-test, p-values. The TaqMan® Human MicroRNA assay was used and 3 different types of normalization was used (rank normalization, quantile normalization, and endogene controls) before statistical analysis.

FIG. 3: Boxplot of first 9 miRs from Table 6 (Example 2 & 4). The CT-values have been quantile normalized. The non-overlapping notches indicate significantly different medians. Above each panel the name of the miR is written together with the estimated effect (adjusted for the other significant miRs in the model for quantile normalized values) for an increase corresponding to the interquartile range. CP=Chronic pancreatitis, HS=healthy subject, JJ=internal control (persons initials).

FIG. 4: Pilot study samples for significant miRs using a) endogene controls, b) quantile normalization, c) rank normalization, d) 120 most expressed normalization, or e) raw values (whole-blood, cf. example 2).

FIG. 5: Whole blood diagnostic indexes I and II. Box-plots of bPANmiRC I and II using the samples from the “Discovery Study”, “Training Study” (=Discovery study+miRNAs from literature) and “Validation Study” (see example 4). The median score is the line in the middle of the box and the 25^{th} and 75^{th} percentile are the lower and upper part of the box. The whiskers are the 5^{th} and 95^{th} percentiles. Outliers are given as dots.

FIG. 6: ROC-curves for performance of bPANmiRC I and II indexes, and bPANmiRC I and II indexes in combination with CA 19-9 in the “Discovery Study”, “Training Study” and “Validation Study” (cf. example 4; whole blood).

FIG. 7: Serum diagnostic indexes. Box-plots of sPANmiRC I and III (cf. example 3 and table 13B) using the samples from the “Discovery Study”, either alone or combined with serum CA 19.9 according to PC (all stages combined) or controls (CP and HS combined). The median score is the line in the middle of the box and the 25^{th} and 75^{th} percentile are the lower and upper part of the box. The whiskers are the 5^{th} and 95^{th} percentiles. Outliers are given as dots.

FIG. 8: Serum diagnostic indexes. Box-plots of sPANmiRC I and III (cf. example 3 and table 13B) using the samples from the “Training Study”, either alone or combined with serum CA 19.9 according to PC (all stages combined) or controls (CP and HS combined). The median score is the line in the middle of the box and the 25^{th} and 75^{th} percentile are the lower and upper part of the box. The whiskers are the 5^{th} and 95^{th} percentiles. Outliers are given as dots.

FIG. 9: Serum diagnostic indexes. ROC-curves for performance of the Diagnostic indexes in the “Discovery Study—Fluidigm method” and “Training Study” (cf. table 13B) alone or in combination with serum CA 19.9.

### DEFINITIONS

Statistical classification is a procedure in which individual items are placed into groups based on quantitative information on one or more characteristics inherent in the items (referred to as traits, variables, characters, etc) and based on a training set of previously labeled items.

A classifier is a prediction model which may distinguish between or characterize samples by classifying a given sample into a predetermined class based on certain characteristics of said sample. A two-way classifier classifies a given sample into one of two predetermined classes, and a three-way classifier classifies a given sample into one of three predetermined classes.

The terms distinction, differentiation, separation, classification and characterisation of a sample are used herein as being capable of predicting with a relatively high sensitivity and specificity if a given sample of unknown diagnosis belongs to the class of pancreatic cancer, chronic pancreatitis and/or normal pancreas. The output may be given as a probability of belonging to either class of between 0-1 (for classifiers), or may be estimated directly based on differences in expression levels (for biomarkers).

A ‘biomarker’ may be defined as a biological molecule found in blood, other body fluids, or tissues that is an indicator of a normal or abnormal process, or of a condition or disease. A biomarker may be used to foresee how well the body responds to a treatment for a disease or condition, or may be used to associate a certain disease or condition to a certain value of said biomarker found in e.g. a tissue sample. Biomarkers are also called molecular markers and signature molecules.

A ‘blood sample’ is a sample of blood taken from an individual. The sample may comprise arterial, capillary and/or venous blood. The sample may be used as a whole blood sample, or may be separated to yield plasma and/or serum. To do so blood is centrifuged to remove cellular components. Anti-coagulated blood yields plasma containing fibrinogen and clotting factors. Coagulated blood (clotted blood) yields serum without fibrinogen, although some clotting factors remain. Blood, once drawn, may be mixed with e.g. EDTA or Lithium Heparin to prevent clotting, or other factors to prevent the degradation of RNA and specifically miRNA in the samples. Pre-prepared sampling devices may be used for storage of the samples, e.g. pre-prepared tubes with EDTA or PAXgene Blood RNA tubes (Qiagen) for stabilization of RNA. The blood, serum and/or plasma sample may be fresh, frozen or fixed.

‘Collection media’ as used herein denotes any solution suitable for collecting, storing or extracting of a sample for immediate or later retrieval of RNA from said sample.

‘Deregulated’ means that the expression of a gene or a gene product is altered from its normal baseline levels; comprising both up- and down-regulated.

The term “Individual” refers to vertebrates, particular members of the mammalian species, preferably primates including humans. As used herein, ‘subject’, ‘individual’ and “patient” may be used interchangeably.

The term “Kit of parts” as used herein provides a device for measuring the expression level of at least one miRNA as identified herein, and at least one additional component.

The additional component may be used simultaneously, sequentially or separately with the device. The additional component may in one embodiment be means for extracting RNA, such as miRNA, from a sample; reagents for performing microarray analysis, array analysis, reagents for performing quantitative real time polymerase chain reaction (QPCR) analysis and/or instructions for use of the device and/or additional components.

A ‘probe’ as used herein refers to a hybridization probe. A hybridization probe is a (single-stranded) fragment of DNA or RNA of variable length (usually 100-1000 bases long), which is used in DNA or RNA samples to detect the presence of nucleotide sequences (the DNA target) that are complementary to the sequence in the probe. The probe thereby hybridizes to single-stranded nucleic acid (DNA or RNA) whose base sequence allows probe-target base pairing due to complementarity between the probe and target. To detect hybridization of the probe to its target sequence, the probe is tagged (or labelled) with a molecular marker of either radioactive or fluorescent molecules. DNA sequences or RNA transcripts that have moderate to high sequence similarity to the probe are then detected by visualizing the hybridized probe. Hybridization probes used in DNA microarrays refer to DNA covalently attached to an inert surface, such as coated glass slides or gene chips, and to which a mobile cDNA target is hybridized.

Due to the imprecision of standard analytical methods, molecular weights and lengths of polymers are understood to be approximate values. When such a value is expressed as “about” X or “approximately” X, the stated value of X will be understood to be accurate to +/−20%, such as +/−10%, for example +/−5%.

### DETAILED DESCRIPTION OF THE INVENTION

### The Pancreas

The pancreas is a gland organ in the digestive and endocrine system of vertebrates. It is both an endocrine gland producing several important hormones, including insulin, glucagon, and somatostatin, as well as an exocrine gland, secreting pancreatic juice containing digestive enzymes that pass to the small intestine. These enzymes help to further break down the carbohydrates, proteins, and fats in the chyme.

Microscopically, stained sections of the pancreas reveal two different types of parenchymal tissue. Lightly staining clusters of cells are called islets of Langerhans, which produce hormones that underlie the endocrine functions of the pancreas. Darker staining cells form acini connected to ducts. Acinar cells belong to the exocrine pancreas and secrete digestive enzymes into the gut via a system of ducts.

Four main cell types exist in the islets of Langerhans that can be classified by their secretion: α (alpha) cells secrete glucagon (increase glucose in blood), β (beta) cells secrete insulin (decrease glucose in blood), δ (delta) cells secrete somatostatin (regulates α and β cells), and PP cells secrete pancreatic polypeptide.

The pancreas receives regulatory innervation via hormones in the blood and through the autonomic nervous system. These two inputs regulate the secretory activity of the pancreas. The pancreas lies in the epigastrium and left hypochondrium areas of the abdomen. The head lies within the concavity of the duodenum. The uncinate process emerges from the lower part of head, and lies deep to superior mesenteric vessels. The neck is the constricted part between the head and the body. The body lies behind the stomach. The tail is the left end of the pancreas. It lies in contact with the spleen and runs in the lienorenal ligament.

### Pancreatic Cancer

Neoplasia or cancer is the abnormal proliferation of cells, resulting in a structure known as a neoplasm. The growth of this clone of cells exceeds, and is uncoordinated with, that of the normal tissues around it. It usually causes a lump or tumour. Neoplasias may be benign (adenoma) or malignant (carcinoma).

Pancreatic or pancreas neoplasia, pancreatic or pancreas cancer (PC), pancreatic or pancreas carcinoma may be used interchangeably throughout the present application. Normal pancreas is abbreviated NP, and is found in healthy Subjects (HS)—the terms NP and HS are used interchangeably herein.

Pancreatic cancer is a malignant neoplasm of the pancreas. Patients diagnosed with pancreatic cancer have a poor prognosis, partly because the cancer usually causes no specific symptoms early on, leading to locally advanced or metastatic disease at the time of diagnosis. Median survival from diagnosis of pancreatic cancer is around 3 to 6 months; 5-year survival is less than 5%. Pancreatic cancer has one of the highest fatality rates of all cancers, and is the fourth-highest cancer killer in the US and Europe.

The vast majority; about 95% of exocrine pancreatic cancers are pancreatic adenocarcinomas; PAC (also known as pancreatic ductal adenocarcinoma, PDAC). Accordingly, PC and PAC are often used as synonyms. The remaining 5% include adenosquamous carcinomas, signet ring cell carcinomas, hepatoid carcinomas, colloid carcinomas, undifferentiated carcinomas, and undifferentiated carcinomas with osteoclast-like giant cells. Exocrine pancreatic tumors are far more common than pancreatic endocrine tumors, which make up about 1% of total cases.

Desmoplasia is the growth of fibrous or connective tissue. It is also called desmoplastic reaction to emphasize that it is secondary to a neoplasm, causing dense fibrosis around the tumor. Desmoplasia is usually only associated with malignant neoplasms, such as pancreatic cancer which can evoke a fibrosis response by invading healthy tissue.

Treatment of pancreatic cancer depends on the stage of the cancer. The Whipple procedure is the most common surgical treatment for cancers involving the head of the pancreas. This procedure involves removing the pancreatic head and the curve of the duodenum together (pancreato-duodenectomy), making a bypass for food from stomach to jejunum (gastro-jejunostomy) and attaching a loop of jejunum to the cystic duct to drain bile (cholecysto-jejunostomy). It can be performed only if the patient is likely to survive major surgery and if the cancer is localized without invading local structures or metastasizing. It can, therefore, be performed in only the minority of cases. Cancers of the tail of the pancreas can be resected using a procedure known as a distal pancreatectomy. Recently, localized cancers of the pancreas have been resected using minimally invasive (laparoscopic) approaches. Surgery can be performed for palliation, if the malignancy is invading or compressing the duodenum or colon. In that case, bypass surgery might overcome the obstruction and improve quality of life, but it is not intended as a cure.

After surgery, adjuvant chemotherapy has been shown to significantly increase the 5-year survival, and should be offered if the patient is fit after surgery. Addition of radiation therapy is a hotly debated topic, due to the lack of any large randomized studies to show any survival benefit of this strategy. In patients not suitable for resection with curative intent, palliative chemotherapy may be used to improve quality of life and gain a modest survival benefit.

### Ampullary Adenocarcinoma

Ampullary adenocarcinomas (A-AC or AAC); also known as adenocarcinoma of the Ampulla of Vater, is a malignant tumour arising in the last centimeter of the common bile duct, where it passes through the wall of the duodenum and ampullary papilla. The pancreatic duct (of Wirsung) and common bile duct merge and exit by way of the ampulla into the duodenum. The ductal epithelium in these areas is columnar and resembles that of the lower common bile duct.

AAC is relatively uncommon, accounting for approximately 0.2% of gastrointestinal tract malignancies and approximately 7% of all periampullary carcinomas.

The prognosis of AAC is better than for PAC with a 5-years survival after surgery of 40%. One of the reasons is that even small AAC cause jaundice so more patients are operated at an early tumour stage and without lymph node metastasis.

### Pancreatitis

Chronic pancreatitis (CP) is commonly defined as a continuing, chronic inflammatory process of the pancreas, characterized by irreversible morphological changes. This chronic inflammation can lead to chronic abdominal pain and/or impairment of endocrine and exocrine function of the pancreas. Chronic pancreatitis usually is envisioned as an atrophic fibrotic gland with dilated ducts and calcifications. However, findings on conventional diagnostic studies may be normal in the early stages of chronic pancreatitis, as the inflammatory changes can be seen only by histologic examination.

By definition, chronic pancreatitis is a completely different process from acute pancreatitis. In acute pancreatitis, the patient presents with acute and severe abdominal pain, nausea, and vomiting. The pancreas is acutely inflamed (neutrophils and oedema), and the serum levels of pancreatic enzymes (amylase and lipase) are elevated. Full recovery is observed in most patients with acute pancreatitis, whereas in chronic pancreatitis, the primary process is a chronic, irreversible inflammation (monocyte and lymphocyte) that leads to fibrosis with calcification. The patient with chronic pancreatitis clinically presents with chronic abdominal pain and normal or mildly elevated pancreatic enzyme levels; when the pancreas loses its endocrine and exocrine function, the patient presents with diabetes mellitus and steatorrhea.

### Diagnosing Pancreatic Cancer at Present

Pancreatic cancer is sometimes called a “silent killer” because early pancreatic cancer often does not cause symptoms, and the later symptoms are usually nonspecific and varied. Therefore, pancreatic cancer is often not diagnosed until it is advanced. The clinical and histological similarity between pancreatic cancer and chronic pancreatitis adds another dimension to the diagnostic challenge.

Common symptoms of PC include:
Pain in the upper abdomen that typically radiates to the back (seen in carcinoma of the body or tail of the pancreas).
Loss of appetite and/or nausea and vomiting.
Significant weight loss.
Painless jaundice (yellow skin/eyes, dark urine) when a cancer of the head of the pancreas (about 60% of cases) obstructs the common bile duct as it runs through the pancreas. This may also cause pale-colored stool and steatorrhea.
Trousseau sign, in which blood clots form spontaneously in the portal blood vessels, the deep veins of the extremities, or the superficial veins anywhere on the body, is sometimes associated with pancreatic cancer.
Diabetes mellitus, or elevated blood sugar levels. Many patients with pancreatic cancer develop diabetes months to even years before they are diagnosed with pancreatic cancer, suggesting new onset diabetes in an elderly individual may be an early warning sign of pancreatic cancer.

The initial presentation varies according to location of the cancer. Malignancies in the pancreatic body or tail usually present with pain and weight loss, while those in the head of the gland typically present with steatorrhea, weight loss, and jaundice. The recent onset of atypical diabetes mellitus, a history of recent but unexplained thrombophlebitis (Trousseau sign), or a previous attack of pancreatitis are sometimes noted. Courvoisier sign defines the presence of jaundice and a painlessly distended gallbladder as strongly indicative of pancreatic cancer, and may be used to distinguish pancreatic cancer from gallstones. Tiredness, irritability and difficulty eating because of pain also exist. Pancreatic cancer is often discovered during the course of the evaluation of aforementioned symptoms.

Liver function tests can show a combination of results indicative of bile duct obstruction (raised conjugated bilirubin, y-glutamyl transpeptidase and alkaline phosphatase levels).

Imaging studies, such as computed tomography (CT scan) and endoscopic ultrasound (EUS) can be used to identify the location and form of the cancer.

An assessment of risk factors may also help make a diagnosis, comprising the occurrence of pancreatic cancer in the family, age above 60 years, male gender, smoking, obesity, diabetes mellitus, chronic pancreatitis, Helicobacter pylori infection, gingivitis or periodontal disease, diets low in vegetables and fruits, high in red meat, and/or high in sugar-sweetened drinks.

A definitive diagnosis is made by an endoscopic needle biopsy or surgical excision of the radiologically suspicious tissue. Endoscopic ultrasound is often used to visually guide the needle biopsy procedure.

The most common form of pancreatic cancer (ductal adenocarcinoma) is typically characterized by moderately to poorly differentiated glandular structures on microscopic examination. Pancreatic cancer has an immunohistochemical profile that is similar to hepatobiliary cancers (e.g. cholangiocarcinoma) and some stomach cancers; thus, it may not always be possible to be certain that a tumour found in the pancreas arose from it.

CA 19-9 (carbohydrate antigen 19.9) is a tumor marker or biomarker that is frequently elevated in pancreatic cancer (detectable in the serum). It is used mainly for monitoring and early detection of recurrence after treatment of patients with known PC. However, it lacks sensitivity and specificity. CA 19-9 might be normal early in the course, and could also be elevated because of benign causes of biliary obstruction. Further 10% of patients with PC are unable to produce CA 19-9.

Thus, novel strategies for early diagnosis of patients with pancreatic cancer are urgently needed. The use of miRNA expression levels as biomarkers in blood samples is an emerging research field aimed at improving the diagnostic tools for pancreas cancer.

The methods disclosed herein provide a tool for improving the early diagnosis of pancreatic cancer, thus improving prognosis of affected individuals. The miRNA classifiers and/or biomarkers as disclosed herein may in one embodiment be used in the clinic alone (standalone diagnostic); i.e. without employing further diagnostic methods.

In another embodiment, the miRNA classifiers and/or biomarkers as disclosed herein may be used in the clinic as an add-on or supplementary diagnostic tool or method, which improves the diagnosis of pancreas cancer by combining the output of said miRNA classifier and/or biomarker level with the output of one or more of the above-mentioned conventional diagnostic techniques to improve the accuracy of said diagnosis of pancreas cancer. In an embodiment the methods, miRNA classifiers and/or biomarkers as disclosed herein may be used in combination with the CA 19-9 tumor marker in blood, such as serum, plasma or whole blood.

### MicroRNA

MicroRNAs (miRNA) are single-stranded RNA molecules of about 19-25 nucleotides in length, which regulate gene expression. miRNAs are either expressed from non-protein-coding transcripts or mostly expressed from protein coding transcripts. They are processed from primary transcripts known as pri-miRNA to shorter stem-loop structures called pre-miRNA and finally to functional mature miRNA. Mature miRNA molecules are partially complementary to one or more messenger RNA (mRNA) molecules, and their main function is to inhibit gene expression. This may occur by preventing mRNA translation or increasing mRNA turnover/degradation.

The transcripts encoding miRNAs are much longer than the processed mature miRNA molecule; miRNAs are first transcribed as primary transcripts or pri-miRNA with a cap and poly-A tail by RNA polymerase II and processed to short, 70-nucleotide stem-loop structures known as pre-miRNA in the cell nucleus. This processing is performed in animals (including humans) by a protein complex known as the Microprocessor complex, consisting of the ribonuclease III Drosha and the double-stranded RNA binding protein Pasha. These pre-miRNAs are then exported to the cytoplasm by Exportin-5/Ran-GTP and processed to mature miRNAs by interaction with the ribonuclease III Dicer and separation of the miRNA duplexes. The mature single-stranded miRNA is incorporated into a RNA-induced silencing complex (RISC)-like ribonucleoprotein particle (miRNP). The RISC complex is responsible for the gene silencing observed due to miRNA expression and RNA interference. The pathway is different for miRNAs derived from intronic stem-loops; these are processed by Dicer but not by Drosha.

When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end. The remaining strand, known as the anti-guide or passenger strand, is degraded as a RISC complex substrate. After integration into the active RISC complex, miRNAs base pair with their complementary mRNA molecules. This may induce mRNA degradation by argonaute proteins, the catalytically active members of the RISC complex, or it may inhibit mRNA translation into proteins without mRNA degradation.

The function of miRNAs appears to be mainly in gene regulation. For that purpose, a miRNA is (partly) complementary to a part of one or more mRNAs. Animal (including human) miRNAs are usually complementary to a site in the 3′ UTR. The annealing of the miRNA to the mRNA then inhibits protein translation, and sometimes facilitates cleavage of the mRNA (depending on the degree of complementarity). In such cases, the formation of the double-stranded RNA through the binding of the miRNA to mRNA inhibits the mRNA transcript through a process similar to RNA interference (RNAi). Further, miRNAs may regulate gene expression post-transcriptionally at the level of translational inhibition at P-bodies. These are regions within the cytoplasm consisting of many enzymes involved in mRNA turnover; P bodies are likely the site of miRNA action, as miRNA-targeted mRNAs are recruited to P bodies and degraded or sequestered from the translational machinery. In other cases it is believed that the miRNA complex blocks the protein translation machinery or otherwise prevents protein translation without causing the mRNA to be degraded. miRNAs may also target methylation of genomic sites which correspond to targeted mRNAs. miRNAs function in association with a complement of proteins collectively termed the miRNP (miRNA ribonucleoprotein complex).

Under a standard nomenclature system, miRNA names are assigned to experimentally confirmed miRNAs before publication of their discovery. The prefix “mir” is followed by a dash and a number, the latter often indicating order of naming. For example, mir-123 was named and likely discovered prior to mir-456. The uncapitalized “mir-” refers to the pre-miRNA, while a capitalized “miR-” refers to the mature form. miRNAs with nearly identical sequences bar one or two nucleotides are annotated with an additional lower case letter. For example, miR-123a would be closely related to miR-123b. miRNAs that are 100% identical but are encoded at different places in the genome are indicated with additional dash-number suffix: miR-123-1 and miR-123-2 are identical but are produced from different pre-miRNAs. Species of origin is designated with a three-letter prefix, e.g., hsa-miR-123 would be from human (Homo sapiens) and oar-miR-123 would be a sheep (Ovis aries) miRNA. Other common prefixes include ‘v’ for viral (miRNA encoded by a viral genome) and ‘d’ for Drosophila miRNA. microRNAs originating from the 3′ or 5′ end of a pre-miRNA are denoted with a -3p or -5p suffix. (In the past, this distinction was also made with ‘s’ (sense) and ‘as’ (antisense)). An asterisk following the name indicates that the miRNA is an anti-miRNA to the miRNA without an asterisk (e.g. miR-123* is an anti-miRNA to miR-123). When relative expression levels are known, an asterisk following the name indicates a miRNA expressed at low levels relative to the miRNA in the opposite arm of a hairpin. For example, miR-123 and miR-123* would share a pre-miRNA hairpin, but relatively more miR-123 would be found in the cell.

As used herein, it is understood that ‘miR-’ and ‘hsa-miR’ is used interchangeably; the results of the present invention are obtained from human samples and human miRNAs are examined.

Also, it is understood that e.g. hsa-miR-123 is identical to miR-123, and that this may also be denoted miR.123 as well as miR-123 or hsa-miR-123 or hsa.miR.123.

miRBase is the central online repository for microRNA (miRNA) nomenclature, sequence data, annotation and target prediction, and may be accessed via http://www.mirbase.org/. The miRNA names used herein throughout can be accessed via this link, and specifics retrieved. See also Griffiths-Jones et al, “miRBase: tools for microRNA genomics”, Nucleic Acids Research, 2008, Vol. 36, Database issue D154-D158.

### Biomarker

A biomarker, or biological marker, is in general a substance used as an indicator of a biological state. It is a characteristic that is objectively measured and evaluated as an indicator of normal biological processes, pathogenic processes, or pharmacologic responses to a therapeutic intervention.

More specifically, a biomarker indicates a change in expression or state of a protein or miRNA that correlates with the risk or progression of a disease, or with the susceptibility of the disease to a given treatment.

A biomarker, such as a miRNA biomarker, may be correlated to a certain condition based on differences in miRNA expression levels between a sample and a control. If a certain miRNA biomarker is found to be deregulated in a sample as compared to a (normal) control level, the sample has a certain probability of being associated with a certain condition.

According to the present invention, the miRNA biomarkers identified herein in blood are able to correlate a deregulated expression level of said miRNA to a diagnosis of pancreatic cancer (such as PAC and/or AAC).

It follows that the expression of one biomarker may in itself be deregulated in a condition (e.g. cancer) as compared to another condition (e.g. control); or it may be the relationship between the expression levels of two or more biomarkers that is telling of a particular condition; i.e. the relative difference in expression levels between two biomarkers.

Prognostic and Diagnostic miRNA Biomarkers of the Present Invention

The present invention is directed to identification of miRNA biomarkers with the potential to distinguish patients with pancreatic carcinoma (pancreatic and/or ampullary adenocarcinoma) from subjects with normal pancreas and/or chronic pancreatitis in order to make an early and sensitive diagnosis; and to give a prognosis for the overall survival of an individual having been diagnosed with pancreatic cancer. The miRNAs of the present invention can be identified based on a blood sample, wherein said blood sample may be whole blood, serum or plasma.

The present invention is thus in one aspect directed to miRNA biomarkers that may be used to:
a) distinguish in a serum sample between patients with pancreatic carcinoma (PAC and/or AAC) and subjects with normal pancreas and/or chronic pancreatitis, and comprises or consists of
   a. miR-25, miR-26a, miR-26b, miR-27a, miR-29c, miR-30a-5p, miR-106a, miR-195, miR-212, miR-320, miR-323-3p, miR-345, miR-483-5p, miR-485-3p, miR-590-5p, miR-618, miR-638, and miR-645; and/or
   b. let-7b, miR-24, miR-25, miR-26a, miR-26b, miR-27a, miR-29c, miR-30a-5p, miR-30e.3p, miR-106a, miR-148a, miR-185, miR-191, miR-195, miR-212, miR-320, miR-323-3p, miR-345, miR-483-5p, miR-485-3p, miR-590-5p, miR-618, miR-638, and miR-645; and/or
   c. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645; and/or
   d. miR-16, miR-27a, miR-30a.5p, miR-20a, miR-25 and miR-483.5p; and/or
   e. miR-16, miR-27a, miR-30a.5p, miR-323.3p, miR-20a, miR-29c and miR-483.5p; and/or
   f. miR-16, miR-24, miR-27a, miR-30a.5p, miR-485.3p, miR-20a, miR-25, miR-29c, miR-99a, miR-345, miR-483.5p and miR-618; and/or
   g. miR-16, miR-24, miR-27a, miR-30a.5p, miR-323.3p, miR-20a, miR-25, miR-29c and miR-483.5p; and/or
   h. miR-24, miR-27a, miR-323.3p, miR-20a and miR-483.5p; and/or
   i. miR-16, miR-27a, miR-25, miR-29c and miR-483.5p; and/or
   j. miR-16, miR-18a, miR-24, miR-27a, miR-30a.5p, miR-323.3p, miR-20a, miR-25, miR-29c, miR-30e.3p, miR-99a, miR-345 and miR-483.5p; and/or
   k. miR-16, miR-18a, miR-24, miR-27a, miR-30a.5p, miR-323.3p, miR-20a, miR-25, miR-29c, miR-191, miR-345 and miR-483.5p; or
   l. let-7b, miR-16, miR-18a, miR-20a, miR-24, miR-25, miR-26a, miR-26b, miR-27a, miR-29c, miR-30a-5p, miR-30e.3p, miR-99a, miR-106a, miR-181a, miR-185, miR-191, miR-195, miR-323-3p, miR-345, miR-483-5p, miR-485-3p, miR-590-5p and miR-618; or
   m. miR-16, miR-18a, miR-24, miR-26a, miR-26b, miR-27a, miR-30a.5p, miR-30e.3p, miR-99a, miR-106a, miR-323.3p, miR-20a, miR-25, miR-29c, miR-181a, miR-185, miR-191, miR-195, miR-345, miR-483-5p, miR-485-3p and miR-590.5p; or
   n. miR-16, miR-18.a, miR-20a, miR-24, miR-25, miR-27a, miR-29c, miR-30a.5p, miR-191, miR-323.3p, miR-345, and miR-483.5p.
   o. miR-16, miR-20a, miR-25, miR-27a, miR-30a.5p, miR-99a, miR-195, miR-483.5p and miR-618
   p. miR-16, miR-20a, miR-24, miR-25, miR-30a.5p, miR-30e.3p, miR-106a, miR-195, miR-345, and miR-483.5p
   q. miR-16, miR-20a, miR-24, miR-25, miR-27a, miR-29c, miR-30a.5p, miR-30e.3p, miR-185, miR-195, miR-323.3p, miR-345, miR-483.5p
b) distinguish in a whole blood sample between patients with pancreatic carcinoma (PAC and/or AAC) and subjects with normal pancreas and/or chronic pancreatitis, and comprises or consists of
   a. miR-20a, miR-30c, miR-31, miR-150, miR-190, and miR-196b; and/or
   b. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
   c. let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-505, miR-508.3p, miR-539, miR-582.3p, miR-625, miR-628.3p, miR-636, miR-769.5p, miR-885.5p, miR-935 and miR-941; and/or
   d. let-7g, miR-26b, miR-30b, miR-31, miR-34a, miR-122, miR-126*, miR-145, miR-150, miR-223, miR-505, miR-636 and miR-885.5p; and/or
   e. miR-122, miR-34a, miR-145, miR-636, miR-223, miR-26b, miR-885.5p, miR-150, miR-126* and miR-505; and/or
   f. miR-150, miR-30b, miR-145 and miR-223; and/or
   g. miR-150, miR-636, miR-145 and miR-223; and/or
   h. miR-935, miR-885.5p, miR-769.5p, miR-34a, miR-145, miR-31*, miR-31, miR-199b.5p and miR-150; and/or
   i. miR-935, miR-885.5p, miR-769.5p, miR-34a, miR-145, miR-31*, miR-31, miR-199b.5p, miR-150, miR-93, miR-636, miR-582.3p, miR-126* and miR-122; and/or
   j. let-7g, miR-636, miR-769.5p, miR-93, miR-122, miR-508.3p, miR-199b.5p, miR-935, miR-885.5p, miR-34a, miR-223, miR-144*, miR-145, miR-126*; or
c) distinguish in a plasma sample between patients with pancreatic carcinoma (PAC and/or AAC) and normal pancreas and/or chronic pancreatitis, and comprises or consists of miR-let7b, miR-10b*, miR-17, miR-19a, miR-19b, miR-20b, miR-24, miR-27a, miR-30d, miR-93, miR-106a, miR-126, miR-126*, miR-1395p, miR-140-3p, miR-140.5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305; or
d) give a prognosis in a serum sample for the overall survival of the sampled individual with pancreatic cancer, and comprises or consists of miR-19b, miR-27a, miR-30b, miR-30e-3p, miR-99b, miR-100, miR-181a, miR-185, miR-331-3p, miR-511, miR-362-3p, miR-758 and miR-1238; and/or
e) give a prognosis in a whole blood sample for the overall survival of the sampled individual with pancreatic cancer, and comprises or consists of miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636.

It is contemplated that the expression level of at least one of said miRNAs in one embodiment is measured in a blood sample from an individual, and said miRNA expression level as compared to a control or baseline level is then associated with a specific condition. Said condition may in one embodiment be pancreatic carcinoma, ampullary adenocarcinoma, pancreatic cancer and/or chronic pancreatitis.

In a particular embodiment, the difference between the expression levels of two miRNAs is calculated; wherein said difference in expression levels between said two miRNAs may be used to correlate said difference in miRNA expression level to a certain condition of the pancreas. Said difference may thus be a relative difference.

In one embodiment, said biomarkers are used in combination (‘simple combination’); i.e. the expression level of at least the two miRNAs according to a) to h) immediately herein above are both used in combination to distinguish or separate the potential conditions of the pancreas.

In another embodiment any of the above embodiments a) to e) are used in combination with each other, i.e. the biomarkers for diagnosing pancreatic cancer that may or may not be specific for e.g. serum (singly or in the combinations stated above) are used in combination with the biomarkers listed for whole blood and/or plasma. Also it is contemplated that e.g. the biomarkers for diagnosing are used in combination with the biomarkers for giving prognosis e.g. the biomarkers found in serum that are specific for giving a diagnosis are combined with the biomarkers found in serum that are specific in relation to giving a prognosis. Thus it is contemplated to combine at least the following embodiments in relation to diagnosing pancreatic cancer: a) with b); a) with c); a) with b) and c); or b) with c). Likewise it is contemplated to combine for giving a prognosis for an individual having pancreatic cancer a prognosis for survival by combining d) and e). In a further embodiment it is contemplated to give both a prognosis and diagnosis by combining at least two of either a), b), or c) with either d) or e). A further embodiment includes any of these combinations may be further combined with additional biomarkers such as CA 19-9 concentrations in whole blood, serum or plasma.

In a further embodiment the expression level of at least one or more of the following miRNAs is used as a biomarker in a serum sample from an individual to distinguish between PC and the combined group of NP and CP: i) miR-212, miR-19a, miR-30a-5p, miR-378, miR-320, miR-483-5p, and miR-let-7b, or ii) miR-let7b, miR-19a, miR-30a.5p, miR-212, miR-320, miR-378, and miR-483.5p, or iii) miR-let7b, miR-16, miR-20a, miR-21, miR-25, miR-27a, miR-30e.3p, miR-106b, miR-146a, miR-195, miR-212, miR-320, miR-338.5p, miR-378, miR-483.5p, miR-485, miR-638, miR-645, or iv) miR-25, miR-26a, miR-26b, miR-29c, miR-30a.5p, miR-106a, miR-195, miR-212, miR-320, miR-323.3p, miR-345, miR-483.5p, miR-618, miR-638, and miR-645, or v) miR-25, miR-27a, miR-106a, miR-195, miR-212, miR-323.3p, miR-485, and miR-590.5p, or vi) miR-195, miR-212, miR-483.5p, and miR-645.

In a further embodiment the expression level of at least one or more of the following miRNAs is used as a biomarker in a serum sample from an individual for prognosis of overall survival: i) miR-let7g, miR-16, miR-20a, miR-21, miR-30e.3p, miR-100, miR-146a, miR-146b.5p, miR-148a, miR-181a, miR-320, miR-328, miR-362.3p, and miR-511, or ii) miR-27a, miR-100, miR-181a, miR-362.3p, miR-511, miR-758, and miR-1238, or iii) miR-30e.3p and miR-99b.

In a further embodiment the expression level of at least the following miRNA is used as a biomarker in a serum sample from an individual for prognosis of overall survival: miR-19b.

The expression of any given biomarker of the present invention may in one embodiment be either increased or decreased in a sample from a patient with pancreatic cancer as compared to a control sample. Thus, the expression level of any given biomarker may be associated pancreatic cancer and used for diagnosing the same.

In a particular embodiment, the decreased expression level of at least one miRNA of the group consisting of miR-20a, miR-31, miR-150, miR-190, mir-196b, let-7b, let-7g, miR-9*, miR-19b, miR-23a, miR-24.2*, miR-31, miR-31*, miR-93, miR-143, miR-144*, miR-342.5p, miR-345, miR-362.3p, miR-374b*, miR-508.3p, miR-539, miR-628.3p, miR-636 and miR-935 is associated with a diagnosis of pancreatic cancer. Said expression may be measured on a whole blood sample.

In another particular embodiment, the increased expression level of at least one miRNA of the group consisting of miR-30c, miR-26b, miR-30b, miR-34a, miR-122, miR-126*, miR-128, miR-145, miR-186, miR-199b.5p, miR-223, miR-223*, miR-505, miR-582.3p, miR-625, miR-636, miR-769.5p, miR-885.5p and miR-941 is associated with a diagnosis of pancreatic cancer. Said expression may be measured on a whole blood sample.

It is contemplated that the expression level of at least one of said miRNAs in one embodiment is measured in a sample from an individual with pancreatic cancer, and said miRNA expression level is then associated with a prognosis.

Said prognosis may be defined as the predicted overall survival (OS) and/or survival at 2 years follow-up.

Said prognosis may be a graduation between ‘poor’ and ‘good’, it may be expressed in months or years expected survival, or it may be defined as a probability of surviving a certain time period expressed in months or years.

In one embodiment, the prognosis as defined herein is expressed as a probability of surviving a certain time period expressed in months or years. Said time period may be defined as 1½-months survival probability, 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability. Said probability of survival after a certain time period may be in the range of 0.01 to 0.1, such as 0.1 to 0.2, for example 0.2 to 0.3, such as 0.3 to 0.4, for example 0.4 to 0.5, such as 0.5 to 0.6, for example 0.6 to 0.7, such as 0.7 to 0.8, for example 0.8 to 0.85, such as 0.85 to 0.9, for example 0.9 to 0.91, such as 0.91 to 0.92, for example 0.92 to 0.93, such as 0.93 to 0.94, for example 0.94 to 0.95, such as 0.95 to 0.96, for example 0.96 to 0.97, such as 0.97 to 0.98, for example 0.98 to 0.99, such as 0.99 to 1.0.

Said time period may be calculated starting from time of diagnosis, time of surgery or time of analysis/evaluation.

The 3-months survival probability may in one embodiment be between 0.9 and 1.0. The 1-year survival probability may in one embodiment be between 0.2 and 0.9. The 10-year survival probability may in one embodiment be between 0.01 and 0.6.

It follows that a probability is expressed in a value of between 0-100, where 100 is a high probability of survival the indicated time period (good prognosis), and 0 is a low probability (poor prognosis).

The miRNA biomarkers as disclosed herein may in one embodiment be used (or measured; correlated) alone.

In one embodiment, said biomarkers are used in combination (′simple combination′), comprising at least two miRNA biomarkers. It follows that the expression level of two or more of the miRNAs according to the present invention is measured and correlated to the expected survival or prognosis.

### Classifier

Classifiers are relationships between sets of input variables, usually known as features, and discrete output variables, known as classes. Classes are often centered on the key questions of who, what, where and when. A classifier can intuitively be thought of as offering an opinion about whether, for instance, an individual associated with a given feature set is a member of a given class.

In other words, a classifier is a predictive model that attempts to describe one column (the label) in terms of others (the attributes). A classifier is constructed from data where the label is known, and may be later applied to predict label values for new data where the label is unknown. Internally, a classifier is an algorithm or mathematical formula that predicts one discrete value for each input row. Classifiers may also produce probability estimates for each value of the label.

### Sensitivity and Specificity

Sensitivity and specificity are statistical measures of the performance of a binary classification test. The sensitivity (also called recall rate in some fields) measures the proportion of actual positives which are correctly identified as such (i.e. the percentage of sick people who are identified as having the condition); and the specificity measures the proportion of negatives which are correctly identified (i.e. the percentage of well people who are identified as not having the condition).

For any test, there is usually a trade-off between each measure. For example in a manufacturing setting in which one is testing for faults, one may be willing to risk discarding functioning components (low specificity), in order to increase the chance of identifying nearly all faulty components (high sensitivity). This trade-off can be represented graphically using a ROC curve (receiver operating characteristic).

$$sensitivity = \frac{number of True Positives}{number of True Positives + number of False Negatives}$$

A sensitivity of 100% means that the test recognizes all sick people as such. Thus in a high sensitivity test, a negative result is used to rule out the disease.

Sensitivity alone does not tell us how well the test predicts other classes (that is, about the negative cases). In the binary classification, as illustrated above, this is the corresponding specificity test, or equivalently, the sensitivity for the other classes.

$$specificity = \frac{number of True Negatives}{number of True Negatives + number of False Positives}$$

The accuracy of a measurement system is the degree of closeness of measurements of a quantity to its actual (true) value. The precision of a measurement system, also called reproducibility or repeatability, is the degree to which repeated measurements under unchanged conditions show the same results.

Accuracy is also used as a statistical measure of how well a binary classification test correctly identifies or excludes a condition. That is, the accuracy is the proportion of true results (both true positives and true negatives) in the population. It is a parameter of the test:

$$accuracy = \frac{number of true positives + number of true negatives}{\begin{matrix}number of true positives + false positives + \\ false negatives + true negatives\end{matrix}}$$

An accuracy of 100% means that the measured values are exactly the same as the given values.

miRNA Classifier of the Present Invention

The miRNA classifiers according to the present invention are the relationships between sets of input variables, i.e. the miRNA expression in a blood sample of an individual, and discrete output variables, i.e. distinction between e.g. a cancerous and non-cancerous condition of the pancreas. Thus, the classifier assigns a given sample to a given class with a given probability.

Distinction, differentiation or characterisation of a sample is used herein as being capable of predicting with a high sensitivity and specificity if a given sample of unknown diagnosis belongs to one of two classes (two-way classifier).

In one aspect, the miRNA classifier is a two-way classifier capable of predicting with an adequate sensitivity and specificity if a given whole blood sample of a subject/patient with unknown diagnosis belongs to the class of patients with pancreatic carcinoma (pancreatic and and/or ampullary adenocarcinoma) or the class of subjects with normal pancreas (NP; alternatively healthy subjects HS) and/or chronic pancreatitis (CP), wherein said miRNA classifier comprises or consists of one or more miRNAs in whole blood selected from the group consisting of or comprising
a. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
b. let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-505, miR-508.3p, miR-539, miR-582.3p, miR-625, miR-628.3p, miR-636, miR-769.5p, miR-885.5p, miR-935 and miR-941; and/or
c. let-7g, miR-26b, miR-30b, miR-31, miR-34a, miR-122, miR-126*, miR-145, miR-150, miR-223, miR-505, miR-636 and miR-885.5p; and/or
d. miR-122, miR-34a, miR-145, miR-636, miR-223, miR-26b, miR-885.5p, miR-150, miR-126* and miR-505; and/or
e. miR-150, miR-30b, miR-145 and miR-223; and/or
f. miR-150, miR-636, miR-145 and miR-223.

In one aspect, the miRNA classifier is a two-way classifier capable of predicting with an adequate sensitivity and specificity if a given plasma sample of a subject/patient with unknown diagnosis belongs to the class of patients with pancreatic carcinoma (pancreatic and/or ampullary adenocarcinoma) or the class of subjects with normal pancreas and/or chronic pancreatitis, wherein said miRNA classifier comprises or consists of one or more miRNAs in plasma selected from the group consisting of or comprising of miR-let7b, miR-10b*, miR-17, miR-19a, miR-19b, miR-20b, miR-24, miR-27a, miR-30d, miR-93, miR-106a, miR-126, miR-126*, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305.

In one aspect, the miRNA classifier is a two-way classifier capable of predicting with an adequate sensitivity and specificity if a given serum sample from a subject/patient of unknown diagnosis belongs to the class of patients with pancreatic carcinoma (pancreatic and/or ampullary adenocarcinoma) or the class of subjects with normal pancreas and/or chronic pancreatitis, wherein said miRNA classifier comprises or consists of one or more miRNAs in serum selected from the group consisting of or comprising
a. miR-25, miR-26a, miR-26b, miR-27a, miR-29c, miR-30a-5p, miR-106a, miR-195, miR-212, miR-320, miR-323-3p, miR-345, miR-483-5p, miR-485-3p, miR-590-5p, miR-618, miR-638, and miR-645.
b. let-7b, miR-24, miR-25, miR-26a, miR-26b, miR-27a, miR-29c, miR-30a-5p, miR-30e.3p, miR-106a, miR-148a, miR-185, miR-191, miR-195, miR-212, miR-320, miR-323-3p, miR-345, miR-483-5p, miR-485-3p, miR-590-5p, miR-618, miR-638, and miR-645; and/or
c. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645; or
d. miR-16, miR-27a, miR-30a.5p, miR-20a, miR-25 and miR-483.5p; and/or
e. miR-16, miR-27a, miR-30a.5p, miR-323.3p, miR-20a, miR-29c and miR-483.5p; and/or
f. miR-16, miR-24, miR-27a, miR-30a.5p, miR-485.3p, miR-20a, miR-25, miR-29c, miR-99a, miR-345, miR-483.5p and miR-618; and/or
g. miR-16, miR-24, miR-27a, miR-30a.5p, miR-323.3p, miR-20a, miR-25, miR-29c and miR-483.5p; and/or
h. miR-24, miR-27a, miR-323.3p, miR-20a and miR-483.5p; and/or
i. miR-16, miR-27a, miR-25, miR-29c and miR-483.5p; and/or
j. miR-16, miR-18a, miR-24, miR-27a, miR-30a.5p, miR-323.3p, miR-20a, miR-25, miR-29c, miR-30e.3p, miR-99a, miR-345 and miR-483.5p; and/or
k. miR-16, miR-18a, miR-24, miR-27a, miR-30a.5p, miR-323.3p, miR-20a, miR-25, miR-29c, miR-191, miR-345 and miR-483.5p.
l. let-7b, miR-16, miR-18a, miR-20a, miR-24, miR-25, miR-26a, miR-26b, miR-27a, miR-29c, miR-30a-5p, miR-30e.3p, miR-99a, miR-106a, miR-181a, miR-185, miR-191, miR-195, miR-323-3p, miR-345, miR-483-5p, miR-485-3p, miR-590-5p and miR-618; or
m. miR-16, miR-18a, miR-24, miR-26a, miR-26b, miR-27a, miR-30a.5p, miR-30e.3p, miR-99a, miR-106a, miR-323.3p, miR-20a, miR-25, miR-29c, miR-181a, miR-185, miR-191, miR-195, miR-345, miR-483-5p, miR-485-3p and miR-590.5p; or
n. miR-16, miR-18.a, miR-20a, miR-24, miR-25, miR-27a, miR-29c, miR-30a.5p, miR-191, miR-323.3p, miR-345, and miR-483.5p.
o. miR-16, miR-20a, miR-25, miR-27a, miR-30a.5p, miR-99a, miR-195, miR-483.5p and miR-618
p. miR-16, miR-20a, miR-24, miR-25, miR-30a.5p, miR-30e.3p, miR-106a, miR-195, miR-345, and miR-483.5p
q. miR-16, miR-20a, miR-24, miR-25, miR-27a, miR-29c, miR-30a.5p, miR-30e.3p, miR-185, miR-195, miR-323.3p, miR-345, miR-483.5p

In a particular embodiment, the miRNA classifier is a two-way classifier capable of predicting with an adequate sensitivity and specificity if a given sample of a subject/patient with unknown diagnosis belongs to the class of either patients with pancreatic carcinoma (pancreatic and/or ampullary adenocarcinoma) or to the class of subjects with normal pancreas and/or chronic pancreatitis.

The miRNA classifiers according to the present invention are the relationships between sets of input variables, i.e. the miRNA expression in a sample of a patient with a diagnosis that belongs to the combined class of either patients with pancreatic carcinoma and ampullary adenocarcinoma, and discrete output variables, i.e. distinction between two conditions e.g. poor or good survival. Thus, the classifier assigns a given sample to a given class with a given probability.

Distinction, differentiation or characterisation of a sample is used herein as being capable of predicting with a high sensitivity and specificity if a given sample of unknown prognosis belongs to one of two classes (two-way classifier).

In one aspect, the miRNA classifier is a two-way classifier capable of predicting with an adequate sensitivity and specificity if a given blood sample of a patient with a diagnosis that belongs to the combined class of either patients with pancreatic carcinoma and ampullary adenocarcinoma and has a unknown prognosis has a certain probability of being associated with a specific predicted survival, wherein said miRNA classifier comprises or consists of one or more miRNAs in blood selected from the group consisting of miR-let7g, miR-16, miR-19b, miR-20a, miR-21, miR-27a, miR-30b, miR-30e-3p, miR-99b, miR-100, miR-146a, miR-146b-5p, miR-148a, miR-181a, miR-185, miR-320, miR-328, miR-331-3p, miR-511, miR-362-3p, miR-511, miR-758, miR-1238, miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636.

In another aspect, the miRNA classifier is a two-way classifier capable of predicting with an adequate sensitivity and specificity if a given serum sample of a patient with a diagnosis that belongs to the combined class of either patients with pancreatic carcinoma and ampullary adenocarcinoma and has a unknown prognosis has a certain probability of being associated with a specific predicted survival, wherein said miRNA classifier comprises or consists of one or more miRNAs in serum selected from the group consisting of miR-let7g, miR-16, miR-19b, miR-20a, miR-21, miR-27a, miR-30b, miR-30e-3p, miR-99b, miR-100, miR-146a, miR-146b-5p, miR-148a, miR-181a, miR-185, miR-320, miR-328, miR-331-3p, miR-511, miR-362-3p, miR-758 and miR-1238.

In another aspect, the miRNA classifier is a two-way classifier capable of predicting with an adequate sensitivity and specificity if a given whole blood sample of a patient with a diagnosis that belongs to the combined class of either patients with pancreatic carcinoma and ampullary adenocarcinoma and has a unknown prognosis has a certain probability of being associated with a specific predicted survival, wherein said miRNA classifier comprises or consists of one or more miRNAs selected from the group consisting of miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636.

For any of the above embodiments relating to prognosis the individual may be a patient who has undergone surgery for the pancreatic cancer or an individual who has been deemed unfit for surgery. The prognosis may be given for an individual with a solid or encapsulated tumor or for a patient with metastatic pancreatic cancer. Thus the prognosis may be given based on a blood sample, such as a whole blood, serum or plasma sample (or a combination hereof) taken from the individual after he/she has undergone surgery.

Likewise for any of the above embodiments relating to diagnosis the individual may have an unknown diagnosis, or may be an individual with a diagnosis, such as a diagnosis of pancreatic cancer (that thus may be confirmed or denied) or the individual may previously have suffered from pancreatic cancer and is checked for relapse and the sample analysed may be a blood sample such as a whole blood, serum or plasma sample or a combination of any of these.

Said specific predicted survival may be expressed as the probability for surviving at 3-months, 6-months, 9-months, 12-months/1-year, 2-years, 3-years, 4-years, 5-years, 6-years, 7-years, 8-years, 9-years or 10-years; calculated from time of diagnosis, time of surgery or time of analysis/evaluation.

Platt's probabilistic outputs for Support Vector Machines (Platt, J. in Smola, A. J, et al. (eds.) Advances in large margin classifiers. Cambridge, 2000; incorporated herein by reference) is useful for applications that require posterior class probabilities. Also incorporated by reference herein is Platt J. Advances in Large Classifiers. Cambridge, Mass.: MIT Press, 1999.

The output of the two-way miRNA classifier is given as a probability of belonging to either class of between 0-1 (prediction probability). If the value for a sample is 0.5, no prediction is made. A number or value of between 0.51 to 1.0 for a given sample means that the sample is predicted to belong to the class in question, e.g. normal pancreas (NP); and the corresponding value of 0.0 to 0.49 for the second class in question, e.g. pancreatic cancer (PC), means that the sample is predicted not to belong to the class in question.

In one embodiment, the prediction probabilities for a sample to belong to a certain class is a number falling in the range of from 0 to 1, such as from 0.0 to 0.1, for example 0.1 to 0.2, such as 0.2 to 0.3, for example 0.3 to 0.4, such as 0.4 to 0.49, for example 0.5, such as 0.51 to 0.6, for example 0.6 to 0.7, such as 0.7 to 0.8, for example 0.8 to 0.9, such as 0.9 to 1.0.

In one embodiment, the prediction probability for a sample to belong to the normal pancreas (NP) class is a number falling in the range of from 0 to 0.49, 0.5 or from 0.51 to 1.0. In another embodiment, the prediction probability for a sample to belong to the pancreatic cancer (PC) class is a number between from 0 to 0.49, 0.5 or between from 0.51 to 1.0.

The classifier for serum samples according to the present invention may in one embodiment consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs, such as 21 miRNAs, for example 22 miRNAs, such as 23 miRNAs, for example 24 miRNAs, for example 22 miRNAs, such as 23 miRNAs, for example 24 miRNAs, for example 25 miRNAs, such as 26 miRNAs, for example 27 miRNAs, for example 28 miRNAs, such as 29 miRNAs, for example 30 miRNAs, for example 31 miRNAs, such as 32 miRNAs, for example 33 miRNAs, such as 34 miRNAs selected from the group consisting of let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645.

The classifier for whole blood samples according to the present invention may in one embodiment consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs selected from the group consisting of let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941.

The classifier for plasma samples according to the present invention may in one embodiment consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs, such as 21 miRNAs, for example 22 miRNAs, such as 23 miRNAs, for example 24 miRNAs, such as 25 miRNAs selected from the group consisting of miR-let7b, miR-10b*, miR-17, miR-19a, miR-19b, miR-20b, miR-24, miR-27a, miR-30d, miR-93, miR-106a, miR-126, miR-126*, miR-1395p, miR-1403p, miR-1405p, miR-146b5p, miR-146a, miR-151-3p, miR-151-5p, miR-152, miR-186, miR-191, miR-197, miR-223, miR-320, miR-320b, miR-323-3p, miR-3243p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305.

In one embodiment, the two-way miRNA classifier further comprises one or more additional miRNAs selected from the deregulated miRNA biomarkers as disclosed herein above. In one embodiment, the two-way miRNA classifiers further comprises one or more additional miRNAs, such as 1 additional miRNA, for example 2 additional miRNAs, such as 3 additional miRNAs, for example 4 additional miRNAs, such as 5 additional miRNAs, for example 6 additional miRNAs, such as 7 additional miRNAs, for example 8 additional miRNAs, such as 9 additional miRNAs, for example 10 additional miRNAs, such as 11 additional miRNAs, for example 12 additional miRNAs, such as 13 additional miRNAs, for example 14 additional miRNAs, such as 15 additional miRNAs, for example 16 additional miRNAs, such as 17 additional miRNAs, for example 18 additional miRNAs, such as 19 additional miRNAs, for example 20 additional miRNAs selected from the deregulated miRNA biomarkers as disclosed herein above.

In an embodiment, an alteration of the expression profile or signature of one or more of the miRNAs of the two-way miRNA classifier according to the present invention is associated with the sample being classified as pancreatic cancer. In an embodiment, an alteration of the expression profile or signature of one or more of the miRNAs of the two-way miRNA classifier is associated with the sample from the subject being classified as having a normal pancreas and/or chronic pancreatitis.

The miRNA classifiers disclosed herein in a particular embodiment has a sensitivity of malignancy of pancreatic cancer as at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has an accuracy for pancreatic cancer of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a specificity for pancreatic cancer of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a negative predictive value for malignancies of pancreatic cancer of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a positive predictive value for malignancies of pancreatic cancer of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a positive predictive value or a negative predictive value for malignancies of pancreatic cancer of between 80-85%, such as 85-90%, for example 90-95%, such as 95-96%, for example 96-97%, such as 97-98%, for example 98-99%, such as 99-100%.

Methods for Diagnosis and/or Prognosis Employing the miRNA Classifier and/or Biomarkers of the Present Invention

It is an object of the present invention to provide a method for diagnosing if an individual has, or is at risk of developing, pancreatic carcinoma, and/or a method for giving/predicting a prognosis for the survival of the individual with pancreatic carcinoma, said method comprising measuring the level of one or more miRNA in a blood sample obtained from said individual, wherein the one or more miRNA is selected from the group consisting of or comprising:
a) in a serum sample for diagnosis
   a. miR-25, miR-26a, miR-26b, miR-27a, miR-29c, miR-30a-5p, miR-106a, miR-195, miR-212, miR-320, miR-323-3p, miR-345, miR-483-5p, miR-485-3p, miR-590-5p, miR-618, miR-638, and miR-645
   b. let-7b, miR-24, miR-25, miR-26a, miR-26b, miR-27a, miR-29c, miR-30a-5p, miR-30e.3p, miR-106a, miR-148a, miR-185, miR-191, miR-195, miR-212, miR-320, miR-323-3p, miR-345, miR-483-5p, miR-485-3p, miR-590-5p, miR-618, miR-638, and miR-645; and/or
   c. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645; or
   d. miR-16, miR-27a, miR-30a.5p, miR-20a, miR-25 and miR-483.5p; and/or
   e. miR-16, miR-27a, miR-30a.5p, miR-323.3p, miR-20a, miR-29c and miR-483.5p; and/or
   f. miR-16, miR-24, miR-27a, miR-30a.5p, miR-485.3p, miR-20a, miR-25, miR-29c, miR-99a, miR-345, miR-483.5p and miR-618; and/or
   g. miR-16, miR-24, miR-27a, miR-30a.5p, miR-323.3p, miR-20a, miR-25, miR-29c and miR-483.5p; and/or
   h. miR-24, miR-27a, miR-323.3p, miR-20a and miR-483.5p; and/or
   i. miR-16, miR-27a, miR-25, miR-29c and miR-483.5p; and/or
   j. miR-16, miR-18a, miR-24, miR-27a, miR-30a.5p, miR-323.3p, miR-20a, miR-25, miR-29c, miR-30e.3p, miR-99a, miR-345 and miR-483.5p; and/or
   k. miR-16, miR-18a, miR-24, miR-27a, miR-30a.5p, miR-323.3p, miR-20a, miR-25, miR-29c, miR-191, miR-345 and miR-483.5p; and/or
   l. let-7b, miR-16, miR-18a, miR-20a, miR-24, miR-25, miR-26a, miR-26b, miR-27a, miR-29c, miR-30a-5p, miR-30e.3p, miR-99a, miR-106a, miR-181a, miR-185, miR-191, miR-195, miR-323-3p, miR-345, miR-483-5p, miR-485-3p, miR-590-5p and miR-618; or
   m. miR-16, miR-18a, miR-24, miR-26a, miR-26b, miR-27a, miR-30a.5p, miR-30e.3p, miR-99a, miR-106a, miR-323.3p, miR-20a, miR-25, miR-29c, miR-181a, miR-185, miR-191, miR-195, miR-345, miR-483-5p, miR-485-3p and miR-590.5p; or
   n. miR-16, miR-18.a, miR-20a, miR-24, miR-25, miR-27a, miR-29c, miR-30a.5p, miR-191, miR-323.3p, miR-345, and miR-483.5p.
   o. miR-16, miR-20a, miR-25, miR-27a, miR-30a.5p, miR-99a, miR-195, miR-483.5p and miR-618
   p. miR-16, miR-20a, miR-24, miR-25, miR-30a.5p, miR-30e.3p, miR-106a, miR-195, miR-345, and miR-483.5p
   q. miR-16, miR-20a, miR-24, miR-25, miR-27a, miR-29c, miR-30a.5p, miR-30e.3p, miR-185, miR-195, miR-323.3p, miR-345, miR-483.5p
b) in a whole blood sample for diagnosis
   a. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
   b. let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-505, miR-508.3p, miR-539, miR-582.3p, miR-625, miR-628.3p, miR-636, miR-769.5p, miR-885.5p, miR-935 and miR-941; and/or
   c. let-7g, miR-26b, miR-30b, miR-31, miR-34a, miR-122, miR-126*, miR-145, miR-150, miR-223, miR-505, miR-636 and miR-885.5p; and/or
   d. miR-122, miR-34a, miR-145, miR-636, miR-223, miR-26b, miR-885.5p, miR-150, miR-126* and miR-505; and/or
   e. miR-150, miR-30b, miR-145 and miR-223.
   f. miR-150, miR-636, miR-145 and miR-223;
c) in a plasma sample for diagnosis miR-let7b, miR-10b*, miR-17, miR-19a, miR-19b, miR-20b, miR-24, miR-27a, miR-30d, miR-93, miR-106a, miR-126, miR-126*, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305;
d) in a serum sample for prognosis of miR-19b, miR-27a, miR-30b, miR-30e-3p, miR-99b, miR-100, miR-181a, miR-185, miR-331-3p, miR-511, miR-362-3p, miR-758 and miR-1238;
e) in a whole blood sample for prognosis miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636,
   wherein the miRNA level, and/or the difference in the miRNA level, of at least one of said miRNAs compared to a control is indicative of said individual having, or being at risk of developing, pancreatic carcinoma and/or giving a prognosis for the survival of said individual.

In one embodiment there is provided a method for diagnosing if an individual has, or is at risk of developing, pancreatic carcinoma. In one embodiment, said individual is suspected of having pancreatic carcinoma.

In one embodiment there is provided a method for giving/predicting a prognosis for the survival of the individual with pancreatic carcinoma, i.e. already having been diagnosed with pancreatic carcinoma.

In one embodiment, the method comprises measuring the level of miRNAs in a blood sample obtained from said individual, wherein all miRNAs included in each of the individual groups as identified herein above are included in the method (i.e. a) a.-q.; b) a.-e, c), d) or e)).

In one embodiment, the blood sample is a whole blood sample used for diagnosis, and the miRNAs comprise at least or consist of the group consisting of miR-145, miR-150 and miR-223.

In one embodiment, the blood sample is a whole blood sample used for diagnosis, and the miRNAs are selected from the group consisting of Let-7g, miR-26b, miR-30b, miR-31, miR-34a, miR-122, miR-126*, miR-145, miR-150, miR-223, miR-505, miR-636 and miR-885.5p; wherein said miRNAs represent the miRs chosen for validation.

In one embodiment, the blood sample is a serum sample used for diagnosis, and the miRNAs are selected from the group consisting of let-7b, miR-16, miR-18a, miR-20a, miR-25, miR-26a, miR-26b, miR-27a, miR-29c, miR-30a-5p, miR-30e.3p, miR-99a, miR-106a, miR-181a, miR-185, miR-191, miR-195, miR-323-3p, miR-345, miR-483-5p, miR-485-3p, miR-590-5p and miR-618; wherein said miRNAs represent the significant miRs of table 11 (Training, control=NP and CP).

In one embodiment, the blood sample is a serum sample used for diagnosis, and the miRNAs are selected from the group consisting of miR-16, miR-18a, miR-24, miR-26a, miR-26b, miR-27a, miR-30a.5p, miR-30e.3p, miR-99a, miR-106a, miR-323.3p, miR-20a, miR-25, miR-29c, miR-181a, miR-185, miR-191, miR-195, miR-345, miR-483-5p, miR-485-3p and miR-590.5p; wherein said miRNAs represent the significant miRs of table 12 (Training, control=NP).

In one embodiment, the blood sample is a serum sample used for diagnosis, and the miRNAs are selected from the group consisting of miR-16, miR-18.a, miR-20a, miR-24, miR-25, miR-27a, miR-29c, miR-30a.5p, miR-191, miR-323.3p, miR-345, and miR-483.5p; wherein said miRNAs represent the 12 miRs included in the various diagnostic indexes of table 13.

In one embodiment, the blood sample is a serum sample used for diagnosis, and the miRNAs are selected from the group consisting of miR-16, miR-20a, miR-25, miR-27a, miR-30a.5p, miR-99a, miR-195, miR-483.5p and miR-618; wherein said miRNAs represent the miRs that are independent biomarkers for diagnosis of PC compared to HS and CP combined (table 11).

In one embodiment, the blood sample is a serum sample used for diagnosis, and the miRNAs are selected from the group consisting of miR-16, miR-20a, miR-24, miR-25, miR-30a.5p, miR-30e.3p, miR-106a, miR-195, miR-345, and miR-483.5p; wherein said miRNAs represent the miRs that are independent biomarkers for diagnosis of PC compared to HS (table 12).

In one embodiment, the blood sample is a serum sample used for diagnosis, and the miRNAs are selected from the group consisting of miR-16, miR-20a, miR-24, miR-25, miR-27a, miR-29c, miR-30a.5p, miR-30e.3p, miR-185, miR-195, miR-323.3p, miR-345, miR-483.5p; wherein said miRNAs represent the miRs that met the 0.05 significance level in both the “Discovery Study” and “Training Study”, and thus were tested in the Validation study.

For the prognosis method, said specific predicted survival may be expressed as the probability for surviving at 3-months, 6-months, 9-months, 12-months/1-year, 2-years, 3-years, 4-years, 5-years, 6-years, 7-years, 8-years, 9-years or 10-years; calculated from time of diagnosis, time of surgery or time of analysis/evaluation.

In is understood that said difference in miRNA expression level in a preferred embodiment is a relative difference between said miRNA's expression levels.

In one embodiment, said method further comprises the step of obtaining a blood sample from an individual, by any means as disclosed herein elsewhere.

In one embodiment, said method further comprises the step of collecting/providing/obtaining a blood sample, such as a whole blood, serum and/or plasma sample, from an individual, by any means as disclosed herein elsewhere. In a particular embodiment, said blood sample is collected/provided/obtained in a container which has means for stabilising the RNA of the blood sample, including the miRNA, for example by providing for decreased RNA degradation. In one embodiment, such device is a PAXgene Blood RNA Tubes (Qiagen, available from BD, cat. no. 762165; see www.PreAnalytiX.com). Such tubes are suitable for blood collection, stabilization, and transport, and maintain RNA in the collected blood stable for at least 3 days at room temperature (at least 50 months at −20° C.), thus further facilitating on-site collection without the immediate need for purification or storage on ice.

In one embodiment, said method further comprises the step of extracting RNA from a blood sample, such as a whole blood, serum and/or plasma sample collected from an individual, by any means as disclosed herein elsewhere.

In one embodiment, said method further comprises the step of determining the miRNA expression levels in a blood sample from an individual, by any means as disclosed herein elsewhere.

In one embodiment, said method further comprises the step of comparing and/or correlating the miRNA expression level of at least one of said miRNAs to a predetermined control level.

In one embodiment, said method further comprises the step of determining if said individual has, or is at risk of developing, pancreatic carcinoma.

In one embodiment, said miRNA expression level is altered as compared to the expression level in a control. Said control is a normalized healthy sample, i.e. derived from the results obtained from blood samples of healthy individuals. Alternatively said control is a normalized sample of both healthy individuals and individuals/patients with chronic pancreatitis.

In one embodiment, said pancreatic carcinoma is pancreatic adenocarcinoma. In another embodiment, said pancreatic carcinoma is ampullary adenocarcinoma. In a further embodiment, said pancreatic carcinoma comprises both pancreatic adenocarcinoma and ampullary adenocarcinoma.

In a further embodiment, any of the above-mentioned methods may be is used in combination with at least one additional diagnostic and/or prognostic method. Said at least one additional diagnostic method may in one embodiment be selected from the group consisting of CT (X-ray computed tomography), MRI (magnetic resonance imaging), Scintillation counting, Blood sample analysis, Ultrasound imaging, Cytology, Histology and Assessment of risk factors. These are described herein above. In one embodiment, said at least one additional diagnostic method improves the sensitivity and/or specificity of the combined diagnostic outcome.

In one embodiment, the method for predicting a prognosis for the survival of the individual with pancreatic carcinoma according to the present invention further comprises the step of determining the probability for said individual with pancreatic carcinoma of surviving for the indicated time period. Said probability of surviving for a certain time period may be in the range of 0.01 to 0.1, such as 0.1 to 0.2, for example 0.2 to 0.3, such as 0.3 to 0.4, for example 0.4 to 0.5, such as 0.5 to 0.6, for example 0.6 to 0.7, such as 0.7 to 0.8, for example 0.8 to 0.85, such as 0.85 to 0.9, for example 0.9 to 0.91, such as 0.91 to 0.92, for example 0.92 to 0.93, such as 0.93 to 0.94, for example 0.94 to 0.95, such as 0.95 to 0.96, for example 0.96 to 0.97, such as 0.97 to 0.98, for example 0.98 to 0.99, such as 0.99 to 1.0.

In one embodiment, said time period may be expressed as 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability.

Said time period may be calculated starting from time of diagnosis, time of surgery or time of analysis/evaluation.

In one particular embodiment, the step of determining the probability for said individual with pancreatic carcinoma of surviving for an indicated time period is performed by employing a nomogram. A nomogram, nomograph, or abac is a graphical calculating device, a two-dimensional diagram designed to allow the approximate graphical computation of a function. Defining alternatively, a nomogram is a (two-dimensionally) plotted function with n parameters, from which, knowing n-1 parameters, the unknown one can be read, or fixing some parameters, the relationship between the unfixed ones can be studied.

In one embodiment, said method further comprises the step of correlating the miRNA expression level of at least one of said miRNAs to a predetermined reference level.

In one embodiment, said miRNA expression level is altered as compared to the expression level in a reference sample. Said reference sample may in one embodiment be a sample from a patient with a known estimated prognosis.

In one embodiment, the prognosis as defined herein is expressed as a probability of surviving a certain time period expressed in months or years. Said time period may be defined as 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability.

### Diagnostic Index

It is a further aspect of the present invention to provide diagnostic indices or indexes for diagnosing pancreatic cancer based on the miRNA expression levels in a blood sample from an individual. Said individual may or may not be suspected of having pancreatic cancer.

Technical variation can be cancelled out by having a balanced sum of signs with plusses for miRNAs with OR>1 and minuses for miRNAs with OR<1.

The Diagnostic index (DI) may be calculated by addition (‘+’) and/or subtraction (‘−’) of the expression values for two or more miRNAs. In a further embodiment each of said miRNA expression values may be further weighed by multiplication with a factor, wherein said factor may be below 1 or above 1.

It follows that in one embodiment, a diagnosis may be made based on a whole blood sample by determining the expression levels of two or more miRNAs in said whole blood sample, and correlating their expression to one another.

The present invention provides in one embodiment provides a diagnostic index for determining if an individual has, or is at risk of developing, pancreatic carcinoma, comprising measuring in a whole blood sample the expression level of miR-150, miR-30b, miR-145 and miR-223, and correlating the expression levels with the following formula:

miR-150−miR-30b−miR-145+miR-223

The present invention provides in one embodiment provides a diagnostic index for determining if an individual has, or is at risk of developing, pancreatic carcinoma, comprising measuring in a whole blood sample the expression level of miR-150, miR-636, miR-145 and miR-223, and correlating the expression levels with the following formula:

miR-150−miR-636−miR-145+miR-223

The present invention provides in one embodiment provides a diagnostic index for determining if an individual has, or is at risk of developing, pancreatic carcinoma, comprising measuring in a whole blood sample the expression level of miR-122, miR-34a, miR-145, miR-636, miR-223, miR-26b, miR-885.5p, miR-150, miR-126* and miR-505, and correlating the expression levels with the following formula:

6.9275−(0.2134×miR-122)−(0.3560×miR-34a)−(0.8577×miR-145)+(1.0043×miR-636)−(0.6725×miR-223)+(0.7018×miR-26b)−(0.3233×miR-885.5p)+(1.1304×miR-150)−(0.2204×miR-126*)−(0.1730×miR-505).

It follows that in one embodiment, a diagnosis may be made based on a serum sample by determining the expression levels of two or more miRNAs in said serum sample, and correlating their expression to one another.

The present invention provides in one embodiment provides a diagnostic index for determining if an individual has, or is at risk of developing, pancreatic carcinoma, comprising measuring in a serum sample the expression level of miR-16, miR-27a, miR-30a.5p, miR-323.3p, miR-20a, miR-29c and miR-483.5p, and correlating the expression levels with the following formula:

+miR-16+miR-27a+miR-30a.5p+miR-323.3p−miR-20a−miR-29c−miR-483.5p

The present invention provides in one embodiment provides a diagnostic index for determining if an individual has, or is at risk of developing, pancreatic carcinoma, comprising measuring in a serum sample the expression level of miR-16, miR-27a, miR-25, miR-29c and miR-483.5p, and correlating the expression levels with the following formula:

+miR-16+miR-27a−miR-25−miR-29c−miR-483.5p

The present invention provides in one embodiment provides a diagnostic index for determining if an individual has, or is at risk of developing, pancreatic carcinoma, comprising measuring in a serum sample the expression level of miR-16, miR-24, miR-27a, miR-30a.5p, miR-323.3p, miR-20a, miR-25, miR-29c and miR-483.5p, and correlating the expression levels with the following formula:

+(0.41×miR-16)+(0.56×miR-24)+(0.25×miR-27.a)+(0.55×miR-30a.5p)+(0.18×miR-323.3p)−(0.44×miR-20a)−(0.37×miR-25)−(0.20×miR-29c)−(0.71×miR-483.5p).

The present invention provides in one embodiment provides a diagnostic index for determining if an individual has, or is at risk of developing, pancreatic carcinoma, comprising measuring in a serum sample the expression level of miR-16, miR-18a, miR-24, miR-27a, miR-30a.5p, miR-323.3p, miR-20a, miR-25, miR-29c, miR-191, miR-345 and miR-483.5p, and correlating the expression levels with the following formula:

+(1.92×miR-16)+(0.12×miR-18.a)+(1.38×miR-24)+(0.67×miR-27a)+(0.60×miR-30a.5p)+(0.36×miR-323.3p)−(1.37×miR-20a)−(0.61×miR-25)−(0.55×miR-29c)−(0.37×miR-191)−(0.44×miR-345)−(1.03×miR-483.5p).

The present invention provides in one embodiment provides a diagnostic index for determining if an individual has, or is at risk of developing, pancreatic carcinoma, comprising measuring in a serum sample the expression level of miR-16, miR-27a, miR-30a.5p, miR-20a, miR-25 and miR-483.5p, and correlating the expression levels with the following formula:

+miR-16+miR-27a+miR-30a.5p−miR-20a−miR-25−miR-483.5p

The present invention provides in one embodiment provides a diagnostic index for determining if an individual has, or is at risk of developing, pancreatic carcinoma, comprising measuring in a serum sample the expression level of miR-16, miR-24, miR-27a, miR-30a.5p, miR-485.3p, miR-20a, miR-25, miR-29c, miR-99a, miR-345, miR-483.5p and miR-618, and correlating the expression levels with the following formula:

+(0.43×miR-16)+(0.62×miR-24)+(0.33×miR-27.a)+(0.68×miR-30a.5p)+(0.08×miR-485.3p)−(0.46×miR-20a)−(0.34×miR-25)−(0.22×miR-29c)−(0.06×miR-99a)−(0.20×miR-345)−(0.65×miR-483.5p)−(0.11×miR-618)

The present invention provides in one embodiment provides a diagnostic index for determining if an individual has, or is at risk of developing, pancreatic carcinoma, comprising measuring in a serum sample the expression level of miR-24, miR-27a, miR-323.3p, miR-20a and miR-483.5p, and correlating the expression levels with the following formula:

+miR-24+miR-27a+miR-323.3p−miR-20a−miR-483.5p

The present invention provides in one embodiment provides a diagnostic index for determining if an individual has, or is at risk of developing, pancreatic carcinoma, comprising measuring in a serum sample the expression level of miR-16, miR-18a, miR-24, miR-27a, miR-30a.5p, miR-323.3p, miR-20a, miR-25, miR-29c, miR-30e.3p, miR-99a, miR-345 and miR-483.5p, and correlating the expression levels with the following formula:

+(1.96×miR-16)+(0.15×miR-18.a)+(1.23×miR-24)+(0.64×miR-27a)+(0.70×miR-30a.5p)+(0.39×miR-323.3p)−(1.23×miR-20a)−(0.80×miR-25)−(0.53×miR-29c)−(0.25×miR-30e.3p)−(0.11×miR-99a)−(0.40×miR-345)−(0.95×miR-483.5p)

A Model for Predicting a Diagnosis and/or Prognosis by Employing the miRNA Classifier of the Present Invention

In one aspect, the present invention relates to a model for predicting the diagnosis of an individual, comprising
i) providing a set of input data to the miRNA classifier according to the present invention, and
ii) determining if said individual has a condition selected from pancreatic carcinoma (pancreatic and/or ampullary adenocarcinoma) and chronic pancreatitis.

In one embodiment, said input data comprises or consists of the miRNA expression profile of one or more of the following miRNAs:
a) in a serum sample for diagnosis
   let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645, or
b) in a whole blood sample for diagnosis let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941, or
c) in a plasma sample for diagnosis miR-let7b, miR-10b*, miR-17, miR-19a, miR-19b, miR-20b, miR-24, miR-27a, miR-30d, miR-93, miR-106a, miR-126, miR-126*, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305;
d) in a serum sample for prognosis of miR-19b, miR-27a, miR-30b, miR-30e-3p, miR-99b, miR-100, miR-181a, miR-185, miR-331-3p, miR-511, miR-362-3p, miR-758 and miR-1238;
e) in a whole blood sample for prognosis miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636.

In a further embodiment, the model according to the present invention further comprises one or more additional miRNAs selected from the deregulated miRNA biomarkers disclosed herein.

### Sample Type

The sample according to the present invention is extracted from an individual and used for miRNA profiling for the subsequent diagnosis of a condition of the pancreas or the evaluation of a prognosis of a pancreas cancer patient. In one embodiment, said individual is suspected of having pancreatic cancer.

The sample may be collected from an individual or a cell culture, preferably an individual. The individual may be any animal, such as a mammal, including human beings. In a preferred embodiment, the individual is a human being.

Collection of blood samples may for example be made by finger stick, heel stick, or venepuncture (blood sampling of venous blood).

In a particular embodiment, the sample is a blood sample, such as a blood sample drawn from a human being. The blood sample may comprise arterial, capillary and/or venous blood; preferably the sample is of venous blood. Venous blood may be collected by e.g. finger stick, heel stick or venepuncture. The sample may be a whole blood sample, taken and optionally stored prior to analysis as is customary in the art. Alternatively the blood sample may be separated to yield plasma and/or serum and thus the sample may be a plasma sample or a serum sample. To separate the blood, the blood is centrifuged to remove cellular components. Anti-coagulated blood yields plasma containing fibrinogen and clotting factors. Coagulated blood (clotted blood) yields serum without fibrinogen, although some clotting factors remain. The blood sample may be mixed with e.g. EDTA or Lithium Heparin to prevent clotting, or other factors to prevent the degradation of RNA and specifically miRNA in the samples. Pre-prepared sampling devices may be used for storage of the samples, e.g. pre-prepared tubes with EDTA or PAXgene Blood RNA Tubes (QIAGEN) for stabilization of RNA.

The PAXgene Blood RNA System (QIAGEN) consists of a blood collection tube (PAXgene Blood RNA Tube) and nucleic acid purification kit (PAXgene Blood RNA Kit). It is intended for the collection, storage, and transport of blood and stabilization of intracellular RNA in a closed tube and subsequent isolation and purification of intracellular RNA from whole blood for RT-PCR used in molecular diagnostic testing.

Thus the samples of the present invention are blood samples, such as whole blood collected in PAXgene Blood RNA Tubes, serum samples and/or plasma samples.

### Sample Collection

Sample collection may be performed as is customary in the art by drawing fresh blood and preparing and optionally storing the samples in a manner that prevents degradation of the components of the blood, particularly the RNA and especially the miRNA. Alternatively, an analysis may be performed on stored blood, whether this is stored in the presence of EDTA, Lithium Heparin, in PAXgene Blood RNA tubes or simply (snap-) frozen samples.

The sample extracted from an individual by any means as disclosed above may be analysed essentially immediately, or it may be stored prior to analysis for a variable period of time and at various temperature ranges.

In one embodiment, the sample is stored at a temperature of between −200° C. to 37° C., such as between −200 to −100° C., for example −100 to −50° C., such as −50 to −25° C., for example −25 to −10° C., such as −10 to 0° C., for example 0 to 10° C., such as 10 to 20° C., for example 20 to 30° C., such as 30 to 37° C. prior to analysis. In one embodiment, the sample is stored frozen, such as at −20° C. and/or −80° C.

In another embodiment, the sample is stored for between 15 minutes and 100 years prior to analysis, such as between 15 minutes and 1 hour, for example 1 to 2 hours, such as 2 to 5 hours, for example 5 to 10 hours, such as 10 to 24 hours, for example 24 hours to 48 hours, such as 48 to 72 hours, for example 72 to 96 hours, such as 4 to 7 days, such as 1 week to 2 weeks, such as 2 to 4 weeks, such as 4 weeks to 1 month, such as 1 month to 2 months, for example 2 to 3 months, such as 3 to 4 months, for example 4 to 5 months, such as 5 to 6 months, for example 6 to 7 months, such as 7 to 8 months, for example 8 to 9 months, such as 9 to 10 months, for example 10 to 11 months, such as 11 to 12 months, for example 1 year to 2 years, such as 2 to 3 years, for example 3 to 4 years, such as 4 to 5 years, for example 5 to 6 years, such as 6 to 7 years, for example 7 to 8 years, such as 8 to 9 years, for example 9 to 10 years, such as 10 to 20 years, for example 20 to 30 years, such as 30 to 40 years, for example 40 to 50 years, such as 50 to 75 years, for example 75 to 100 years prior to analysis. In one embodiment, the sample is stored for a few days.

### Collection Media for Sample

A collection media according to the present invention is any media suitable for preserving and/or collecting a sample for immediate or later analysis.

In one embodiment, said collection media is a solution suitable for sample preservation and/or later retrieval of RNA (such as miRNA) from said sample.

In one embodiment, the collection media is an RNA preservation solution or reagent suitable for containing samples without the immediate need for cooling or freezing the sample, while maintaining RNA integrity prior to extraction of RNA (such as miRNA) from the sample. An RNA preservation solution or reagent may also be known as RNA stabilization solution or reagent or RNA recovery media, and may be used interchangeably herein. The RNA preservation solution may penetrate the harvested cells of the collected sample to retard RNA degradation to a rate dependent on the storage temperature.

The RNA preservation solution may be any commercially available solutions or it may be a solution prepared according to available protocols.

The commercially available RNA preservation solutions may for example be selected from RNAlater® (Ambion and Qiagen), PreservCyt medium (Cytyc Corp), PrepProtect™ Stabilisation Buffer (Miltenyi Biotec), Allprotect Tissue Reagent (Qiagen) and RNAprotect Cell Reagent (Qiagen). Protocols for preparing a RNA stabilizing solution may be retrieved from the internet (e.g. L. A. Clarke and M. D. Amaral: ‘Protocol for RNase-retarding solution for cell samples’, provided through The European Working Group on CFTR Expression), or may be produced and/or optimized according to techniques known to the skilled person.

In another embodiment, the collection media will penetrate and lyse the cells of the sample immediately, including reagents and methods for isolating RNA (such as miRNA) from a sample that may or may not include the use of a spin column.

Said reagents and methods for isolating RNA (such as miRNA) is described herein below in the section ‘analysis of sample’.

Other collection media according to the present invention comprises any media such as water, sterile water, denatured water, saline solutions, buffers, PBS, TBS, Allprotect Tissue Reagent (Qiagen), cell culture media such as RPMI-1640, DMEM (Dulbecco's Modified Eagle Medium), MEM (Minimal Essential Medium), IMDM (Iscove's Modified Dulbecco's Medium), BGjB (Fitton-Jackson modification), BME (Basal Medium Eagle), Brinster's BMOC-3 Medium, CMRL Medium, CO₂-Independent Medium, F-10 and F-12 Nutrient Mixture, GMEM (Glasgow Minimum Essential Medium), IMEM (Improved Minimum Essential Medium), Leibovitz's L-15 Medium, McCoy's 5A Medium, MCDB 131 Medium, Medium 199, Opti-MEM, Waymouth's MB 752/1, Williams' Media E, Tyrode's solution, Belyakov's solution, Hanks' solution and other cell culture media known to the skilled person, tissue preservation media such as HypoThermosol®, CryoStor™ and Steinhardt's medium and other tissue preservation media known to the skilled person.

### Sample Analysis

After the sample is collected, it is subjected to analysis. In one embodiment, the sample is initially used for isolating or extracting RNA according to any conventional methods known in the art; followed by an analysis of the miRNA expression in said sample.

### Extraction of RNA

The RNA isolated from the sample may be total RNA, mRNA, microRNA, tRNA, rRNA or any type of RNA.

Conventional methods and reagents for isolating RNA from a sample comprise High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), miRNeasy kit (Qiagen), Oligotex kit (Qiagen), phenol extraction, phenol-chloroform extraction, TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent® (Ambion), RNAqueous Kit (Ambion), ToTALLY RNA™ Kit (Ambion), Poly(A)Purist™ Kit (Ambion) and any other methods, commercially available or not, known to the skilled person.

The RNA may be further amplified, cleaned-up, concentrated, DNase treated, quantified or otherwise analysed or examined such as by agarose gel electrophoresis, absorbance spectrometry or Bioanalyser analysis (Agilent) or subjected to any other post-extraction method known to the skilled person.

Methods for extracting and analysing an RNA sample are disclosed in Molecular Cloning, A Laboratory Manual (Sambrook and Russell (ed.), 3^{rd} edition (2001), Cold Spring Harbor Laboratory Press, Cold Spring Harbor, N.Y., USA.

### Microarray Analysis

The isolated RNA may be analysed by microarray analysis. In one embodiment, the expression level of one or more miRNAs is determined by the microarray technique.

A microarray is a multiplex technology that consists of an arrayed series of thousands of microscopic spots of DNA oligonucleotides or antisense miRNA probes, called features, each containing picomoles of a specific oligonucleotide sequence. This can be a short section of a gene or other DNA or RNA element that are used as probes to hybridize a DNA or RNA sample (called target) under high-stringency conditions. Probe-target hybridization is usually detected and quantified by fluorescence-based detection of fluorophore-labeled targets to determine relative abundance of nucleic acid sequences in the target. In standard microarrays, the probes are attached to a solid surface by a covalent bond to a chemical matrix (via epoxy-silane, amino-silane, lysine, polyacrylamide or others). The solid surface can be glass or a silicon chip, in which case they are commonly known as gene chip. DNA arrays are so named because they either measure DNA or use DNA as part of its detection system. The DNA probe may however be a modified DNA structure such as LNA (locked nucleic acid).

In one embodiment, the microarray analysis is used to detect microRNA, known as microRNA or miRNA expression profiling.

The microarray for detection of microRNA may be a microarray platform, wherein the probes of the microarray may be comprised of antisense miRNAs or DNA oligonucleotides. In the first case, the target is a labelled sense miRNA sequence, and in the latter case the miRNA has been reverse transcribed into cDNA and labelled.

The microarray for detection of microRNA may be a commercially available array platform, such as NCode™ miRNA Microarray Expression Profiling (Invitrogen), miRCURY LNA™ microRNA Arrays (Exiqon), microRNA Array (Agilent), μParaflo® Microfluidic Biochip Technology (LC Sciences), MicroRNA Profiling Panels (Illumina),

Geniom® Biochips (Febit Inc.), microRNA Array (Oxford Gene Technology), Custom AdmiRNA™ profiling service (Applied Biological Materials Inc.), microRNA Array (Dharmacon—Thermo Scientific), LDA TaqMan analyses (Applied Biosystems), Taqman microRNA Array (Applied Biosystems), Biomark™ HD System (Fluidigm System) using TaqMan reagents or any other commercially available array.

Microarray analysis may comprise all or a subset of the steps of RNA isolation, RNA amplification, reverse transcription, target labelling, hybridisation onto a microarray chip, image analysis and normalisation, and subsequent data analysis; each of these steps may be performed according to a manufacturers protocol. In another embodiment, the microarray for detection of microRNA is custom made.

It follows, that any of the methods as disclosed herein above e.g. for diagnosing of an individual may further comprise one or more of the steps of:
i) isolating miRNA from a sample,
ii) labelling of said miRNA,
iii) hybridising said labelled miRNA to a microarray or QPCR assay comprising miRNA-specific probes to provide a hybridisation profile for the sample,
iv) performing data analysis to obtain a measure of the miRNA expression profile of said sample.

A probe or hybridization probe is a fragment of DNA or RNA of variable length, which is used to detect in DNA or RNA samples the presence of nucleotide sequences (the target) that are complementary to the sequence in the probe. One example is a sense miRNA sequence in a sample (target) and an antisense miRNA probe. The probe thereby hybridizes to single-stranded nucleic acid (DNA or RNA) whose base sequence allows probe-target base pairing due to complementarity between the probe and target.

To detect hybridization of the probe to its target sequence, the probe or the sample is tagged (or labeled) with a molecular marker. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in microarrays refer to nucleotide sequences covalently attached to an inert surface, such as coated glass slides, and to which a mobile target is hybridized. Depending on the method the probe may be synthesised via phosphoramidite technology or generated by PCR amplification or cloning (older methods). To design probe sequences, a probe design algorithm may be used to ensure maximum specificity (discerning closely related targets), sensitivity (maximum hybridisation intensities) and normalised melting temperatures for uniform hybridisation.

### RT-QPCR

The isolated RNA may be analysed by quantitative (‘real-time’) PCR (QPCR). In one embodiment, the expression level of one or more miRNAs is determined by the quantitative polymerase chain reaction (QPCR) technique.

Real-time polymerase chain reaction, also called quantitative polymerase chain reaction (Q-PCR/qPCR/RT-QPCR) or kinetic polymerase chain reaction, is a technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA. Frequently, real-time polymerase chain reaction is combined with reverse transcription polymerase chain reaction to quantify low abundance messenger RNA (mRNA), or miRNA, enabling a researcher to quantify relative gene expression at a particular time, or in a particular cell or tissue type.

In a real time PCR assay a positive reaction is detected by accumulation of a fluorescent signal. The Ct (cycle threshold) is defined as the number of cycles required for the fluorescent signal to cross the threshold (i.e. exceeds background level). Ct-values are inversely proportional to the amount of target nucleic acid in the sample (i.e. the lower the Ct-value the greater the amount of target nucleic acid in the sample). Most real time assays undergo 40 cycles of amplification.

Ct-values <29 are strong positive reactions indicative of abundant target nucleic acid in the sample. Ct-values of 30-37 are positive reactions indicative of moderate amounts of target nucleic acid. Ct-values of 38-40 are weak reactions indicative of minimal amounts of target nucleic acid which could represent an infection state or environmental contamination.

The QPCR may be performed using chemicals and/or machines from a commercially available platform.

The QPCR may be performed using QPCR machines from any commercially available platform; such as Prism, geneAmp or StepOne Real Time PCR systems (Applied Biosystems), LightCycler (Roche), RapidCycler (Idaho Technology), MasterCycler (Eppendorf), BioMark™ HD System (Fluidigm), iCycler iQ system, Chromo 4 system, CFX, MiniOpticon and Opticon systems (Bio-Rad), SmartCycler system (Cepheid), RotorGene system (Corbett Lifescience), MX3000 and MX3005 systems (Stratagene), DNA Engine Opticon system (Qiagen), Quantica qPCR systems (Techne), InSyte and Syncrom cycler system (BioGene), DT-322 (DNA Technology), Exicycler Notebook Thermal cycler, TL998 System (lanlong), Line-Gene-K systems (Bioer Technology), or any other commercially available platform.

The QPCR may be performed using chemicals from any commercially available platform, such as NCode EXPRESS qPCR or EXPRESS qPCR (Invitrogen), Taqman or SYBR green qPCR systems (Applied Biosystems), Real-Time PCR reagents (Eurogentec), iTaq mix (Bio-Rad), qPCR mixes and kits (Biosense), and any other chemicals, commercially available or not, known to the skilled person.

The QPCR reagents and detection system may be probe-based, or may be based on chelating a fluorescent chemical into double-stranded oligonucleotides.

The QPCR reaction may be performed in a tube; such as a single tube, a tube strip or a plate, or it may be performed in a microfluidic card in which the relevant probes and/or primers are already integrated.

A Microfluidic card allows high throughput, parallel analysis of mRNA or miRNA expression patterns, and allows for a quick and cost-effective investigation of biological pathways. The microfluidic card may be a piece of plastic that is riddled with micro channels and chambers filled with the probes needed to translate a sample into a diagnosis. A sample in fluid form is injected into one end of the card, and capillary action causes the fluid sample to be distributed into the microchannels. The microfluidic card is then placed in an appropriate device for processing the card and reading the signal.

### Other Analysis Methods

The isolated RNA may be analysed by northern blotting. In one embodiment, the expression level of one or more miRNAs is determined by the northern blot technique.

A northern blot is a method used to check for the presence of a RNA sequence in a sample. Northern blotting combines denaturing agarose gel or polyacrylamide gel electrophoresis for size separation of RNA with methods to transfer the size-separated RNA to a filter membrane for probe hybridization. The hybridization probe may be made from DNA or RNA.

In yet another embodiment, the isolated RNA is analysed by nuclease protection assay. The isolated RNA may be analysed by Nuclease protection assay.

Nuclease protection assay is a technique used to identify individual RNA molecules in a heterogeneous RNA sample extracted from cells. The technique can identify one or more RNA molecules of known sequence even at low total concentration. The extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest.

### Device

It is also an aspect of the present invention to provide a device for measuring the expression level of at least one miRNA in a sample, wherein said device comprises or consists of at least one probe or probe set for one or more miRNA selected from the group consisting of:
a. let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941,
b. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645
   wherein said device is used for characterising a sample.

In one embodiment, the device may be used for distinguishing between patients with pancreas cancer (PAC and/or AAC) and individuals with normal pancreas and/or chronic pancreatitis.

In one embodiment, said device may be used with the miRNA classifier according to the present invention to classify a sample as pancreatic carcinoma, normal pancreas or chronic pancreatitis.

In one embodiment, the device may be used in a method for estimating the probability for a patient with pancreatic cancer of surviving for a certain time period, said method comprising measuring the expression level of at least one miRNA in a sample obtained from said individual.

In one embodiment, the device may be a microarray chip; a QPCR Micro Fluidic Card; or may comprise QPCR tubes, QPCR tubes in a strip or a QPCR plate, comprising one or more probes for at least one miRNA and identified herein. The probes may be comprised on a solid support, on at least one bead, or in a liquid reagent comprised in a tube.

### Computer Program Product

It is a further aspect of the invention to provide a computer program product having a computer readable medium, said computer program product comprising means for carrying out any of the herein listed miRNA classifiers, models and methods.

It is a further aspect of the invention to provide a system comprising means for carrying out any of the herein listed methods.

In another aspect, the present invention provides a computer program product having a computer readable medium, said computer program product providing a system for predicting the diagnosis of an individual, said computer program product comprising means for carrying out any of the steps of any of the methods as disclosed herein.

In another aspect, the present invention provides a system as disclosed herein wherein the data is stored, such as stored in at least one database.

### Kit-of-Parts

It is also an aspect to provide a kit-of-parts comprising the device according to the present invention, and at least one additional component.

In one embodiment, the additional component may be used simultaneously, sequentially or separately with the device.

In one embodiment, said additional component comprises means for extracting RNA such as miRNA from a sample; reagents for performing microarray analysis and/or reagents for performing QPCR analysis.

In another embodiment, said kit may comprise instructions for use of the device and/or the additional components.

In a further embodiment, said kit comprises a computer program product having a computer readable medium as detailed herein elsewhere.

Mature sequence of miRNAs
from multivariate analyses

| miRBase name¹ | Mature sequence² |
|---|---|
| hsa-miR-1 | UGGAAUGUAAAGAAGUAUGUAU |
| hsa-miR-19b | UGUGCAAAUCCAUGCAAAACUGA |
| hsa-miR-20a | UAAAGUGCUUAUAGUGCAGGUAG |
| hsa-miR-23b | AUCACAUUGCCAGGGAUUACC |
| hsa-miR-26a | UUCAAGUAAUCCAGGAUAGGCU |
| hsa-miR-26b | UUCAAGUAAUUCAGGAUAGGU |
| hsa-miR-27a | UUCACAGUGGCUAAGUUCCGC |
| hsa-miR-29c | UAGCACCAUUUGAAAUCGGUUA |
| hsa-miR-30a-5p | UGUAAACAUCCUCGACUGGAAG |
| hsa-miR-30b | UGUAAACAUCCUACACUCAGCU |
| hsa-miR-30e-3p | CUUUCAGUCGGAUGUUUACAGC |
| hsa-miR-30c | UGUAAACAUCCUACACUCUCAGC |
| hsa-miR-31 | AGGCAAGAUGCUGGCAUAGCU |
| hsa-miR-99b | CACCCGUAGAACCGACCUUGCG |
| hsa-miR-100 | AACCCGUAGAUCCGAACUUGUG |
| hsa-miR-106a | AAAAGUGCUUACAGUGCAGGUAG |
| hsa-miR-150 | UCUCCCAACCCUUGUACCAGUG |
| hsa-miR-185 | UGGAGAGAAAGGCAGUUCCUGA |
| hsa-miR-190 | UGAUAUGUUUGAUAUAUUAGGU |
| hsa-miR-195 | UAGCAGCACAGAAAUAUUGGC |
| hsa-miR-196b | UAGGUAGUUUCCUGUUGUUGGG |
| hsa-miR-212 | UAACAGUCUCCAGUCACGGCC |
| hsa-miR-296-3p | GAGGGUUGGGUGGAGGCUCUCC |
| hsa-miR-320 | AAAAGCUGGGUUGAGAGGGCGA |
| hsa-miR-323-3p | CACAUUACACGGUCGACCUCU |
| hsa-miR-324-3p | ACUGCCCCAGGUGCUGCUGG |
| hsa-miR-326 | CCUCUGGGCCCUUCCUCCAG |
| hsa-miR-331-3p | GCCCCUGGGCCUAUCCUAGAA |
| hsa-miR-345 | GCUGACUCCUAGUCCAGGGCUC |
| hsa-miR-362-3p | AACACACCUAUUCAAGGAUUCA |
| hsa-miR-370 | GCCUGCUGGGGUGGAACCUGGU |
| hsa-miR-450a | UUUUGCGAUGUGUUCCUAAUAU |
| hsa-miR-450b-5p | UUUUGCAAUAUGUUCCUGAAUA |
| hsa-miR-451 | AAACCGUUACCAUUACUGAGUU |
| hsa-miR-483-5p | AAGACGGGAGGAAAGAAGGGAG |
| hsa-miR-484 | UCAGGCUCAGUCCCUCCCGAU |
| hsa-miR-485-3p | GUCAUACACGGCUUCCUUCU |
| hsa-miR-511 | GUGUCUUUUGCUCUGCAGUCA |
| hsa-miR-574-3p | CACGCUCAGCACACACCCACA |
| hsa-miR-590-5p | GAGCUUAUCAUAAAAGUGCAG |
| hsa-miR-618 | AAACUCUAUUGUCCUUCUGAGU |
| hsa-miR-636 | UGUGCUUGCUCGUCCCGCCCGCA |
| hsa-miR-638 | AGGGAUCGCGGGCGGGUGGCGGCCU |
| hsa-miR-645 | UCUAGGCUGGUACUGCUGA |
| hsa-miR-758 | UUUGUGACUGGUCCAUAACC |
| hsa-miR-875-3p | CCUGGAAACACUGAGGUUGUG |
| hsa-miR-874 | CUGCCCUGGCCCGAGGAA |
| hsa-miR-1238 | CUUCCUCGUCUGUCUGCCCC |

¹miRBase 18, November 2011, www.mirbase.org (homo sapiens), accessed Jan. 14^{th}, 2012
²www.appliedbiosystems.com, accessed Jan. 14^{th}, 2012

### EXAMPLES

### Example 1

### MicroRNA Expression Profiles in Blood as Biomarkers for Early Diagnosis and Prognosis of Patients with Pancreatic Cancer

The aim of the present study was to identify new diagnostic miRNAs in serum, plasma and whole blood from patients with pancreatic cancer (PC). The study was conducted according to the REMARK guidelines (23).

### Patients

### BIOPAC Study:

470 patients were included between Jul. 1, 2008 and Dec. 31, 2011 in the multicenter prospective biomarker study “BIOmarkers in patients with PAncreatic Cancer (BIOPAC)—can they provide new information of the disease and improve diagnosis and prognosis of the patients?”. Blood samples were collected at time of diagnosis and before and during treatment. The patients with localized PC were treated with operation followed by adjuvant gemcitabine. The patients with locally advanced or metastatic PC were treated with palliative gemcitabine. Patients were followed from their date of inclusion and until death, or censoring Jan. 2, 2012, whichever came first. All patients provided written informed consent and the study was approved by the Regional Ethics Committee (VEK ref. KA-20060113).

### Heidelberg Study:

131 patients were included in the pancreas cancer biomarker study at Heidelberg University. Blood samples were collected at time of diagnosis and before treatment. All patients had localized PC and were treated with operation followed by adjuvant gemcitabine. Patients were followed from their date of operation and until death or censoring whichever came first. All patients provided written informed consent, and the study was approved by the Regional Ethics Committee.

### Controls

300 healthy blood donors and 40 patients with chronic pancreatitis (CP).

### Methods

Purification of miRNA in Serum and Plasma:

Blood samples for serum and plasma analysis were centrifuged at 2300g within 2 hours after blood sampling and stored at −80° C. until analysis. MiRNA in serum and plasma samples were purified according to the miRNeasy mini kit protocol from Qiagen (Cat no. 217004).

Purification of miRNA in Whole Blood:

Blood samples were collected in PAXgene Blood RNA tubes (Qiagen) and treated according to the manufacturer's instructions. Small RNAs were extracted from the PAXgene Blood RNA tubes in two fractions (24). The PAXgene Blood RNA tubes were processed on the Biorobot MDx (Qiagen, Hilden, Germany) using a customized protocol that binds large RNAs and rescues the run-through from the RNA binding plate. The binding condition in the run-through was subsequently modified enabling the miRNA to be purified on an RNeasy-96 plate. The concentration of the small RNA fractions was assessed by absorbance spectrometry on a DTX 880 (Beckman Coulter).

### MiRNA Expression Analysis in “Pilot Study I” and the “Discovery Study”:

The TaqMan® Human MicroRNA assay using A Cards v2.0 and B Cards Set v3.0 (Part Number 4400238, Applied Biosystem) was used. This method used a set of two pre-configured micro fluidic cards that enables quantization of 754 human miRs. Included on each array were three TaqMan MicroRNA assay endogenous controls to aid in data normalization and one TaqMan® MicroRNA assay not related to human as a negative control. RNA was transcribed into cDNA in two multiplex reactions each containing 3 μl of the small RNA preparation and either Megaplex RT Primer A Pool or Pool B pool and using the TaqMan MicroRNA Reverse Transcription Kit in a total volume of 14 μl. Prior to loading of the arrays a 12 cycle preamplification reaction was performed using 2.5 μl cDNA in a 25 μl reaction. Each of the arrays was loaded with 800 μl Universal PCR MasterMix assay containing 1/40 of the preamplification reaction and run on the 7900HT Fast Real-Time PCR System. The instruction from Applied Biosystems was followed in all details including the use of pre-amplification (https://www.products.appliedbiosystems.com). This analysis was performed at the biotech company AROS, Applied Biotechnology A/S, Denmark.

### MiRNA Expression Analysis in “Pilot Study II”:

The LNA technology (miRCURY LNA™ Universal RT microRNA PCR, Exiqon). This technique allows that all miRNAs can be measured from a single reverse transcription reaction (this converts the extracted RNA to DNA that then can be measured with PCR). A total of 640 human miRNAs were determined. The instructions from Exiqon were followed in all details (https://www.exiqon.com). This analysis was performed at the biotech company Exiqon, Denmark.

### MiRNA Expression Analysis in the “Validation Studies”:

We will analyze 46 different miRNAs (selected from the “Discovery Studies”) using the Fluidigm BioMark™ System. This array system can perform 2,304 simultaneous real-time PCR experiments running gold-standard TaqMan® assays in nanolitre quantities. The instruction from Fluidigm will be followed in all details (https://www.fluidigm.com). This analysis will be performed at the biotech company AROS, Applied Biotechnology A/S, Denmark.

### Statistical Analysis

Data was normalized to adjust for block effects (between-array variation). Raw Ct-values where pre-processed using either: 1) rank normalization; 2) quantile normalization (25,26); or 3) normalization using endogene controls. Rank normalization is done for each patient to ranking the Ct-values for the miRNAs such that the lowest value gets rank 1 and so on. Normalization by endogene controls was done by subtracting the mean value of the endogene controls (Mamm and U6) for each patient from the Ct-values. Normalized data was inspected for outliers and potential technical bias from sample quality, sample purification date and array batch (27,28). There are two different outcomes under study: Diagnosis (pancreas cancer or not) and overall survival. For analysis on diagnosis logistic regression models are used (29-31), whereas for survival outcome Cox proportional hazards model is used (32,33). The univariate selection method implies estimating and testing each miRNA expression value on survival (or diagnosis) univariately. This was done by fitting the Cox proportional hazard (or the logistic regression) model and testing each miRNA separately. All miRNAs that met the 0.001 significance level for serum and 0.01 for whole blood in the univariate analysis were then kept and included in a multivariate Cox proportional hazard (or logistic regression) model. The final model was obtained by backwards elimination of the multivariate model using Akaike's Information Criterion (AIC) (34). For the Cox proportional hazards model the estimates were adjusted for age and gender.

The statistical software R (35) version 2.14.0 was used in all analysis. We used the package survival version 2.36-10 for fitting the Cox proportional hazard model and the library stats version 2.14.0 for the logistic regression model.

### Results

Association of miRNAs with Pancreatic Cancer (Serum, Plasma and Whole Blood)

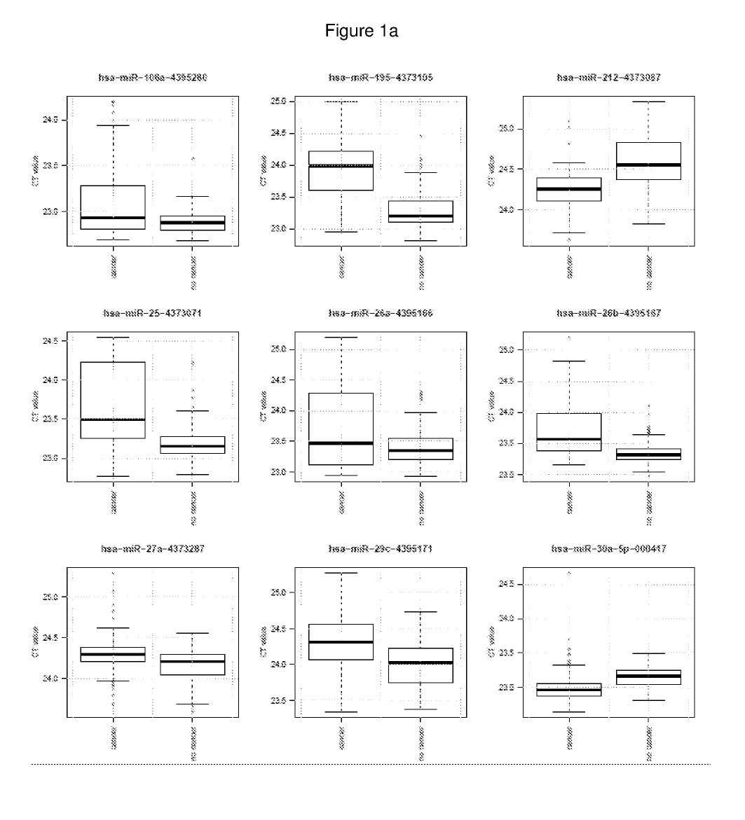

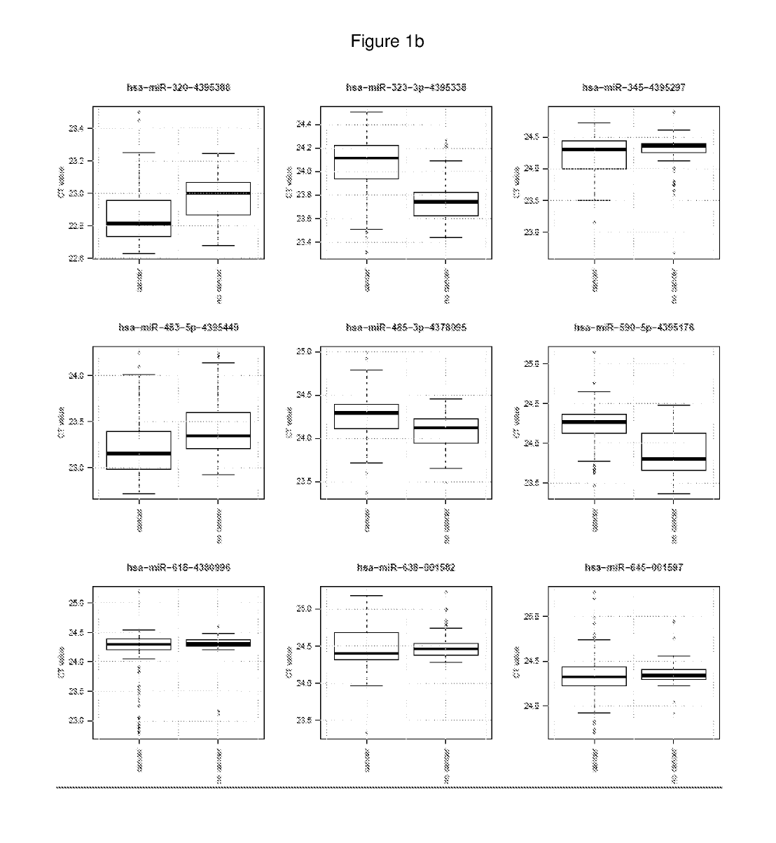

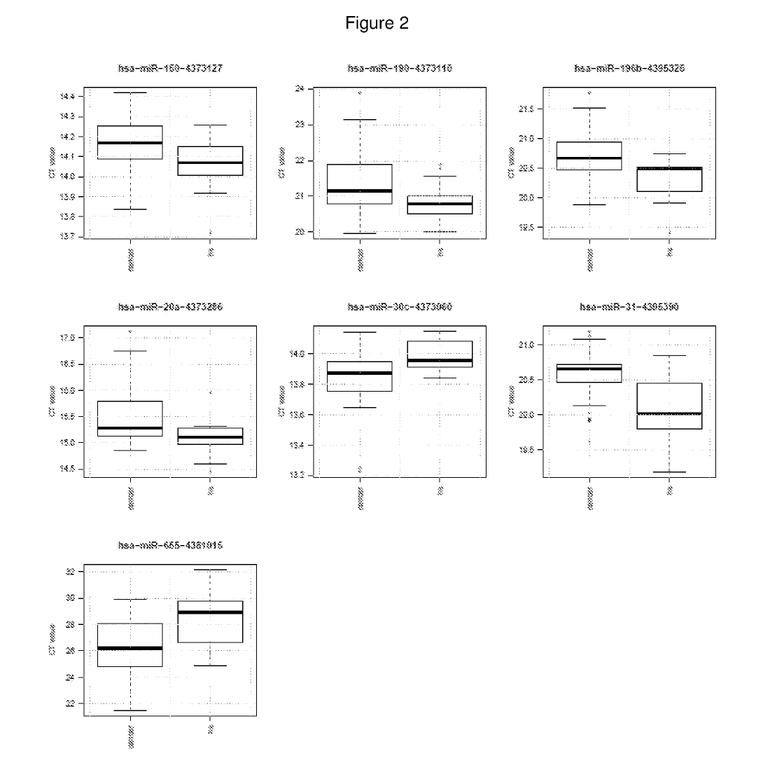

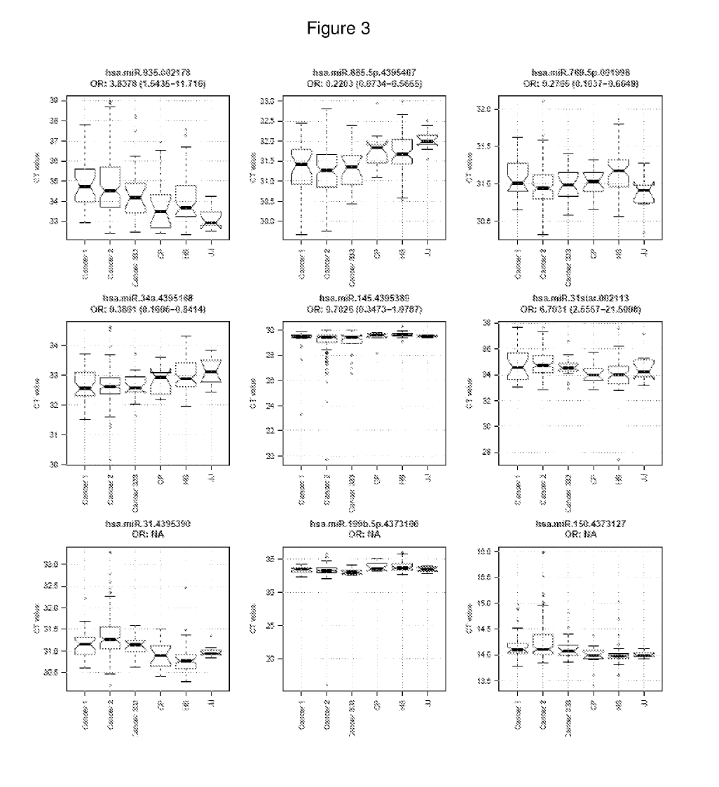

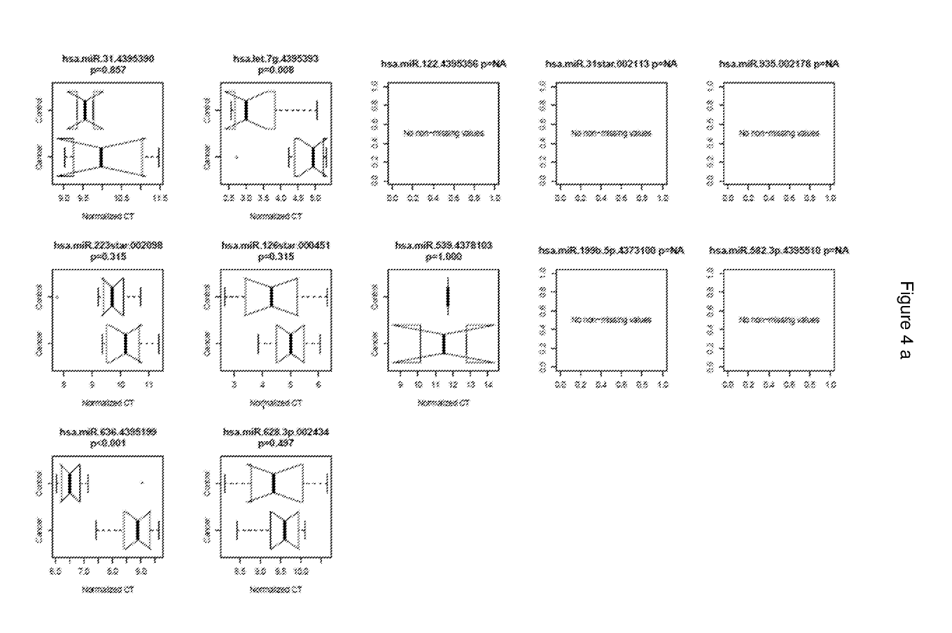

We used two different methods for miRNA determination in the Pilot studies. FIG. 1 illustrates the miRNAs results using the TaqMan® Human MicroRNA assay from Applied Biosystem for determination of miRNAs in serum (1A), EDTA plasma (1B) and whole blood (1C) from “Pilot Study I” of 10 patients with PC and 10 healthy subjects. FIG. 2 illustrates the results using the miRCURY LNA™ Universal RT microRNA PCR assay from Exiqon for determination of miRNAs in serum from “Pilot Study II” of 25 patients with PC and 25 healthy subjects demonstrating. Using this method we found that 26 miRNAs in serum were highly significantly differently expressed between the two groups.

Table 1 shows the miRNAs found in the Pilot studies which were significantly differently expressed between patients with pancreatic cancer (PC) and controls (ie. healthy subjects and patients with chronic pancreatitis (CP)).

Table 2 shows miRNAs in serum that can separate patients with pancreatic cancer (PC) (n=139) from healthy subjects (n=50) and patients with chronic pancreatitis (CP) (n=17). Three different types of normalization were used. Using this method the following eighteen miRs were significantly associated with pancreatic cancer (PC): miR-25, miR-26a, miR-26b, miR-27a, miR-29c, miR-30a-5p, miR-106a, miR-195, miR-212, miR-320, miR-323-3p, miR-345, miR-483-5p, miR-485-3p, miR-590-5p, miR-618, miR-638, and miR-645. For these miRNAs either decreased expression (high Ct-value) (miRNAs: miR-25, miR-26a, miR-26b, miR-27a, miR-30a-5p, miR-195, miR-323-3p, miR-345, miR-485-3p, miR-618, and miR-645) or increased expression (low Ct-value) (miRNAs: miR-29c, miR-106a, miR-212, miR-320, miR-483-5p, miR-590-5p, and miR-638) were associated with pancreatic cancer (PC).

Table 3 shows miRNAs in whole blood that can separate patients with pancreatic cancer (PC) (n=41) from healthy subjects (n=17) and patients with chronic pancreatitis (CP) (n=4). Three different types of normalization were used. Using this method the following six miRs were significantly associated with pancreatic cancer (PC): miR-20a, miR-30c, miR-31, miR-150, miR-190, and miR-196b. For these miRNAs either decreased expression (high Ct-value) (miRNAs: miR-20a, miR-31, miR-150, miR-190, mir-196b) or increased expression (low Ct-value) (miRNA: miR-30c) were associated with pancreatic cancer (PC).

Association of miRNAs with Overall Survival

In the survival analysis of 104 patients with pancreatic cancer (PC) and using the Cox proportional hazard model with backwards elimination and using either rank and quantile normalization the following miRNAs in serum were prognostic for overall survival (OS) (Table 4): miR-19b, miR-27a, miR-30b, miR-30e-3p, miR-99b, miR-100, miR-181a, miR-185, miR-331-3p, miR-511, miR-362-3p, miR-758 and miR-1238. The hazard ratios, calculated per unit increase for the 40 minus Ct-value of these miRNAs, are shown in Table 4 for serum (and in Table 5 for whole blood). As seen in Table 4: Either low expression (high Ct-value) (miRNAs: miR-27a, mir-30b, miR-100, miR-181a, miR-185, miR-331-3p, miR-511) or high expression (low Ct-value) (miRNAs: miR-30e-3p, miR-362-3p, and miR-758) predicted short overall survival (OS) independent of age and gender. Low expression (high Ct-value) of miR-19b and miR-99b predicted overall survival (OS) in the unadjusted analysis.

In the survival analysis of 41 patients with pancreatic cancer (PC) and using the Cox proportional hazard model with backwards elimination and using either rank and quantile normalization the following miRNAs in whole blood were prognostic for overall survival (OS) (Table 5): miR-1, miR-23b, miR-27a, miR-150, miR-296-3p, miR-324-3p, miR-326, miR-370, miR-450a, miR-450b-5p, miR-451, miR-484, miR-574-3p, miR-636, miR-874, and miR-875-3p. The hazard ratios, calculated per unit increase for the 40 minus Ct-value of these miRNAs, are shown in Table 5 for whole blood. As seen in Table 5: Either low expression (high Ct-value) (miRNAs: miR-1, miR-150, miR-324-3p, miR-326, miR-370, miR-874, miR-875-3p) or high expression (low Ct-value) (miRNAs: miR-27a, miR-296-3p, miR-450a, miR-450b-5p, miR-451, and miR-574-3p) predicted short overall survival (OS) independent of age and gender. Low expression (high Ct-value) of miR-484 and high expression of (low Ct-value) of miR-23b and miR-636 predicted overall survival (OS) in the unadjusted analysis.

Association of Diagnostic and Prognostic miRNAs Described in the Literature Related to Pancreatic Cancer

In our studies we could confirm the results from the literature of the following diagnostic and prognostic miRNAs in serum and plasma: let-7 family, miR-16, miR-18a, miR-20a, miR-21, mir-24, miR-25, miR-99, miR-146, miR-155, miR-181a, miR-181b, miR-185, miR-191, miR-196a, and miR-210. From the scientific literature no studies were found describing miRNA expression in whole blood from patients with pancreatic cancer (PC).

### Discussion

Less than 20% of patients with pancreatic cancer (PC) can today be operated with curative intentions, since the disease is most often diagnosed at advanced stage. Even patients who are operated have at poor prognosis with only 23% alive after 5-years (2). Better biomarkers to identify patients with pancreatic cancer (PC) at an early stage and identification of those with the poorest prognosis are needed in order to treat these patients more aggressively. Our studies include novel observations by identification of several new miRNAs for diagnosis of pancreatic cancer (PC) and by combining miRNAs in both serum and whole blood; this is a new way to identify patients with pancreatic cancer (PC) and to predict which patients have the poorest prognosis.

It is therefore a strength of our studies that we both analyzed miRNAs expression profiles in serum, plasma and whole blood, the large number of patients and controls, the large number of miRNAs analyzed and that we used three different array platforms (TaqMan® Human MicroRNA assay from Applied Biosystem, TaqMan® using the Biomark™ HD System platform from Fluidigm, and miRCURY LNA™ Universal RT microRNA PCR assay from Exiqon) and several different statistical methods for normalization. The identified miRNAs are therefore likely to be validated as useful diagnostic biomarkers in future large studies of patients with pancreatic cancer, chronic pancreatitis and healthy subjects and finally to be useful for daily clinical practice of early diagnosis of patients with pancreatic cancer (PC) and to a better prediction of prognosis.

### Example 2

### Analysis of microRNAs Purified from Whole Blood Collected in PAXgene RNA Tubes and Analyzed Using the LDA-Card Platform

In this example, the ‘final dataset’ is defined for the discovery phase for diagnosis of pancreatic cancer and miRNAs purified from whole-blood collected in PAXgene RNA tubes and analyzed on LDA-cards (TaqMan LDA microfluidic card technology from Applied Biosystems; Foster City, Calif., USA).

### Final Dataset (Including Removal of Outliers)

The data consists of 280 samples on which more than 700 miRs have been measured. The experiment was designed such that age, sex and diagnosis were balanced out on day of miR purification and furthermore age, sex, diagnosis and day of purification were balanced out on day of miR analysis.

The final data set consists of 276 samples. Four samples have been excluded to the following reasons
1 sample has an absorbance (260/280) below 1.80
2 samples have a high mean CT and also many missing values
1 sample has many missing values (but the mean CT is normal)

### Data Analysis

The analysis of the incidence is based on the 276 samples, which are left after removing outliers. 21 cancer cases had their blood samples taken after operation and thus they are left out of the analysis in the first place. Moreover, 10 samples from JJ are also omitted from the analysis and instead used together with the 21 cancers for validation.

The data is normalized using 5 different normalizations:
Endogene controls based on the RNU-family.
Quantile normalization.
Rank normalization
Mean of the 120 most expressed miRs normalization
Raw values

For all normalization techniques the endogene controls are removed before normalization (except for the endogene control normalization where the mean of the endogene controls is used). The number of missing values allowed for a miR was set to 10% correspond to 27 (for the rank method this does not matter since missing values are assigned a rank).

We model the incidence of cancer, i.e., the log-odds. The estimates are presented as OR for an interquartile range increase in the CT-value (or rank for the rank normalization). This implies that OR>1 means that the incidence increases for increasing CT-value, which corresponds to increasing incidence for decreasing expression (i.e. OR>1=high CT-value=low expression in cancer patient samples).

For all normalizations univariate p-values are computed and based on these a set of miRs is included in a multivarite model which is reduced by a back-wards elimination procedure. The cut-off for inclusion in the multivariate model was set to 0.001 with the restriction that no more than 40 miRs were included. The significant miRs for each normalization were compiled to a single file in which ORs, confidence intervals, p-values and the number of normalizations having this miR were reported for each potential miR. Moreover the univariate p-values were recorded and subsequently compared. Finally the probability of cancer was predicted using the models corresponding to the 5 normalizations.

Significant miRs

The miRs selected by either of the 5 normalization methods are given in Table 6. The last column indicates by how many normalization methods the miR is selected and the table is ordered after this column. It can be seen that the maximum number of times a miR is selected is four, which is the case for hsa.miR.935.002178 (=hsa-miR-935), hsa.miR.885.5p.4395407 (=hsa-miR-885.3p), hsa.miR.769.5p.001998 (=hsa-miR-769.5p), hsa.miR.34a.4395168 (=hsa-miR-34a) and hsa.miR.145.4395389 (=hsa-miR-145). It can be seen that for these five miRs the method with the endogene controls is missing the miR in four out of 5. Another observation is that mir-31 (in either the starred or non-starred version) is present for all normalization methods and all with OR>1. Using the endogene controls mir31 is present in the model with both the starred and non-starred version. mir-223 is found with two normalization methods: 120 most expressed (non-starred) and endogene controls (starred). Likewise the let 7 family (g and b) is found by raw values and endogene control normalization.

For the identified miRNAs either decreased expression (OR>1; high Ct-value) (miR-150, let-7b, let-7g, miR-9*, miR-19b, miR-23a, miR-24.2*, miR-31, miR-31*, miR-93, miR-143, miR-144*, miR-342.5p, miR-345, miR-362.3p, miR-374b*, miR-508.3p, miR-539, miR-628.3p, miR-636, miR-935 and miR-636/quantile normalization) or increased expression (OR<1; low Ct-value) (miR-30c, miR-26b, miR-30b, miR-34a, miR-122, miR-126*, miR-128, miR-145, miR-186, miR-199b.5p, miR-223, miR-223*, miR-505, miR-625, miR-636, miR-769.5p, miR-885.5p, miR-941 and miR-636/endogene normalization) were associated with pancreatic cancer (PC)—see Table 6.

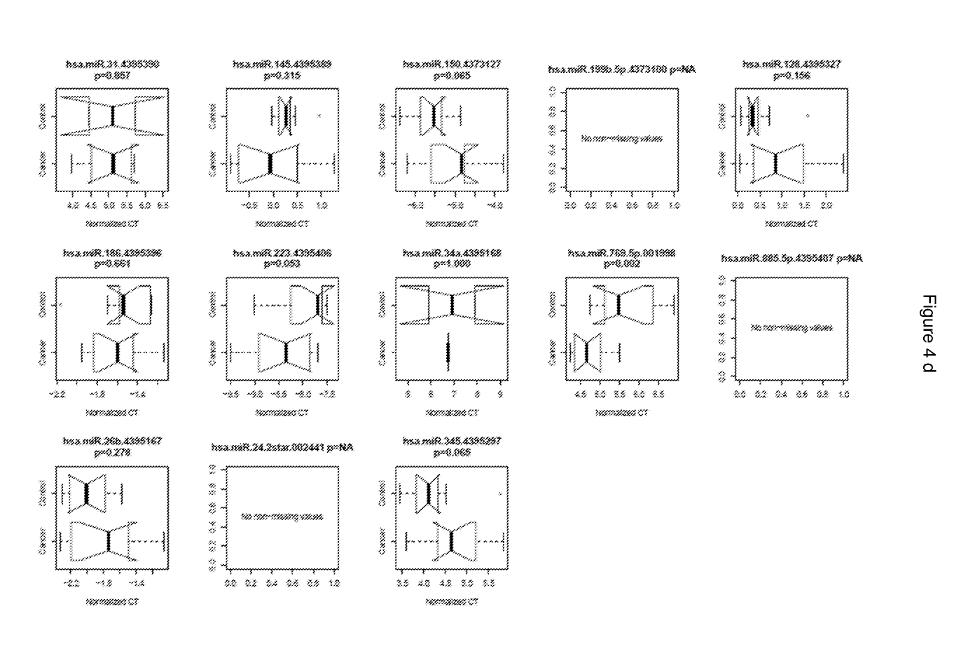

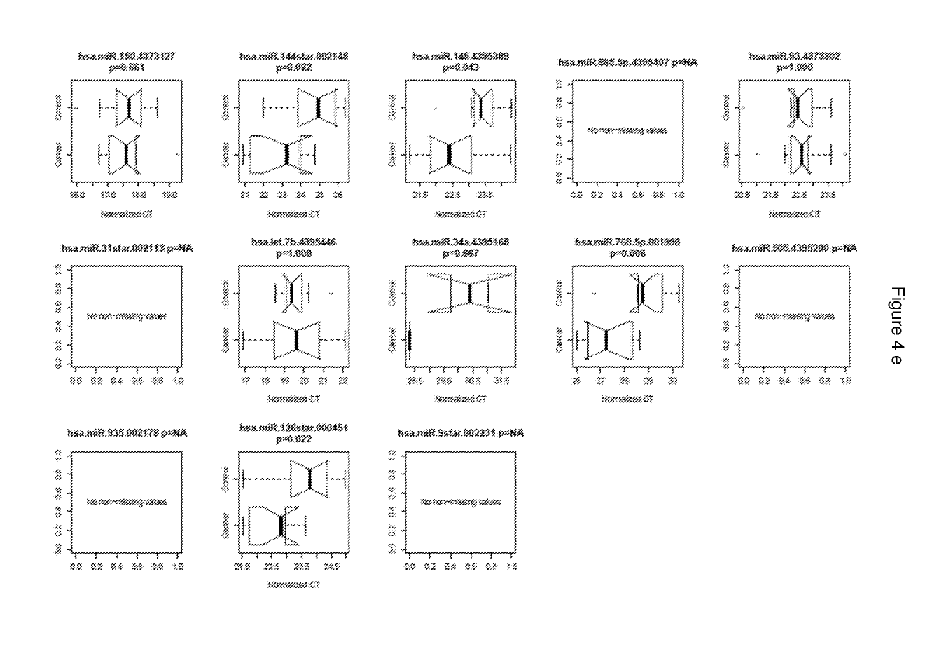

Furthermore boxplots of the first 9 miRs from Table 6 are given in FIG. 5, which corresponds to miRs that are chosen by at least 3 different normalization techniques. It can also be seen that the samples from cancer patients taken after operation appear to be similar to the other cancer samples.

### Analysis of the Incidence of Cancer

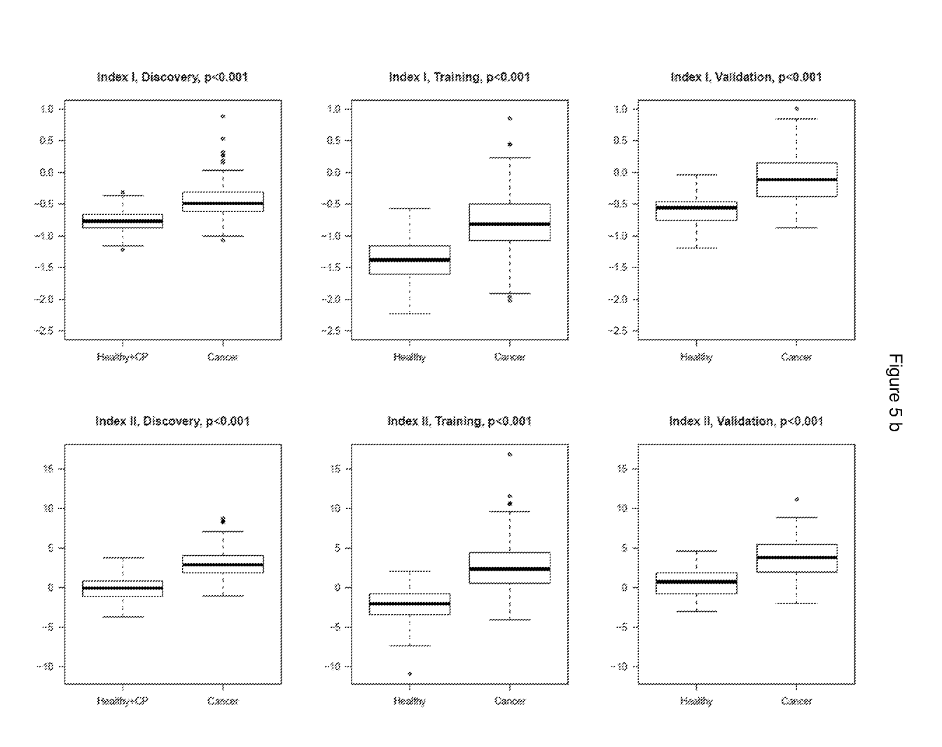

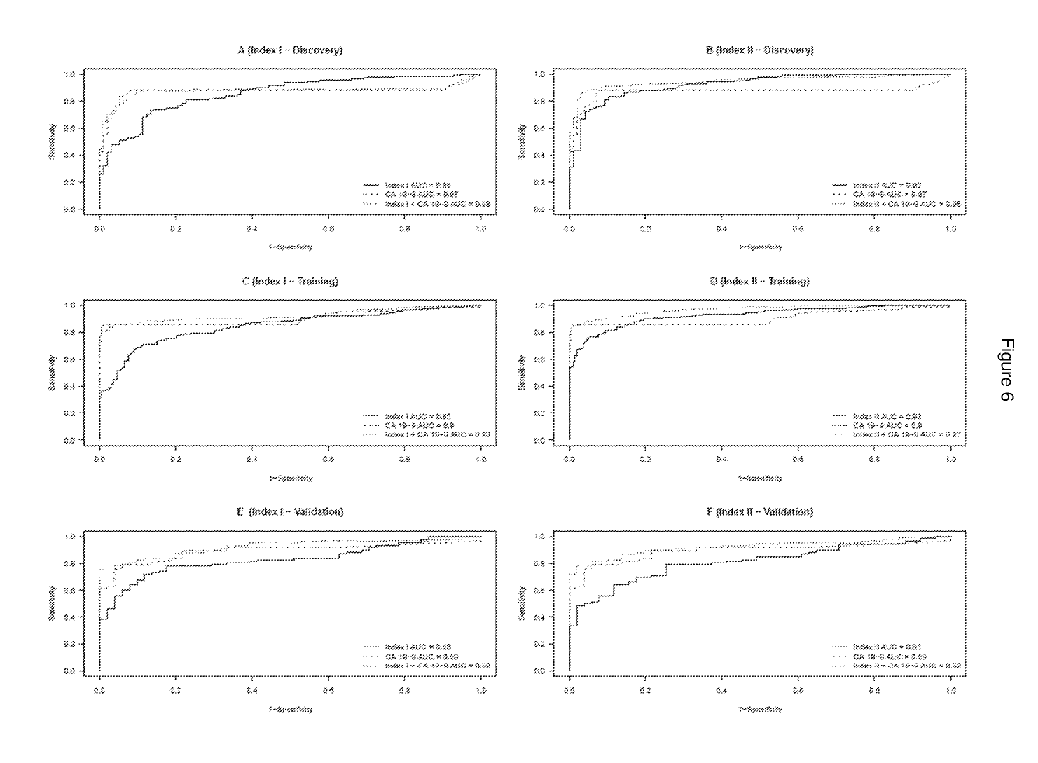

The predicted probability of cancer for the cancer samples taken after operation is plotted in FIG. 7. It can be seen that they mostly are classified correctly as cancers. Only 1 to 4 samples depending on the normalization method have a probability less than 50% of being cancer (the actual cut-off point could potentially be somewhere else). The predicted probabilities are given in Table 7, which shows that patient number 01-100 is the only sample consistently getting a low probability. Three other candidates are 01-077, 01-211 and 01-219. Thus, the predicted probability of cancer is correct in 96 (value above 50%) of total 107 predictions made (22 patients×5 normalization methods, minus 3 missing values); i.e. 89.7%.

### Pilot Study Samples

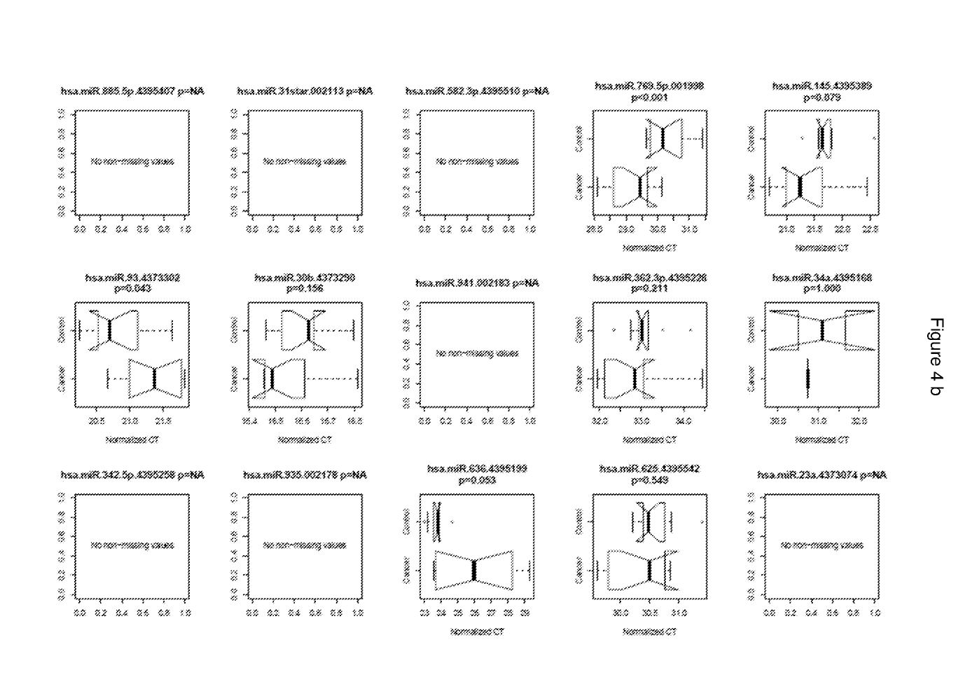

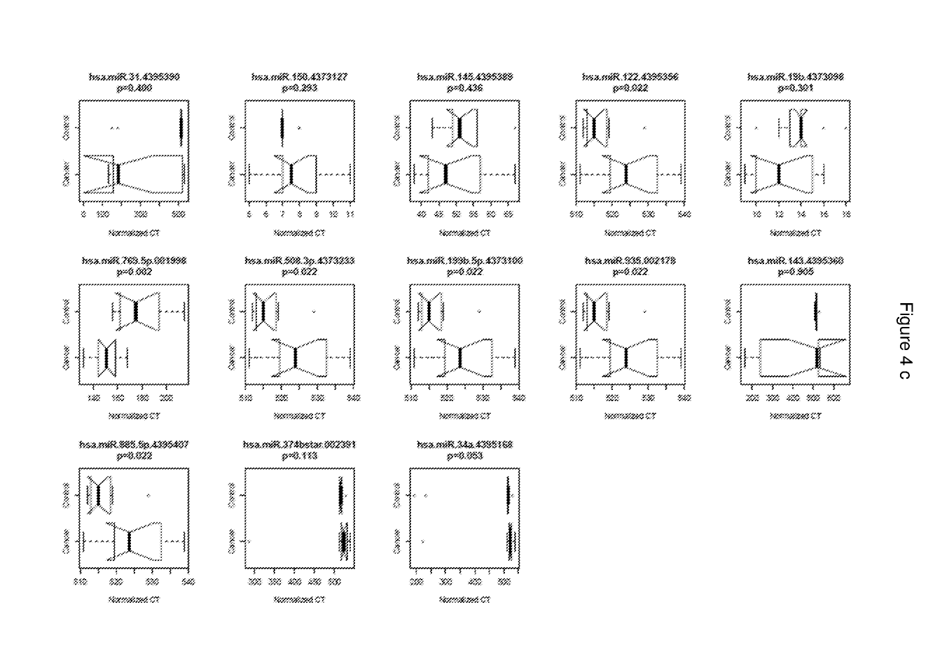

In this section the 20 samples from the pilot study are considered. There are 10 cancer cases and 10 controls and each Paxgene sample is measured on the LDA-card platform as in the discovery study. There are however differences, e.g., some of the significant miRs from the discovery study are not measured or not measurable in the pilot samples. Thus, the developed models/profiles cannot be used to evaluate the pilot samples. Since the models developed above cannot be used, boxplots and Wilcoxon (Mann-Whitney) tests are given for each significant miR in FIG. 4 a-e. In FIG. 4c for example boxplots for the pilot samples, which have been rank, are given for each of the significant miRs for the rank normalized discovery samples. The p-value above each plot corresponds to the p-value from the Wilcoxon test. It can also be seen that several miRs are missing (except for rank normalization, although it is noted that several of rank normalized miRs have large values which indicate a large proportion of missing values). The pilot samples indicate that the following miRs are significant (using p=0:05 as threshold):
Endogene controls: miR-let-7g, miR-636
Quantile: miR-769.5p, miR-93, miR-636
Rank: miR-122, miR-769.5p, miR-508.3p, miR-199b.5p, miR-935, miR-885.5p, miR-34a
120 most expressed: miR-223, miR-769.5p
Raw values: miR-144*, miR-145, miR-769.5p, miR-126*

### Example 3

### microRNA as Biomarkers in Serum for Detection of Pancreatic Cancer

BACKGROUND: Biomarkers for early diagnosis of patients with pancreatic cancer (PC) are urgently needed. The aim was to identify combinations of miRNAs with serum CA 19.9 for early diagnosis of PC.

METHODS: 417 patients with PC were included from two prospective biomarker studies from Denmark (n=306) and Germany (n=111). Controls were 59 patients with chronic pancreatitis (CP), 33 patients with other types periampullary cancers (PAC) and 248 healthy subjects (HS). MiRNA expression in pretreatment serum samples was investigated in three independent cohorts: “Discovery Study” (PC n=133, CP n=21, HS n=51); “Training Study” (PC n=198, CP: n=31, HS n=153); and “Validation Study” (PC n=86, PAC n=33, CP n=7, HS n=44). RNA was extracted from 200 μl serum from each participant. TaqMan® Human MicroRNA assay was used to screen 754 miRNAs in samples from the “Discovery Study”. Fluidigm BioMark™ PCR System was used in the “Training Study” and “Validation Study” and also tested in the cohort from “Discovery Study”.

RESULTS: The “Discovery Study” demonstrated that 34 miRNAs (out of a total of 754 miRNAs) in serum were significantly deregulated between patients with PC and controls. These miRNAs were tested in the “Training Study” and four diagnostic indexes were constructed including 5-12 miRNAs to identify patients with PC from HS and CP. These indexes used the following miRNAs in different combinations: miR-16, miR-18.a, miR-20a, miR-24, miR-25, miR-27a, miR-29c, miR-30a.5p, miR-191, miR-323.3p, miR-345, and miR-483.5p.

CONCLUSIONS: We identified several diagnostic indexes using 5 to 12 miRNAs in serum which have potential clinical value in combination with serum CA 19.9 for early diagnosis of PC.

### Materials and Methods

### Participants

### BIOPAC Study:

From Jul. 1, 2008 to Oct. 18, 2012 306 patients with pancreatic ductal adenocarcinoma (PC) were included in the Danish multicenter BIOPAC Study “BIOmarkers in patients with PAncreatic Cancer (“BIOPAC”)—can they provide new information of the disease and improve diagnosis and prognosis of the patients?” from six hospitals in Denmark. Inclusion criteria were: 1) age over 18 years; 2) histological verified PC in a resection specimen; or 3) CT scan with pancreatic tumor and a tru-cut biopsy or fine-needle aspiration cytology (FNAC) from this primary tumor or a metastasis that shows carcinoma. Serum samples for the present study were taken before operation for resectable patients and before chemotherapy for un-resectable patients. All patients provided written informed consent and the study was approved by the Regional Ethics Committee (VEK ref. KA-20060113).

### Heidelberg Study:

From August 2003 through November 2009 pretreatment blood samples were collected from 111 patients diagnosed with PC and recruited consecutively at Department of General, Visceral, and Transplant Surgery, University of Heidelberg, Germany. Clinical eligibility criteria for inclusion were: Age over 18 years, histological confirmed PC, operated with radical intentions for PC, and adequate organ function. One patient was found non-resectable during surgery. The biomarker study was approved by the Regional Ethics Committee.

### Healthy Subjects:

248 healthy blood donors (HS) from Aalborg University Hospital

Patients with Chronic Pancreatitis:

59 patients with chronic pancreatitis (CP) were included from Herlev Hospital and Rigshospitalet.

Patients with Other Types of Periampullary Cancers (PAC):

15 patients with ampullary adenocarcinoma, 6 patients with duodenal adenocarcinoma and 12 patients with common bile duct adenocarcinoma were included from Herlev Hospital and Rigshospitalet

### Study Design

The study design was the following: 1) “Discovery Study” including 133 patients with PC, 21 patients with CP, and 51 HS; 2) “Training Study” including 198 patients with PC, 31 patients with CP and 153 HS; and “Validation Study” including 86 patients with PC, 33 patients with PAC, 7 patients with CP, and 44 HS.

Purification of miRNA in Serum

All serum samples were thawed on ice and 200 μl of each sample was transferred to a tube containing 750 μl TRI Reagent BD (Molecular research Center, Inc., Cincinnati, USA) and 20 μl 5 mold acetic acid. Five microliters of synthetic RNA oligonucleotides (50 pmol/l) (Qiagen) was spiked into each sample as a control after initial serum RNA isolation (31). The total RNA was isolated using the TRI Reagent BD following the manufacturer's protocol. Each obtained total RNA pellet was resuspended in 40 μl nuclease-free water and stored at −80° C. The purification was performed at the biotech company AROS, Applied Biotechnology A/S, Denmark.

### MiRNA Expression Analysis

All miRNA analysis were performed at the biotech company AROS, Applied Biotechnology A/S, Denmark.

### “Discovery Study”.

The TaqMan® Human MicroRNA assay using A LDA cards (v2.0) and B LDA Cards (v3.0) (Part Number 4400238, Applied Biosystem) was used. This method used a set of two pre-configured micro fluidic cards that enables quantization of 754 human miRNAs. Included on each card were three TaqMan® MicroRNA assay endogenous controls to aid in data normalization and one TaqMan® MicroRNA assay not related to human as a negative control. RNA was transcribed into cDNA in two multiplex reactions each containing 3 μl of the small RNA preparation and either Megaplex RT Primer A Pool or Pool B pool and using the TaqMan® MicroRNA Reverse Transcription Kit in a total volume of 14 μl. Prior to loading of the arrays a 12 cycle pre-amplification reaction was performed using 2.5 μl cDNA in a 25 μl reaction. Each of the arrays was loaded with 800 μl Universal PCR MasterMix assay containing 1/40 of the pre-amplification reaction and run on the 7900HT Fast Real-Time PCR System. The instruction from Applied Biosystems was followed in all details including the use of pre-amplification (https://www.products.appliedbiosystems.com). Six samples could be analyzed dayly. In order to control for technical variation and nuisance factors the samples were purified in an order such that age, sex, and diagnosis were distributed in a balanced way with respect to day of RNA purification and miRNA analysis and randomized with each day. An extra serum sample, in ten replicates, was included as internal control.

### “Training Study”.

This study included 34 hsa miRNAs, 24 selected from the “Discovery Study” and 10 from the literature were analyzed using the Fluidigm BioMark™ System.

These miRNAs were analyzed in duplicates (n=10) or triplicates (n=24). Two miRNAs, mmu miR-292 and ath miR-259, were included as internal controls. The system can perform 2,304 simultaneous real-time PCR experiments running gold-standard TaqMan® assays in nanolitre quantities. The instructions from Fluidigm were followed in all details (https://www.fluidigm.com). The samples were balanced and randomized similarly to the “Discovery Study” with the exception, that the samples were balanced with respect to plates instead of analysis days. It was possible to measure 90 different serum samples on each card, implying that normalization could be achieved by using internal controls. 720 different samples were analyzed on 8 cards. In the “Training Study” the 198 patients with PC, 31 patients with chronic pancreatitis and 153 healthy subjects were analyzed but also the study population tested in the “Discovery Study—Fluidigm” (133 patients with PC, 21 patients with CP, and 51 HS) in order to test for differences between results obtained using the LDA cards and the Fluidigm method. Forty-eight serum samples from a healthy subject were measured for determination of assay reproducibility.

### “Validation Study”:

Thirteen miRNAs were selected based on the results of both the “Discovery Study” and “Training Study”, and including the miRNAs used in the different diagnostic index. Each miRNA was replicated 6 times, and analyzed using the Fluidigm BioMark™ System as described above.

### Statistical Analysis

The statistical software R version 2.15.0 was used for all analysis.

### “Discovery Study”.

Data was checked for outliers and samples with low RNA yield or absorbance were excluded. The LDA card contains one sample per card, implying that sample and card is completely confounded. In order to test the sensitivity of results due to plate variation, analysis was done on normalized values. Raw CT-values of each miRNA were normalized using either: 1) rank normalization; 2) quantile normalization; or 3) endogene normalization. Rank normalization was done for each patient in order to rank the CT-values for the miRNAs such that the lowest CT-value gets rank 1 and so on. Normalization by endogene controls or by quantile normalization expressed miRNA were done by subtracting the mean value of the endogene controls (Mamm and U6) or the mean value of the 120 most expressed miRNAs for each patient from the CT-values, respectively. Normalized data were inspected for outliers and potential technical bias from sample quality, sample purification date and array batch. For each normalization method, association between miRNA expression and case-control status was analyzed univariately by means of logistic regression. Based on the univariate analysis all miRNAs that met the 0.001 significance level were included in a multivariate model which was then reduced by means of backwards elimination and the Akaike's Information Criterion (AIC). In this analysis only complete cases were included, i.e. subjects with at least a miRNA with missing value were excluded from the analysis. The estimated univariate effects of the miRNAs in the final model were presented with odds ratio (OR) and 95% confidence intervals (CI).

### “Training Study”.

Data was checked for outliers and samples with low RNA yield or absorbance were excluded from further analysis. MiRNAs which were found significantly differentially expressed between PC and controls by more than one of the normalization methods in the “Discovery Study” were analyzed in triplicates. MiRNAs that only were found significantly differentially expressed by one of the normalization methods were analyzed in duplicates. Association between miRNA expression and case-control status was estimated univariately by means of logistic regression. The estimated effects of the 34 selected miRNAs were presented with or per unit CT increase and 95% CI. Based on the univariate analysis, all miRNAs that met the 5% significance level was included in a multivariate model which was then reduced by means of backwards elimination and the AIC criteria. A sensitivity analysis was done in order to evaluate how to handle the missing CT expression of the selected miRNAs before their inclusion in the multivariate analysis. We decided to consider a maximum number of missing values equals to 80 or 70 for each miRNA (control group forms by CP and HS combined or only HS, respectively), i.e. we restricted additionally the list of miRNAs for the multivariate analysis. Then for each of the miRNAs that satisfied this restriction, we imputed the missing values with its 0.95 quantile.

### “Validation Study”.

The repeatability of each miRNA was estimated based on the 6 replicates, and association between repeatability and Ct expression was investigated in range-mean plots. Association between miRNA expression and case-control status was estimated univariately by means of logistic regression. The estimated effects of the 13 miRNAs were presented with or per unit CT increase and 95% CI.

### Diagnostic Index.

Based on the miRNAs that were found significant in the “Training Study” four diagnostic indexes (DIs) were identified (cf. Table 13B): two indexes related to differences between patients with PC and HS, and two indexes related to differences between patients with PC and CP and HS combined. Two indexes (sPANmiRC I and sPANmiRC III; cf. table 13B) are a linear combination of selected miRNAs in such a way that technical variation is eliminated, i.e. theoretically the DI is independent of measurement platform. The remaining two indexes (sPANmiRC II and sPANmiRC IV) represent the best fit that can be achieved by using all miRNAs of the associated final multivariate model. For all four indexes, we also tested the performance by including CA 19.9 in each index. This was done by including the index as an offset in a logistic regression model together with the log(CA 19.9). In addition, we considered the performance of CA 19.9 alone.

The biomarker serum CA 19.9, used in combinations with the four DIs, was partially or totally measured in the group of healthy subjects of “Training study” and “Discovery study—Fluidigm”, respectively. Thus, we decided to impute the missing values for the biomarker as following. In “Training study”, we randomly imputed to the missing values one of the CA 19.9 value from the healthy subjects that had the measurement for the biomarker, considering a distinction by gender for the imputation and possible replacement of the same value. In “Discovery study—Fluidigm”, since all serum CA 19.9 values were missing we had to select the CA 19.9 values from healthy subjects of a cohort used in another study on pancreatic cancer, and then we randomly imputed the values from this list of values, considering a distinction by gender for the imputation and possible replacement of the same value. All statistical tests were two-sided and performed by means of Wald test. Sensitivity, specificity, ROC curve and area under the ROC curve (AUC) for each index were presented.

### Results

### Patient Characteristics

There was no significant difference in the distribution of age and sex between the “Discovery Study”, the “Training Study” and the “Validation Study”.

Association of miRNAs in Serum with Pancreatic Cancer

### “Discovery Study”.

24 miRNAs were found to have a potential to separate PC from controls (i.e. HS and CP) by at least one of three normalization methods (Table 10). Thirteen miRNAs had decreased expression (high Ct-value) in patients with PC compared to controls: miR-24, miR-25, miR-26a, miR-26b, miR-27a, miR-29c, miR-30a.5p, miR-106a, miR-191, miR-195, miR-323.3p, miR-485.3p, and miR-590.5p. Eleven miRNAs had increased expression (low Ct-value) in patients with PC compared to controls: let7b, miR-30e.3p, miR-148a, miR-185, miR-212, miR-320, miR-345, miR-483-5p, miR-618, miR-638, and miR-645.

### “Training Study”.

Of the 34 miRNAs selected from the Discovery Study (24) and the literature (10), 23 miRNAs could be validated in the Training study using univariate logistic regression with a p-value <0.05 (Table 11—1^{st} column for patients with PC compared to patients with CP and HS combined, and Table 12—1^{st} column for patients with PC compared to HS). Multivariate logistic regression including the significant miRNAs and serum CA 19.9 demonstrated that miR-16, miR-20a, miR-25, miR-27a, miR-30a.5p, miR-99a, miR-195, miR-483.5p and miR-618 were independent biomarkers for diagnosis of PC compared to HS and CP combined (Table 11—3^{rd} column). Multivariate logistic regression including the significant miRNAs and serum CA 19.9 demonstrated that miR-16, miR-20a, miR-24, miR-25, miR-30a.5p, miR-30e.3p, miR-106a, miR-195, miR-345, and miR-483.5p were independent biomarkers for diagnosis of PC compared to HS (Table 12—3^{rd} column).

### Diagnostic Index

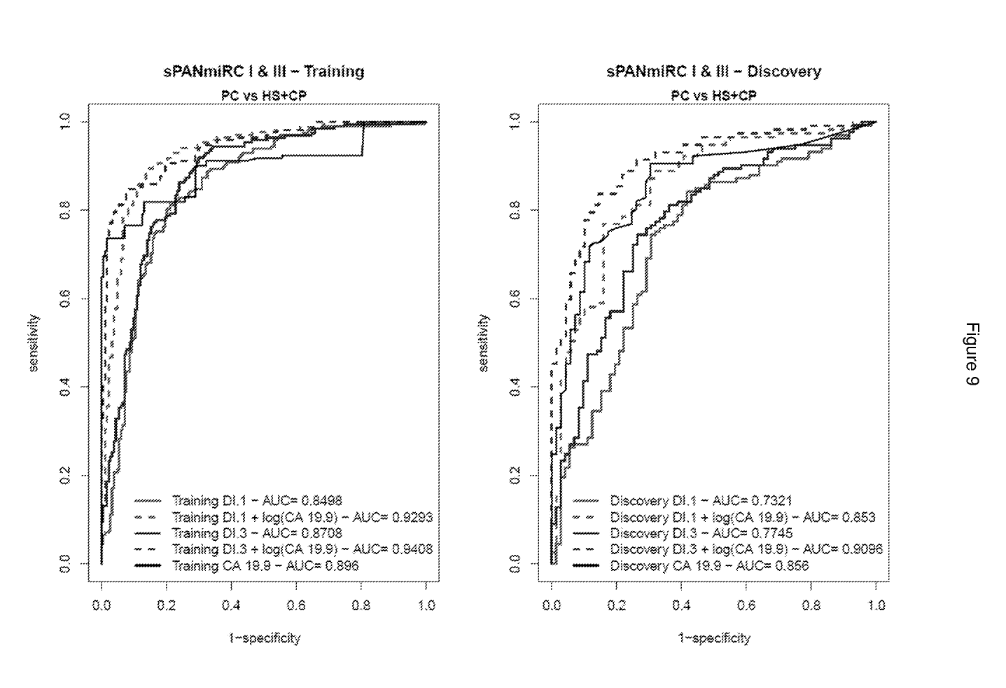

Table 13 A gives eight and Table 13 B gives four different diagnostic indexes developed according to two different cohorts: patients with PC compared to patients with CP and HS combined; and patients with PC compared to HS. FIG. 9 gives the ROC-curves using the ‘final’ four indexes of Table 13 B either alone or combined with serum CA 19.9 in the “Training Study” and the “Discovery Study—Fluidigm method”. The corresponding sensitivity, specificity, and AUC of these diagnostic indexes in the “Discovery study—Fluidigm method” and “Training study” are given in Table 14. The diagnostic accuracies were improved using the combination of high serum CA 19-9 with each sPANmiRC index.

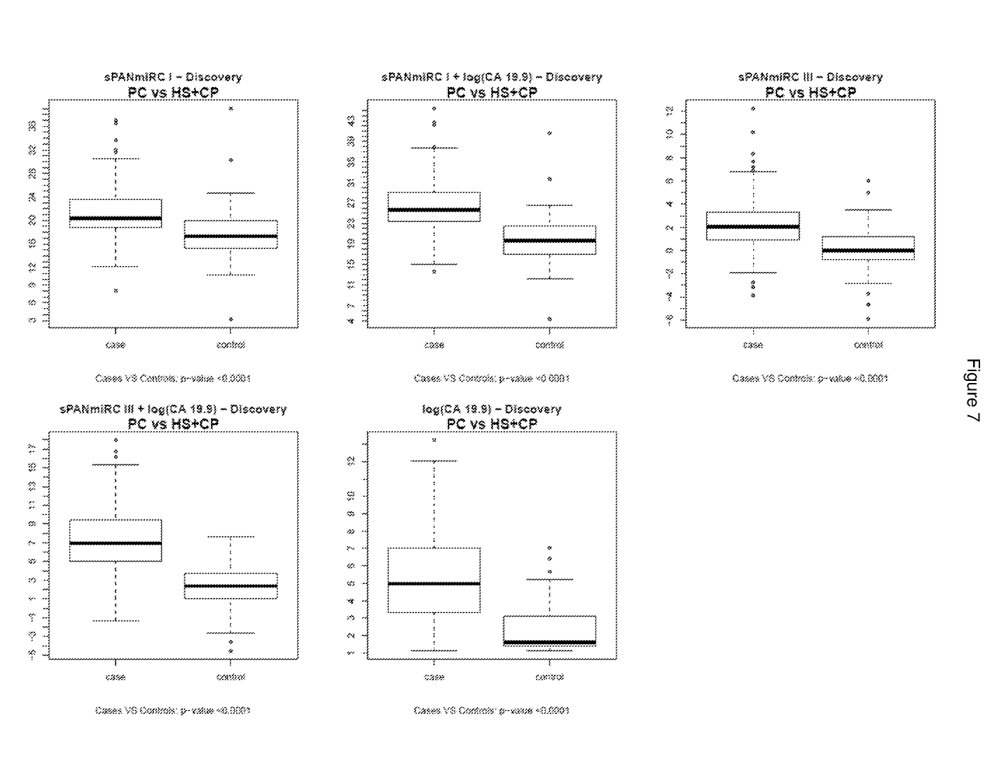

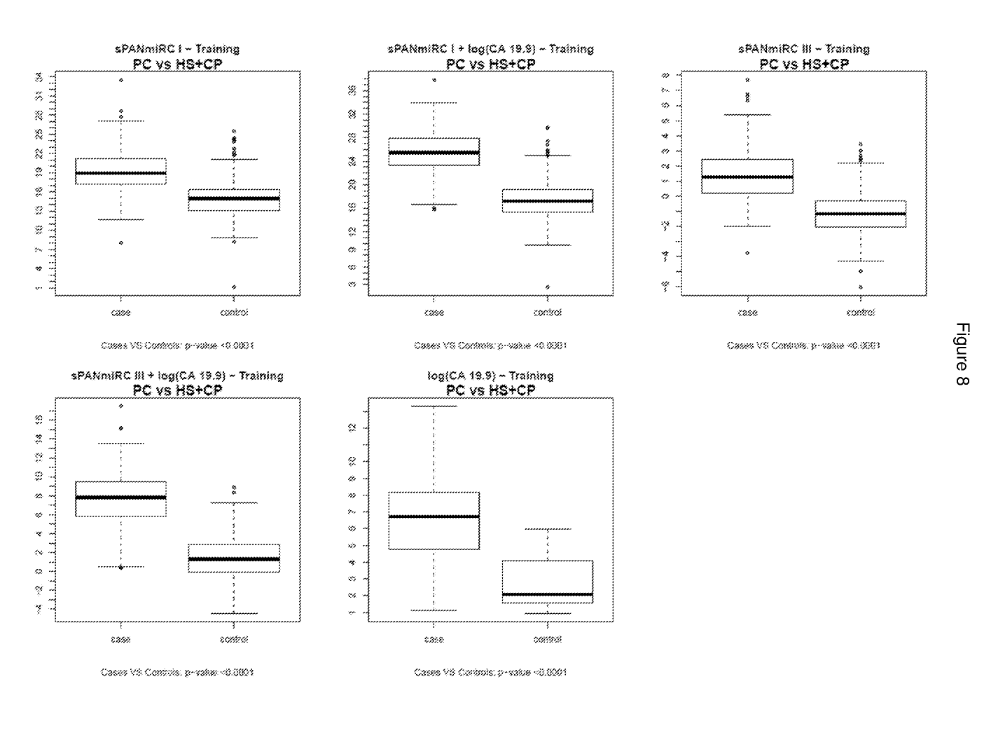

FIGS. 7 and 8 illustrates the box-plots using these four sPANmiRC indexes either alone or combined with serum CA 19.9 in the “Training Study” and the “Discovery Study—Fluidigm method” according to PC (all stages combined) or controls (CP and HS combined).

### Validation Study

13 miRNAs (miR-16, miR-20a, miR-24, miR-25, miR-27a, miR-29c, miR-30a.5p, miR-30e.3p, miR-185, miR-195, miR-323.3p, miR-345, miR-483.5p) that met the 0.05 significance level in both the “Discovery Study” and “Training Study” data, were tested in the Validation study.

### Discussion

There is a big need for better diagnostic biomarkers for early detection of patients with PC. We have identified a panel of miRNAs with expression in serum (miR-16, miR-18.a, miR-20a, miR-24, miR-25, miR-27a, miR-29c, miR-30a.5p, miR-191, miR-323.3p, miR-345, and miR-483.5p) that can be combined in four novel Diagnostic Indexes (sPANmiRC index I-IV) for diagnosis of PC. If these indexes were combined with serum CA 19-9 the diagnostic sensitivity and specificity increased.

The strengths of our study are the large number of patients and controls included in the “Discovery study” and “Training study”, the large number of miRNAs analyzed in the “Discovery Study” for selection of candidates for the diagnostic index, and the validations of the different diagnostic indexes. In the “Discovery Study” we used three different statistical methods for normalization. In order to control for technical variation and nuisance factors in our study the samples were purified and analyzed in an order such that age, sex, diagnosis were distributed in a balanced way with respect to day of purification and plate and furthermore randomized within each day and plate.

In our large “Discovery Study” testing 754 different miRNAs in 205 subjects we found 24 deregulated miRNAs in serum (let7b, miR-24, miR-25, miR-26a, miR-26b, miR-27a, miR-29c, miR-30a.5p, miR-30e.3p, miR-106a, miR-148a, miR-185, miR-191, miR-195, miR-212, miR-320, miR-323.3p, miR-345, miR-483.5p, miR-485.3p, miR-590.5p, miR-618, miR-638, miR-645) from patients with PC compared with HS and patients CP combined. Five of these diagnostic miRNAs have already been described in the literature (let7b, miR-24, miR-25, miR-185, miR-191).

Twelve miRNAs could be combined in four different diagnostic indexes (sPANmiRC index I-IV). Using our “Training study” (miR-16, miR-18.a, miR-20a, miR-24, miR-25, miR-27a, miR-29c, miR-30a.5p, miR-191, miR-323.3p, miR-345, and miR-483.5p) and validated in our cohorts of patients with PAC, CP and HS.

Purification of RNA in serum gives a low yield of miRNA compared to whole blood and it is important that standard operating procedures are used to collect the serum samples. Others have described several pre-analytical and analytical challenges in analyzing circulating miRNA.

Potentially some of these diagnostic sPANmiRC indexes can be combined in a low cost and fast PCR assay in order to select patients with unspecific symptoms like dyspeptic symptoms or unexplained weight loss for CT-scans.

In summary, we identified a panel of miRNAs in serum from a large number of patients with different stages of PC, which differentiates PC patients from patients with CP and HS. It could have considerable clinical value for diagnosing PC at an early stage, and thereby increasing the number of patients with PC who could be radically operated with curative intent.

### Example 4

### MicroRNA Biomarkers in Whole Blood for Detection of Pancreatic Cancer

A sensitive and specific diagnostic non-invasive blood test for PC would be very valuable, since it can be difficult to get useful biopsies of PC tissue from subjects suspected of having PC. Small and retrospective studies have demonstrated that high expression in plasma or serum of miR-16, -18a, -20a, -21, -24, -25, -99a, -155, -181a, -181b, -185, -191, -196a and miR-210 and low expression of let-7 family and miR-146a could identify PC from healthy subjects. However, most of these miRNAs are not validated in independent large case-control studies. Whole blood-derived miRNA profiles are suggested as new tool for early detection of PC, ovarian, lung, breast and colorectal cancer.

The advantage of whole blood is the higher miRNA-content, elimination of methodological problems related to handling of serum and plasma samples and the possibility to measure both tumor secreted miRNA, but also the changes in the miRNA profiling following the “host-reaction” in the body of patients with cancer.

There is a great need for biomarkers for early diagnosis of patients with PC to improve their poor prognosis. The aims were 1) to describe differences in miRNA expression in whole blood between patients with PC, healthy controls (HS) and patients with chronic pancreatitis (CP) and 2) to identify a panel of miRNAs (bPANmiRC index) for early diagnosis of PC. The present example elaborates further on Example 2.

### Methods

### Participants

### BIOPAC Study:

From Jul. 1, 2008 to Oct. 18, 2012 306 patients with pancreatic ductal adenocarcinoma were included in the Danish multicenter BIOPAC Study “BIOmarkers in patients with PAncreatic Cancer (“BIOPAC”)—can they provide new information of the disease and improve diagnosis and prognosis of the patients?” from six hospitals in Denmark. Inclusion criteria were age over 18 years and (1) histological verified PC (pancreatic ductal adenocarcinoma) in a resection specimen; or 2) CT scan with pancreatic tumor and a tru-cut biopsy or fine-needle aspiration cytology (FNAC) from this primary tumor or a metastasis that shows carcinoma. Blood samples for the present study were taken before operation for respectable patients (n=44) and before chemotherapy for un-respectable patients (n=365). All patients provided written informed consent and the study was approved by the Regional Ethics Committee (VEK ref. KA-20060113).

### Controls.

Healthy blood donors (n=312) from Aalborg University Hospital and patients with chronic pancreatitis (CP, n=25) included in BIOPAC Study. There was no significant difference in the distribution of age and sex between the patients included in the “Discovery Study”, “Training Study” and “Validation Study”.

### Study Design

Blood samples in Paxgene RNA tubes were allocated in chronological order to: 1) “Discovery Study” (143 patients with PC, 18 patients with chronic pancreatitis, and 69 healthy subjects; 2) “Training Study” (180 patients with PC and 170 healthy subjects) and 3) “Validation Study” (86 patients with PC, 7 patients with chronic pancreatitis, 44 healthy subjects and 33 patients with other types of upper gastrointestinal cancer (15 patients with ampullary adenocarcinoma, 6 patients with duodenal adenocarcinoma and 12 patients with common bile duct).

Purification of miRNA in Whole Blood

Pretreatment whole blood samples (2.5 ml) were collected in PAXgene Blood RNA tubes (Qiagen), which stabilize the RNA, and treated according to the manufacturer's instructions. Small RNAs were extracted from the PAXgene Blood RNA tubes in two fractions (27). The PAXgene Blood RNA tubes were processed on the Biorobot MDx (Qiagen, Hilden, Germany) using a customized protocol that binds large RNAs and rescues the run-through from the RNA binding plate. The binding condition in the run-through was subsequently modified enabling the miRNA to be purified on an RNeasy-96 μlate. The concentration of the small RNA fractions was assessed by absorbance spectrometry on a DTX 880 (Beckman Coulter). The purification was performed at the biotech company AROS, Applied Biotechnology A/S, Denmark.

### MiRNA Expression Analysis

All miRNA analysis were performed at the biotech company AROS, Applied Biotechnology A/S, Denmark.

### “Discovery Study”.

The TaqMan® Human MicroRNA assay using A LDA cards (v2.0) and B LDA Cards (v3.0) (Part Number 4400238, Applied Biosystem) was used. This method used a set of two pre-configured micro fluidic cards that enables quantization of 754 human miRNAs. Included on each card were three TaqMan® MicroRNA assay endogenous controls to aid in data normalization and one TaqMan® MicroRNA assay not related to human as a negative control. RNA was transcribed into cDNA in two multiplex reactions each containing 3 μl of the small RNA preparation and either Megaplex RT Primer A Pool or Pool B pool and using the TaqMan® MicroRNA Reverse Transcription Kit in a total volume of 14 μl. Prior to loading of the arrays a 12 cycle pre-amplification reaction was performed using 2.5 μl cDNA in a 25 μl reaction.

Each of the arrays was loaded with 800 μl Universal PCR MasterMix assay containing 1/40 of the pre-amplification reaction and run on the 7900HT Fast Real-Time PCR System. The instruction from Applied Biosystems was followed in all details including the use of pre-amplification (https://www.products.appliedbiosystems.com). Six samples could be analyzed in a day and hence the duration of the experiment was 47 days. In order to control for technical variation and nuisance factors the samples were purified in an order such that age, sex, and diagnosis were distributed in a balanced way with respect to day of RNA purification and miRNA analysis and randomized with each day. An extra whole blood sample, in ten replicates, was included as internal control.

### “Training Study”:

39 miRNAs, 38 selected from the “Discovery Study” and one (miR-18a) from the literature were analyzed using the Fluidigm BioMark™ System. These miRNAs were analyzed in duplicates (n=24) or triplicates (n=15) at AROS. This system can perform multiple simultaneous real-time PCR experiments running gold-standard TaqMan® assays in nanolitre quantities. The instructions from Fluidigm were followed in all details (https://www.fluidigm.com). The samples were balanced and randomized similarly to the “Discovery Study” with the exception, that the samples were balanced with respect to plates instead of analysis days. It was possible to measure 90 samples on each card, implying that normalization could be achieved by using internal controls.

### “Validation Study”:

Thirteen miRNAs were selected based on the results of both the Discovery and Training study, and including the miRNAs used in the Diagnostic indexes. Each miRNA was replicated 8 times (except let-7g, 7 times), and analyzed using the Fluidigm BioMark™ System.

### Statistical Analysis

The statistical software R version 2.14.0 was used including the package survival version 2.36-10 for fitting the Cox proportional hazard model and the library stats version 2.14.0 for the logistic regression model.

### “Discovery Study”.

Data was checked for outliers and samples with low RNA yield or absorbance were excluded. The LDA card contains one sample per card, implying that sample and card is completely confounded. In order to test the sensitivity of results due to plate variation, analysis was done on raw values as well as normalized values. Raw CT-values of each miRNA were pre-processed using either: 1) rank normalization; 2) quantile normalization; 3) 120 most expressed microRNAs; 4) endogene normalization; or 5) kept as raw values. Rank normalization was done for each patient in order to rank the CT-values for the miRNAs such that the lowest CT-value gets rank 1 and so on. Normalization by endogene controls or by 120 most expressed miRNA were done by subtracting the mean value of the endogene controls (mean of RNU44 and RNU48) or the mean value of the 120 most expressed miRNAs for each patient from the CT-values, respectively. Normalized data were inspected for outliers and potential technical bias from sample quality, sample purification date and array batch. The distribution of each miRNA in patients stratified according to sex, age, and diagnosis was tested by Wilcoxon rank sum test. This gives for each outcome a set of p-values, one for each miRNA, which were tested against a uniform distribution using a Kolmogorov-Smirnov test. Potential miRNAs were identified as being significant for all five normalization methods using a significance level of 0.05. The following described statistical analysis was applied separately for each normalization method. Association between miRNA expression and case-control status was analyzed univariately by means of logistic regression (25-26,29-31,34,38). Based on the univariate analysis all miRNAs that met the 0.01 significance level was included in a multivariate model which was then reduced by means of backwards elimination and the Akaike's Information Criterion (AIC). In this analysis only complete cases were included. The final model was obtained by backwards elimination of the multivariate model using AIC. The estimated effects of the most significant miRNAs were presented with 95% confidence intervals (CI).

### “Training Study”.

Data was checked for outliers and samples with low RNA yield or absorbance were excluded from further analysis. MiRNAs which were found significantly differentially expressed between PC and controls by more than one of the normalization methods in the “Discovery Study” were analyzed in triplicates. MiRNAs that only were found significantly differentially expressed by one of the normalization methods were analyzed in duplicates. The influence on reproducibility from plates and order of purification was analyzed by general linear models. Repeatability was estimated based on the repeated samples and the association between repeatability and Ct expression was investigated in range-mean plots. Association between miRNA expression and case-control status was estimated univariately by means of logistic regression, and effect estimates were compared with the ones found in the “Discovery Study”. The estimated effects of the 39 selected miRNAs were presented with 95% CI. Based on the univariate analysis all miRNAs that met the 5% significance level was included in a multivariate model which was then reduced by means of backwards elimination and the AIC criteria (only complete cases were included).

### “Validation Study”.

The repeatability of each miRNA was estimated based on the 6-7 replicates, and association between repeatability and Ct expression was investigated in range-mean plots. Association between miRNA expression and case-control status was estimated univariately by means of logistic regression. The estimated effects of the 13 miRNAs were presented with 95% CI.

Diagnostic Indexes (bPANmiRC I and II).

Based on the miRNAs that were found significant in both the “Discovery Study” and “Training Study”, we suggested two diagnostic indexes. This was based on a linear combination of selected miRNAs in such a way that technical variation was eliminated, i.e. theoretically these diagnostic indexes were independent of measurement platform. The suggested diagnostic index I for sample k is: P.I.ₖ=miRxxxₖ+miRyyyₖ−miRzzzₖ−miRwww_{k.}

As is seen from this equation no parameters were estimated from data, although the selection of the four miRNAs were based on the combined results from the “Discovery Study”, and “Training Study”, both in terms of significance and direction of estimated effects. The main idea is to cancel out technical variation by having a balanced sum of signs with plusses for miRNAs with OR>1 and minuses for miRNAs with OR<1. Based on the univariate analyses all miRNAs meeting the p<0.05 cut-off was included in a multivariate model (missing values in a miRNA were substituted with the 95% quantile for that miRNA). A backwards elimination procedure was applied using Akaike's Information Criterion (AIC) as optimality criterion to obtain a second index.

The index is the linear predictor from the logistic regression model. The computer generated index (index II) is given as

P.I.ₖ=c+Σᵢ₌₁^{p}βᵢ*miR_{i,k}=c+β₁*miR_{1,k}+β₂*miR_{2,k}+ . . . +βₚ₋₁*miR_{p-1,k}+βₚ* miR_{p,k}

If a miRNA had a missing value in a sample it was deleted from the respective miRNAs univariate analysis, and the Ct was set to the 95% quantile for the respective miRNA in calculation of the index.

Using the bPANmiRC indexes I and II the sensitivity, specificity and area under the ROC curve were presented for the “Training Study” and “Validation Study”. For all index we considered the performance by fixing the sensitivity to 0.85 to handle the difference in setup between “Training Study” and “Validation Study”,

For both index I and III we also tested the performance by including CA19-9 in the index. This was done by including the index I/III as an offset in a logistic regression model together with the log of CA.19-9. Additionally we considered the performance of CA-19.9 alone.

### Results

### MicroRNA Assay Quality

In the “Discovery Study” one sample had an absorbance (260/280)-ratio less than 1.80 and was excluded for further analysis. Three outliers (one according to many missing miRNAs and two according to a high mean Ct-value) were excluded. In the “Training Study” let-7b and miR-374b* failed to show any expression in the PCR-analysis.

Association of miRNAs in Whole Blood and Pancreatic Cancer

### “Discovery Study”.

In the multivariate analysis of the data normalized by five different methods, 38 miRNAs were found to have a potential to separate PC from controls by at least one of five normalization methods (Table 10). Fourteen of these miRNA were found by two, three or four of the normalization methods; i.e. high expression of miR-34a, miR-122, miR-145, miR-199b.5p, miR-582.3p, miR-769.5p, and miR-885.5p, and low expression of miR-31, miR-31*, miR-93, miR-126*, miR-150, miR-636, and miR-935. miR-31, miR-31*, miR-34a, miR-145, miR-150, miR-199b.5p, miR-769.5p, miR-885.5p, miR-935, are found significantly different expressed between patients with PC and controls by at least three normalization methods.

### “Training Study”.

Eighteen of the 36 miRNAs (two miRNAs were excluded) selected from the “Discovery Study” could be validated by the Fluidigm PCR in the Training population with a p-value <0.05, Table 11.

Validation of Diagnostic miRNAs Described in the Literature in Whole Blood, Serum and Plasma

Twenty-one of the selected microRNAs (determined by univariate analysis and using one of five normalization methods) or their opposite arm (from same pre-miRNA) have been described in the literature as significantly differently expressed between patients with PC and controls in either whole blood, serum or plasma. Thirty-one of the selected 38 miRNAs or their opposite arm (pre-miRNA) have a reported relation to PC described in the literature.

Diagnostic Indexes bPANmiRC I and II

Based on the results in the “Training Study” two diagnostic indexes were developed:

1) bPANmiRC I=miR-150+miR-30b−miR-145−miR-223; and

2) bPANmiRC II=6.9275−0.2134×miR-122−0.3560×miR-34a-0.8577×miR-145+1.0043×miR-636−0.6725×miR-223+0.7018×miR-26b-0.3233×miR-885.5p+1.1304×miR-150−0.2204×miR-126*−0.1730×miR-505.

bPANmiRC I had an AUC-ROC in the “Training Study” of 0.85 (95% CI: 0.81-0.89). In the “Discovery Study” the AUC-ROC was 0.88 (95% CI: 0.83-0.92) when PC was tested again HS and 0.86 (95% CI: 0.81-0.90) when PC was tested against both HS and CP. bPANmiRC II had an AUC-ROC in the “Training Study” of 0.93 (95% CI: 0.90-0.0.95). In the “Discovery Study” the AUC-ROC was 0.94 (95% CI: 0.91-0.97) when PC was tested again HS and 0.92 (95% CI: 0.89-0.95) when PC was tested against both HS and CP.

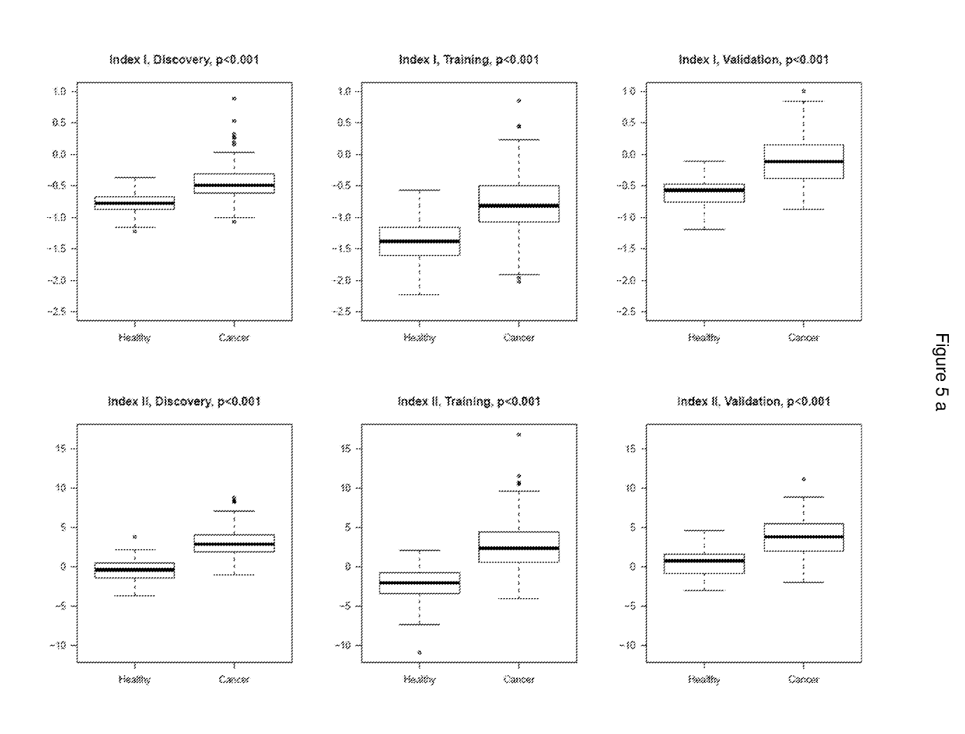

All the diagnostic accuracies were significantly improved by combining the indexes with serum CA 19-9 (Table 12). ROC-curves for bPAnmiRC I and II and the two indexes in combination with serum CA 19-9 are shown in FIG. 6. No associations between bPANmiRC I and II with age and sex were found.

### Validation Study

Thirteen miRNAs, which met the 0.05 significance level in both “Discovery Study” and “Training Study” were measured in the “Validation study” (Table 11). Ten of these miRNAs also met the significance criteria of p<0.05 in this final validation. Using bPANmiRC index I and comparing patients with PC vs. healthy subjects and chronic pancreatitis the AUC-ROC was 0.83 (95% CI: 0.76-0.89) and in combination with serum CA 19-9 the AUC-ROC was 0.92 (95% CI: 0.88-0.96). Using bPANmiRC index II and comparing patients with PC vs. healthy subjects and chronic pancreatitis the AUC-ROC was 0.81 (95% CI: 0.73-0.87) and in combination with serum CA 19-9 the AUC-ROC was 0.92 (95% CI: 0.87-0.96). Testing against a control group of both HS and CP slightly decreased the AUC for both indexes, also in the combination with serum CA 19-9 (Table 12).

Considering the sensitivity of the indexes fixed to 0.85 (see Statistics), 73 of the 86 patients with PC in the “Validation Study” had the correct diagnosis using one of the two indexes with or without CA 19-9. 2 of the 7 patients with chronic pancreatitis (29%) in the “Validation Study” had the correct diagnosis using bPANmiRC I and 2 (29%) using bPANmiRC II. 5 of the 7 patients with chronic pancreatitis (71%) in the “Validation Study” had the correct diagnosis if the bPANmiRC II was used in combination with CA 19-9. 17 of the 44 healthy subjects (39%) in the “Validation Study” had the correct diagnosis using bPANmiRC I increasing to 39 (89%) with the combination of bPANmiRC I and CA 19-9. 24 of the 44 healthy subjects (55%) in the “Validation Study” had the correct diagnosis using bPANmiRC II increasing to 39 (89%) with the combination of bPANmiRC II and CA 19-9.

### Discussion

Sensitive and specific biomarkers to identify patients with PC at an early stage are needed. This study describes two novel diagnostic indexes bPANmiRC I and II for diagnosing PC using the combination of the expression of four miRNAs (miR-30b, miR-145, miR-150, miR-223) or 10 miRNAs (miR-26b, miR-34a, miR-122, miR-126, miR-145, miR-150, miR-223, miR-505, miR-636, miR-885-5p) in whole blood. MiRNA candidates for these diagnostic indexes were selected in the “Discovery Study” testing 754 miRNAs, and the diagnostic indexes were developed using results from a “Training Study” and validated in two independent cohorts analyzed with PCR but using different platforms. Combining these two diagnostic indexes with serum CA 19-9 increased the diagnostic sensitivity and specificity compared to each of the two indexes or serum CA 19-9 (cut-off 37) used alone. The diagnostic strength was increased by letting the computer calculate an index II based on 10 miRNAs. A computer generated index may be overfitted and loose power when tested in other populations. However, our index II was validated in both the “Discovery Study” and “Validation Study” populations. The strengths of our study is the large number of patients and controls included in the “Discovery Study” and “Training Study”, the large number of miRNAs analyzed in the “Discovery Study” for selection of candidates for the DI, and the validation of the DI in two different populations using different assay platforms. In the “Discovery Study” we used five different statistical methods for normalization. Even if an array is used for these indexes, no normalization is necessary because all miRNA in the formula comes from the same array (the person's individual array), but cut-off might be changed between different analysis platforms. In order to control for technical variation and nuisance factors the samples were purified and analyzed in an order such that age, sex, diagnosis were distributed in a balanced way with respect to day of purification and randomized with each day and plate.

Our findings of significantly deregulated miRNAs in the “Discovery Study” are in good agreement with the only other study of whole blood expression profiles in PC analyzed with another array. MiR-34a, -122, -126*, -199b-5p, -223*, -374b*, -508-3p, -539, -636, -885-5p were also reported deregulated compared to healthy controls by Bauer et al. and for let-7b(let-7b*), miR-9*(-9), -23a(-23b), -26b(-26a,-26b*), -145(-145*), -582-5p(-582-3p) and -769-5p(-769-3p) they reported a closely related miRNA (shown in brackets). In serum or plasma the let-7 family and miR-24 have been reported deregulated before, and in our own setting let-7b, miR-24,-26b,-30b (instead of -30a) and -345 are deregulated in serum.

Many of the miRNAs that we find deregulated in whole blood of patients with PC have a close relation to tumor or stem cell biology. MiR-34a is related to cell cycle, differentiation and apoptosis and is regarded a key effector of the p53-tumorsuppressor function. The level of circulating miR-34a is also a marker of colorectal cancer and breast cancer. The let-7 family is deregulated in numerous types of cancer and is involved in RAS-signalling, Myc oncogene signalling pathway and JAK pathway. Purification of whole blood collected in PAXgene RNA tubes gives a higher yield of miRNA compared to serum and plasma. Circulating miRNAs found in both plasma and peripheral whole blood can originate from distant sites of tissue lesions such as solid cancers or inflammatory foci, but also from the inflammatory cells in the blood, like neutrophils, monocytes and thrombocytes, which play important roles in cancer growth, progression and metastasis, and from red blood cells (RBC). The question if miRNAs circulating in the blood can represent circulating tumor cells is still unsolved. The miRNA expression profiles in microvesicles isolated from plasma are different from the profile of isolated peripheral blood mononuclear cells (PBMC). The expression of miR-223 in plasma is highly related to neutrophil count and to lesser extent to platelet count. The expression of miR-150 in plasma is related to lymphocyte count. Given the widely held view that erythrocytes have no significant RNA content, it should be noted that mature red blood cells (RBS) bear a majority of whole blood miRNAs. Some miRNAs, such as miR-16 and miR-451 are present at more than a million-fold higher level in RBC than plasma. Levels of miRNAs expressed by RBC (miR-16, miR-92a, miR-451, miR-486) is increased 20- to 30-fold in plasma specimens undergoing hemolysis. This means that expression of some miRNAs in both whole blood and plasma samples may represent clinical characteristic as anaemia, thrombocytosis or raised neutrophils and tissue-restricted miRNAs should be given greater importance in the assessment of results. For prognostic studies it is recommended to include a complete blood count, including neutrophils. Based on the selected miRNAs our 10 miRNAs bPANmiRC II index seems more independent of the circulating blood cells than the four miRNA bPANmiRC I index where miR-150 and miR-223 have substantial weight.

Hopefully, our PI can be validated by other groups and if this is the case one could develop a fast and cheap standardized PCR analysis of miRNA in whole blood by combining these either four or 10 miRNAs for routine clinical use, eventually in combination with serum CA 19.9, in order to select patients with unspecific symptoms like dyspeptic symptoms or unexplained weight loss for CT-scans.

In summary, we identified panel of either 4 or 10 miRNAs in whole blood from a large number of patients with different stages of PC, which differentiates PC patients from patients with chronic pancreatitis and healthy subjects. If these indexes can be validate by other groups they may have considerable clinical value for diagnosing PC at an early stage, and thereby increasing the number of patients.

### REFERENCES

1. Ferlay J, Shin H-R, Bray F, Forman D, Mathers C, Parkin DM. Estimates of worldwide burden of cancer in 2008:GLOBOCAN 2008. Int J Cancer 2010; 1127: 2893-2917.
2. Siegel R, Ward E, Brawley O, Jemal A. Cancer statistics, 2011. CA Cancer J Clin 2011; 61: 212-236.
3. Ferlay J, Autier P, Boniol M, Heanue M, Colombet M, Boyle P. Estimates of the cancer incidence and mortality in Europe in 2006. Ann Oncol 2007; 18:581-592.
4. Hildago M. Pancreatic cancer. N Engl J Med 2010; 362:1605-1612.
5. Wagner M, Redaelli C, Lietz M, Seiler C, Friess H et al. Curative resection is the single most important factor determining outcome in patients with pancreatic adenocarcinoma. Br J Surg 2004; 91:586-594.
6. Locker G Y, Hamilton S, Harris J, Jessup J M, Kemeny N et al. 2006 update of recommendations for the use of tumor markers in gastrointestinal cancer. J Clin Oncol 2006; 24:5313-5327.
7. Jones S, Zhang X, Parsons D W, Lin J C-H, Leary R J, Angenendt P et al. Core signaling pathways in human pancreatic cancers revealed by global genomic analysis. Science 2008; 321:1801-1806.
8. Nelson K M, Weiss G J. MicroRNAs and cancer: past, present and potential future. Mol Cancer Ther 2008; 7:3655-3660.
9. Bartels C L, Tsongalis G J. MicroRNAs: novel biomarkers for human cancer. Clin Chem 2009; 55:623-631.
10. Iorio M V, Croce C M. MicroRNAs in cancer: small molecules with a huge impact. J Clin Oncol 2010; 27:5848-5856.
11. Visone R, Croce C M. MiRNAs and cancer. Am J Pathol 2009; 174:1131-1138.
12. Farazi T A, Spitzer J I, Morozov P, Tuschl T. MiRNAs in human cancer. J Pathol 2011; 223:102-115.
13. Rachagani S, Kumar S, Batra S K. MicroRNA in pancreatic cancer: pathological, diagnostic and therapeutic implications. Cancer Lett 2010; 292:8-16.
14. Mitchell P S, Parkin R K, Kroh E M, Fritz B R, Wyman S K, Pogosova-Agadjanyan E L et al. Circulating microRNAs as stable blood-based markers for cancer detection. PNAS 2008; 105:10513-10518.
15. Weber J A, Baxter D H, Zhang S, Huang D Y, Huang K H, Lee M J, Galas D J, Wang K. The MicroRNA Spectrum in 12 Body Fluids. Clin Chem, 2010; 56:1173-1141.
16. Wang J, Chen J, Chang P, LeBlanc A, Li D et al. MicroRNAs in plasma of pancreatic ductal adenocarcinoma patients as novel blood-based biomarkers of disease. Cancer Prev Res 2009; 2:807-813.
17. Hwang J H, Voortman J, Giovannetti E, Steinberg S M, Leon L G et al. Identification of microRNA-21 as a biomarker for chemoresistance and clinical outcome following adjuvant therapy in resectable pancreatic cancer. PLoS One 2010; 5:e10630.
18. Ali S, Almhanna K, Chen W, Philip P A, Sarkar F H. Differential expressed miRNAs in the plasma may provide a molecular signature for aggressive pancreatic cancer. Am J Transl 2011; Res 3:28-47.
19. Kong X, Du Y, Wang G, Gao J, Gong Y et al. Detection of differentially expressed microRNAs in serum of pancreatic ductal adenocarcinoma patients: miR-196a could be a potential marker for poor prognosis. Dig Dis Sci 2011; 56:602-609.
20. Liu J, Gao J, Li Z, Ren Y, Gu J, Wang X et al. Combination of plasma microRNAs with serum CA 19-9 for early detection of pancreatic cancer. Int J Cancer 2012, in press.
21. Morimura R, Komatsu S, Ichikawa D, Takeshita H, Tsujiura M, Nagata H et al. Novel diagnostic value of circulating miR-18a in plasma of patients with pancreatic cancer. Br J Cancer 2011; 105:1733-1740.
22. Liu R, Chen X, Du Y, Yao W, Shen L, Wang C, et al. Serum microRNAs expression profile as a biomarker in the diagnosis and prognosis of pancreatic cancer. Clin Chem 2012; 58, in press.
23. McShane L M, Altman D G, Sauerbrei W, Taube S E, Gion M et al. Reporting recommendations for tumor MARKer prognostic studies (REMARK). Breast Cancer Res Treat 2006; 100:229-235.
24. Kruhoffer M, Dyrskjot L, Voss T et al. Isolation of microarray-grade total RNA, microRNA, and DNA from a single PAXgene blood RNA tuber. J Mol Diagn 2007; 9:452-458.
25. Bolstad, B. Probe Level Quantile Normalization of High Density Oligonucleotide Array Data. 2001 Unpublished manuscript. http://bmbolstad.com/stuff/gnorm.pdf.
26. Bolstad, B M, Irizarry R A, Astrand M, Speed T P. A Comparison of Normalization Methods for High Density Oligonucleotide Array Data Based on Bias and Variance. Bioinformatics 2003; 19:185-193. http://bmbolstad.com/misc/normalize/normalize.html.
27. Mar J C, Kimura, Schroder K, Irvine K M, Hayashizaki Y et al. Data-driven normalization strategies for high-throughput quantitative RT-PCR. BMC Bioinformatics 2009; 10:110.
28. Bovelstad H M, Nygard S, Storvold H L, Aldrin M, Borgan O et al. Predicting survival from microarray data—a comparative study. Bioinformatics 2007; 23:2080-2087.
29. Dobson A J. An Introduction to Generalized Linear Models. London: Chapman and Hall 1990.
30. Hastie T J, Pregibon D. Generalized linear models. Chapter 6 of Statistical Models in ds J. M. Chambers and T. J. Hastie, Wadsworth & Brooks/Cole. 1992.
31. McCullagh P, Nelder J A. Generalized Linear Models. London: Chapman and Hall. 1989.
32. Cox D R. Regression models and life table (with discussion). J Royal Statistical Society series B 1972; 34:187-220.
33. Andersen P K, Gill R D. Cox's regression model for counting processes: A large sample study. Annals of Statistics 1982; 10:1100-1120.
34. Sakamoto Y, Ishiguro M, and Kitagawa G. Akaike Information Criterion Statistics. D. Reidel Publishing Company. 1986.
35. R Development Core Team. R: A Language and environment for statistical computing. R Foundation for Statistical Computing: Vienna, Austria. 2007. ISBN 3-900051-07-0.
36. Mestdagh P, Van Vp, De W A, et al. A novel and universal method for microRNA RT-qPCR data normalization. Genome Biol 2009; 10:R64.
37. Massey F Jr. The Kolmogorov-Smirnov test for goodness of fit. J Am Stat Assoc. 1951; 46(253):68-78.
38. Weber D G, Casjens S, Rozynek P, et al. Assesment of mRNA and microRNA stabilization from peripheral human blood for multicenter studies and biobanks. Biomarker Insights. 2010; 5:95-102.
39. Bloomston M, Frankel W L, Petrocca F, et al. MicroRNA expression patterns to differentiate pancreatic adenocarcinoma from normal pancreas and chronic pancreatitis. JAMA 2007; 297:1901-8.
40. Szafranska A E, Davison T S, John J, et al. expression alterations are linked to tumorigenesis and non-neoplastic processes in pancreatic ductal adenocarcinoma. Oncogene 2007; 26:4442-52.
41. Szafranska A E, Doleshal M, Edmunds H S, et al. Analysis of microRNAs in pancreatic fine-needle aspirates can classify benign and malignant tissues. Clin Chem 2008; 54:1716-24.
42. Dillhoff M, Liu J, Frankel W, Croce C, Bloomston M. MicroRNA-21 is overexpressed in pancreatic cancer and a potential predictor of survival. J Gastrointest Surg 2008; 12:2171-6.
43. Zhang Y, Li M, Wang H, et al. Profiling of 95 microRNAs in pancreatic cancer cell lines and surgical specimens by real-time PCR analysis. World J Surg 2009; 33:698-709.
44. Habbe N, Koorstra J B, Mendell J T, et al. MicroRNA miR-155 is a biomarker of early pancreatic neoplasia. Cancer Biol Ther 2009; 8(4):340-346.
45. Jiao L R, Frampton A E, Jacob J, et al. MicroRNAs targeting oncogenes are down-regulated in pancreatic malignant transformation from benign tumors. PLoS One. 2012; 7(2):e32068.
46. Jamieson N B, Morran D C, Morton J P, et al. MicroRNA molecular profiles associated with diagnosis, clinicopathologic criteria, and overall survival in patients with resectable pancreatic ductal adenocarcinoma. Clin Cancer Res 2012; 18:534-45.
47. Yu J, Li A, Hong S M, Hruban R H, Goggins M. MicroRNA alterations of pancreatic intraepithelial neoplasias. Clin Cancer Res 2012:18:981-92.
48. Bauer A S, Keller A, Costello E, et al. Diagnosis of pancreatic ductal adenocarcinomas and chronic pancreatitis by measurement of microRNA abundance in blood and tissue. PLoS One 2012; 7:e34151.
49. Schultz N A, Werner J, Willenbrock H, Roslind A, Giese N, Horn T, et al. MicroRNA expression profiles associated with pancreatic adenocarcinoma and ampullary adenocarcinoma. Modern Pathol 2012; 25:1609-22.
50. McDonald J S, Milosevic D, Reddi H V, Grebe S K, Algeciras-Schimnich A. Analysis of circulating microRNA: preanalytical and analytical challenges. Clin Chem 2011; 57:833-40.
51. McShane L M, Hayes D F. Publication of tumor marker research results: the necessity for complete and transparent reporting. J Clin Oncol 2012; 30:4223-32.
52. Altman D G, McShane L M, Sauerbrei W, Taube S E. Reporting recommendations for tumor marker prognostic studies (REMARK): explanation and elaboration. PLOS Medicine 2012; 9:e1001216.

TABLE 1
Pilot study. MicroRNAs found in the Pilot studies which
were significantly differently expressed between patients
with pancreatic cancer (PC) and controls (i.e. healthy
subjects and patients with chronic pancreatitis (CP)).
MicroRNAs in bold are those already described in the literature.

| Pilot (LDA) | Pilot (LDA) | Pilot (LDA) | | |
|---|---|---|---|---|
| Serum | Plasma | Whole blood | Pilot (Exiqon) | |
| PC = 10 | PC = 10 | PC = 10 | Serum | |
| HS = 10 | HS = 10 | HS = 10 | PC = 25 HS = 25 | Literature |
| let7b | let7b | let7 | let7a | let-7 |
| 19b | 10b* | let7a | let7c | 16 |
| 20a | 17 | let7b | let7d | 18a |
| 25 | 19a | 15p | let7d* | 20a |
| 26a | 19b | 19a | let7g | 21 |
| 26b | 20b | 22 | let7i | 24 |
| 29c | 24 | 24 | 15b | 25 |
| 30a | 27a | 25 | 17 | 99a |
| 30e-3p | 30d | 26b | 18a | 146 |
| 30c | 93 | 27a | 18b | 155 |
| 34a | 106a | 29a | 20a | 181a |
| 92a | 126 | 29c | 21 | 181b |
| 99b | 126 | 29c | 22 | 185 |
| 126 | 139-5p | 30d | 22* | 191 |
| 126 | 140-3p | 30a | 23a | 196a |
| 130b | 140-5p | 30e.3p | 23b | 210 |
| 139-5p | 146b-5p | 30c | 24 | |
| 140-5p | 146a | 92a | 26a | |
| 146a | 151-3p | 93 | 26b | |
| 150 | 151-5p | 93 | 27a | |
| 151-3p | 152 | 99b | 27b | |
| 151-5p | 186 | 125a | 30b | |
| 155 | 191 | 128 | 30c | |
| 186 | 197 | 130a | 30d | |
| 190b | 223 | 140.5p | 30e* | |
| 191 | 320 | 146a | 30e | |
| 192 | 320b | 148b | 33a | |
| 200a | 323-3p | 150 | 103 | |
| 206 | 324-3p | 151-3p | 106a | |
| 212 | 328 | 151-5p | 107 | |
| 218 | 331-3p | 185 | 126 | |
| 222 | 338-5p | 190b | 128 | |
| 223 | 340 | 191 | 130a | |
| 338-5p | 345 | 192 | 130b | |
| 345 | 374a | 193b | 140-5p | |
| 376c | 366a | 193a5p | 142-5p | |
| 378 | 376c | 194 | 146a | |
| 422a | 432 | 213 | 148b | |
| 432 | 518d | 221 | 151-3p | |
| 454 | 520d-3p | 223 | 151-5p | |
| 484 | 548a | 2965p | 152 | |
| 485-3p | 575 | 320b | 191 | |
| 516-3p | 590-5p | 3233p | 197 | |
| 518d | 652 | 3243p | 199a-3p | |
| 518f | 720 | 326 | 199a-5p | |
| 520d-3p | 8855p | 328 | 221 | |
| 520b | 12253p | 340 | 223 | |
| 548c | 1260 | 346 | 301a | |
| 548d3p | 1274b | 375 | 301b | |
| 572 | 1305 | 381 | 3245p | |
| 575 | | 425 | 328 | |
| 5763p | | 454 | 329 | |
| 5905p | | 4835p | 331-3p | |
| 601 | | 4853p | 335 | |
| 648 | | 4865p | 340 | |
| 8755p | | 5323p | 3695p | |
| 8855p | | 5743p | 374b | |
| 1208 | | 5763p | 376a | |
| 1260 | | 589 | 376b | |
| 1267 | | 5905p | 376c | |
| 1274b | | 597 | 377 | |
| 1275 | | 616 | 410 | |
| 1290 | | 625 | 4233p | |
| 1303 | | 6283p | 425* | |
| 1305 | | 636 | 431 | |
| | | 650 | 484 | |
| | | 6543p | 487b | |
| | | 660 | 4915p | |
| | | 664 | 495 | |
| | | 720 | 5743p | |
| | | 744 | 584 | |
| | | 7695p | 625* | |
| | | 942 | 652 | |
| | | 1260 | 744 | |
| | | 1274a | 766 | |
| | | 1290 | | |
| | | 1291 | | |

TABLE 2
Discovery study (example 1). MiRNAs in serum that can separate patients with
pancreatic cancer (n = 139) from healthy subjects (n = 50) and patients with chronic
pancreatitis (n = 17). Three different normalizations procedures were used.
Identified 18 miRs.

| | | | | | Endogene | |
|---|---|---|---|---|---|---|
| | Rank | | Quantile | | controls | |
| | Normalization | | Normalization | | Normalization | |
| | OR (95% CI) | | OR (95% CI) | | OR (95% CI) | |
| | decreased | | decreased | | decreased | |
| hsa MiR* | expression | P-value | expression | P-value | expression | P-value |
| miR-25 | 16 | 0.001 | 7.79 | 0.007 | | |
| | (3.45-99) | | (1.89-39) | | | |
| miR-26a | 75 | 0.003 | | | | |
| | (5.70-1840) | | | | | |
| miR-26b | 3.68 | 0.043 | | | | |
| | (1.20-15) | | | | | |
| miR-27a | | | 1.74 | 0.078 | | |
| | | | (0.97-3.41) | | | |
| miR-29c | 0.17 | 0.073 | | | | |
| | (0.02-1.03) | | | | | |
| miR-30a-5p | 3.46 | 0.027 | | | | |
| | (1.22-11) | | | | | |
| miR-106a | 0.13 | 0.048 | 0.37 | 0.058 | | |
| | (0.02-0.89) | | (0.13-1.13) | | | |
| miR-195 | 147 | <0.001 | 23 | <0.001 | 40 | <0.001 |
| | (16-24) | | (4.17-174) | | (12.24-176) | |
| miR-212 | 0.20 | 0.059 | 0.36 | 0.024 | 0.025 | <0.001 |
| | (0.03-1.01) | | (0.14-0.85) | | (0.00-0.13) | |
| miR-320 | 0.16 | 0.001 | | | | |
| | (0.05-0.46) | | | | | |
| miR-323-3p | 10.33 | <0.001 | 6.70 | 0.005 | | |
| | (3.21-43) | | (1.88-27) | | | |
| miR-345 | 9.12 | 0.010 | | | | |
| | (1.91-56) | | | | | |
| miR-483-5p | 0.097 | 0.001 | | | 0.21 | 0.011 |
| | (0.02-0.36) | | | | (0.06-0.67) | |
| miR-485-3p | | | 3.78 | 0.042 | | |
| | | | (1.12-15.24) | | | |
| miR-590-5p | | | 0.21 | 0.078 | | |
| | | | (0.033-1.07) | | | |
| miR-618 | 3.64 | 0.004 | | | | |
| | (1.58-9.42) | | | | | |
| miR-638 | 0.43 | 0.167 | | | | |
| | (0.12-1.28) | | | | | |
| miR-645 | 6.20 | 0.011 | | | 4.19 | 0.083 |
| | (1.61-28) | | | | (0.86-22.72) | |

TABLE 3
MiRNAs in whole blood that can separate patients with pancreatic cancer (n = 41)
from healthy subjects (n = 17) and patients with chronic pancreatitis (n = 4). Three
different normalizations procedures were used.

| | Rank | | Quantile | | Endogene | |
|---|---|---|---|---|---|---|
| | Normalization | | Normalization | | controls | |
| | OR (95% CI) | | OR (95% CI) | | OR (95% CI) | |
| | decreased | | decreased | | decreased | |
| hsa MiR | expression | P-value | expression | P-value | expression | P-value |
| miR-20a | | | 1.76 (1.05- | 0.065 | | |
| | | | 3.66) | | | |
| miR-30c | | | 0.40 (0.09- | 0.17 | | |
| | | | 1.29) | | | |
| miR-31 | 6.88 (2.32- | 0.003 | 3.59 (0.75- | 0.136 | 6.22 (2.26- | 0.001 |
| | 30.81) | | 23.89) | | 21.57) | |
| miR-150 | | | 3.18 (0.82- | 0.1 | | |
| | | | 14.38) | | | |
| miR-190 | 3.44 (1.58- | 0.004 | | | | |
| | 8.83) | | | | | |
| miR-196b | 21.34 (3.19- | 0.006 | | | | |
| | 275) | | | | | |

TABLE 4
Hazard Ratios (HR) and confidence intervals (CI) for miRNAs in serum
associated with overall survival in 104 patients with pancreatic cancer.

| | | | |
|---|---|---|---|
| | HR | | |
| | (per unit | Lower | Upper |
| miRNA | increase) | 95% CI | 95% CI |

Unadjusted Estimates
Overall survival (rank normalization)

| | | | |
|---|---|---|---|
| miR-27a | 1.12 | 0.98 | 1.28 |
| miR-181a | 1.08 | 0.98 | 1.19 |
| miR-511 | 1.34 | 1.09 | 1.65 |
| miR-758 | 0.90 | 0.83 | 0.98 |
| miR-362-3p | 0.70 | 0.58 | 0.84 |
| miR-100 | 1.21 | 1.05 | 1.39 |
| miR-1238 | 0.89 | 0.77 | 1.03 |

Overall survival (quantile normalization)

| | | | |
|---|---|---|---|
| miR-30e-3p | 0.36 | 0.11 | 1.11 |
| miR-99b | 2.28 | 1.13 | 4.60 |

Overall survival (endogene normalization)

| | | | |
|---|---|---|---|
| miR-19b | 1.11 | 1.03 | 1.19 |

Adjusted Estimates^{§}
Overall survival (rank normalization)

| | | | |
|---|---|---|---|
| miR-27a | 1.17 | 1.03 | 1.33 |
| miR-181a | 1.14 | 1.02 | 1.28 |
| miR-511 | 1.37 | 1.10 | 1.71 |
| miR-758 | 0.91 | 0.83 | 1.00 |
| miR-362-3p | 0.75 | 0.61 | 0.93 |
| miR-100 | 1.23 | 1.06 | 1.42 |
| miR-185 | 1.09 | 0.97 | 1.23 |

Overall survival (quantile normalization)

| | | | |
|---|---|---|---|
| miR-30e-3p | 0.30 | 0.10 | 0.92 |
| miR.331-3p | 1.40 | 1.00 | 1.97 |

Overall survival (endogene normalization)

| | | | |
|---|---|---|---|
| miR-30b | 1.12 | 1.02 | 1.23 |

^{§}Adjusted for age and gender.

TABLE 5
Hazard Ratios (HR) and confidence intervals (CI) for
miRNAs in whole blood associated with overall
survival in 41 patients with pancreatic cancer.

| | HR | | |
|---|---|---|---|
| | (per unit | Lower | Upper |
| Hsa miRNA | increase) | 95% CI | 95% CI |
| Unadjusted Estimates | | | |
| Overall survival (rank normalization) | | | |
| miR-636 | 0.95 | 0.88 | 1.01 |
| miR-150 | 1.23 | 1.03 | 1.47 |
| miR-484 | 1.93 | 0.97 | 3.84 |
| miR-370 | 1.02 | 1.00 | 1.04 |
| miR-23b | 0.98 | 0.96 | 1.01 |
| miR-450b-5p | 0.99 | 0.97 | 1.00 |
| miR-296-3p | 0.99 | 0.98 | 1.00 |
| miR-875-3p | 1.13 | 1.01 | 1.27 |

Overall survival (quantile normalization)

| | | | |
|---|---|---|---|
| miR-451 | 0.02 | 0.00 | 0.38 |
| miR-27a | 0.29 | 0.06 | 1.47 |
| miR-450a | 0.71 | 0.48 | 1.05 |

Overall survival (endogene normalization)

| miR-150 | 2.12 | 1.21 | 3.71 |
|---|---|---|---|
| miR-370 | 1.15 | 0.99 | 1.33 |
| Adjusted Estimates^{§} | | | |
| Overall survival (rank normalization) | | | |
| miR-574-3p | 0.91 | 0.83 | 1.00 |
| miR-370 | 1.04 | 1.02 | 1.07 |
| miR-324-3p | 1.26 | 1.02 | 1.56 |
| miR-874 | 1.02 | 1.00 | 1.05 |
| miR-450b-5p | 0.97 | 0.95 | 0.99 |
| miR-296-3p | 0.98 | 0.97 | 1.00 |
| miR-326 | 1.04 | 1.00 | 1.07 |
| miR-1 | 1.02 | 1.01 | 1.03 |
| miR-875-3p | 1.18 | 1.02 | 1.36 |

Overall survival (quantile normalization)

| | | | |
|---|---|---|---|
| miR-451 | 0.02 | 0.00 | 0.38 |
| miR-27a | 0.29 | 0.06 | 1.47 |
| miR-450a | 0.71 | 0.48 | 1.05 |

Overall survival (endogene normalization)

| | | | |
|---|---|---|---|
| miR-150 | 2.18 | 1.20 | 3.98 |
| miR-370 | 1.16 | 0.99 | 1.36 |

^{§}Adjusted for age and gender

TABLE 6
Discovery study, whole blood: Odds ratios (95% CI) for prediction of pancreatic cancer compared to HS and CP (cf. examples 2 and 4).

| miRNA | OR.endo | P.value.endo | OR.quan | P.value.quan | OR.rank | P.value.rank | OR.m120 | P.value.m120 | OR.raw | P.value.raw | Numbe |
|---|---|---|---|---|---|---|---|---|---|---|---|
| hsa.miR.935.002178 | 4.3037 (1.6756-11.1496) | 0.001 | 3.8378 (1.5435- | 0.009 | 13.8439 (0.0333- | <0.001 | | | 8.7219 (2.5086- | 0.001 | |
| | | | 11.718) | | 59.9235) | | | | 44.0481) | | |
| hsa.miR.885.5p.4395407 | | | 0.2203 (0.0734- | 0.004 | 0.2210 (0.0720- | 0.004 | 0.2802 (0.0952- | 0.002 | 6.2941 (0.0615- | 0.002 | |
| | | | 0.5655) | | 0.5795) | | 0.5441) | | 0.5125) | | |
| hsa.miR.769.5p.001908 | | | 0.2765 (0.1057- | 0.006 | 0.0741 (0.0217- | <0.001 | 0.1363 (0.0337- | <0.001 | 4.0574 (0.0109- | <0.001 | |
| | | | 0.6649) | | 0.1974) | | 0.3615) | | 0.2216) | | |
| hsa.miR.34a.4395168 | | | 0.3862 (0.1096- | 0.023 | 0.269 (0.1921- | 0.004 | 0.1635 (0.0609- | <0.001 | 0.1579 (0.0467- | 0.001 | |
| | | | 0.8414) | | 0.6287) | | 0.3804) | | 0.445) | | |
| hsa.miR.145.4395389 | | | 0.7026 (0.3473- | 0.231 | | <0.001 | 0.151 (0.0406- | 0.002 | 0.3916 (0.1068- | 0.013 | |
| | | | 1.0787) | | | | 0.4687) | | 0.7319) | | |
| hsa.miR.31star.002113 | 2.6402 (1.4745- | 0.002 | 0.7931 (2.5557- | <0.001 | 6.0856 (2.1167- | | | | 2.9804 (1.0742- | 0.051 | |
| | 5.0528) | | 21.5098) | | 26.2082) | | | | 9.7393) | | |
| hsa.miR31.4390396 | 6.8575 (2.4244- | <0.001 | | | 0.3520 (0.1151- | 0.002 | 2.7699 (0.9905- | 0.059 | | | |
| | 21.5335) | | | | 0.7817) | | 8.3590) | | | | |
| hsa.miR.190b.5p.4378100 | 0.4379 (0.198- | 0.023 | | | 3.1295 (1.4079- | 0.031 | 0.5063 (0.202- | 0.201 | | | |
| | 0.8659) | | | | 7.7786) | | 1.135) | | | | |
| hsa.miR150.4373127 | | | | | | 0.008 | 8.9202 (2.2116- | 0.084 | 8.7312 (2.7064- | 0.001 | |
| | | | | | | | 44.0049) | | 39.4573) | | |
| hsa.miR.03.4373302 | | | 9.3587 (2.7046- | 0.001 | | | | | 17.6024 (1.998- | 0.017 | |
| | | | 41.6883) | | | | | | 238.8499) | | |
| hsa.miR.636.4395199 | 0.253 (0.0815- | 0.012 | 3.5053 (1.3931- | 0.012 | | | | | | | |
| | 0.7019) | | 9.9597) | | | | | | | | |
| hsa.miR.582.3p.4395510 | 0.5237 (0.2888- | 0.025 | 0.5688 (0.2645- | 0.133 | | | | | | | |
| | 0.9062) | | 1.183) | | | | | | | | |
| hsa.miR.126star.000451 | 0.1291 (0.0280- | 0.005 | | | | | | | 0.112 (0.0134- | 0.032 | |
| | 0.5044) | | | | | | | | 0.7457) | | |
| hsa.miR.122.4395356 | 0.2521 (0.1298- | <0.001 | | | 0.4286 (0.1041- | 0.007 | | | | | |
| | 0.4824) | | | | 1.0001) | | | | | | |
| hsa.miR.9star.002231 | | | | | | | | | 5.0194 (1.4234- | 0.19 | |
| | | | | | | | | | 21.8376) | | |
| hsa.miR.941.002183 | | | 0.4629 (0.1977- | 0.043 | | | | | | | |
| | | | 0.9358) | | | | | | | | |
| hsa.miR.628.3p.002434 | 2.3751 (0.8917- | 0.093 | | | | | | | | | |
| | 6.8048) | | | | | | | | | | |
| hsa.miR.625.4395512 | | | 0.3951 (0.1439- | 0.049 | | | | | | | |
| | | | 0.9466) | | | | | | | | |
| hsa.miR.539.4378103 | 2.6731 (1.2843- | 0.011 | | | | | | | | | |
| | 5.9902) | | | | | | | | | | |
| hsa.miR.508.3p.4373233 | | | | | 5.7842 (1.9952- | 0.002 | | | | | |
| | | | | | 19.7082) | | | | | | |
| hsa.miR.505.4395200 | | | | | | | | | 0.3806 (0.1247- | 0.081 | |
| | | | | | | | | | 1.0814) | | |
| hsa.miR.3741.star.002301 | | | | | 2.8187 (0.9292- | 0.075 | | | | | |
| | | | | | 0.4078) | | | | | | |
| hsa.miR.362.3p.4385228 | | | 2.124 (0.8559- | 0.116 | | | | | | | |
| | | | 5.7517) | | | | | | | | |
| hsa.miR.345.4395297 | | | | | | | 4.1603 (0.4441- | 0.011 | | | |
| | | | | | | | 13.5072) | | | | |
| hsa.miR.342.5p.4395258 | | | 2.0457 (0.9329- | 0.077 | | | | | | | |
| | | | 4.6447) | | | | | | | | |
| hsa.miR.39b.4373290 | | | 0.5039 (0.247- | 0.034 | | | | | | | |
| | | | 0.8925) | | | | | | | | |
| hsa.miR.26b.4395167 | | | | | | | 0.3551 (0.1026- | 0.082 | | | |
| | | | | | | | 1.0871) | | | | |
| hsa.miR.24.2star.002441 | | | | | | | 1.9774 (0.818- | 0.146 | | | |
| | | | | | | | 5.1706) | | | | |
| hsa.miR.23a.4373074 | | | 4.0938 (1.4617- | 0.012 | | | | | | | |
| | | | 13.2346) | | | | | | | | |
| hsa.miR.223star.002098 | 0.35 (0.0004- | 0.113 | | | | | | | | | |
| | 1.2410) | | | | | | | | | | |
| hsa.miR.223.4395406 | | | | | | | 0.2103 (0.0631- | 0.007 | | | |
| | | | | | | | 0.6109) | | | | |
| hsa.miR.19b.4373098 | | | | | 2.0158 (0.3835- | 0.061 | | | | | |
| | | | | | 4.3839) | | | | | | |
| hsa.miR.186.4395396 | | | | | | | 0.2611 (0.0784- | 0.018 | | | |
| | | | | | | | 0.7335) | | | | |
| hsa.miR.144star.002148 | | | | | | | | | 8.6056 (1.9898- | 0.019 | |
| | | | | | | | | | 83.7302) | | |
| hsa.miR.143.4395360 | | | | | 2.8144 (1.0254- | 0.046 | | | | | |
| | | | | | 8.3830) | | | | | | |
| hsa.miR.128.4395327 | | | | | | | 0.177 (0.0499- | 0.001 | | | |
| | | | | | | | 0.5457) | | | | |
| hsa.let.7g.4395393 | 12.0598 (3.1718- | <0.001 | | | | | | | | | |
| | 62.5898) | | | | | | | | | | |
| hsa.let.7b.1395446 | | | | | | | | | 5.7491 (1.5283- | 0.017 | |
| | | | | | | | | | 27.8531) | | |

TABLE 7
Predicted probability of cancer (example 2)

| Patient # | Endo | Quan | Rank | X120 | Raw |
|---|---|---|---|---|---|
| 01-002 | 0.99 | 0.90 | 1.00 | 0.32 | 0.84 |
| 01-011 | 0.98 | 0.89 | 0.99 | 0.88 | 0.96 |
| 01-077 | 0.17 | 0.03 | 0.59 | 0.82 | 0.58 |
| 01-097 | 0.79 | 0.93 | 1.00 | 0.88 | 0.97 |
| 01-100 | 0.10 | 0.52 | 0.27 | 0.00 | 0.04 |
| 01-134 | 1.00 | 0.89 | 0.69 | 0.41 | 0.87 |
| 01-137 | | | 0.98 | 0.82 | |
| 01-175 | 0.74 | 0.98 | 0.92 | 0.93 | 0.99 |
| 01-177 | 0.86 | 1.00 | 1.00 | 1.00 | 1.00 |
| 01-193 | 0.94 | 1.00 | 0.96 | 0.99 | 0.98 |
| 01-200 | 0.59 | 0.79 | 0.77 | 0.78 | 1.00 |
| 01-206 | 0.83 | 0.87 | 1.00 | 0.98 | 0.97 |
| 01-209 | 0.80 | 1.00 | 0.99 | 0.92 | 1.00 |
| 01-211 | 0.28 | 0.81 | 0.00 | 0.61 | 0.61 |
| 01-214 | 0.96 | 0.99 | 1.00 | 0.99 | 1.00 |
| 01-219 | 0.58 | 0.09 | 0.04 | 0.67 | 0.01 |
| 01-224 | 0.88 | 1.00 | 1.00 | 1.00 | 1.00 |
| 01-242 | 0.99 | 1.00 | 1.00 | 0.96 | 1.00 |
| 01-257 | 1.00 | 0.99 | 1.00 | 1.00 | 1.00 |
| 01-258 | 0.83 | 0.97 | 0.72 | 0.83 | 1.00 |
| 01-271 | 0.99 | 0.99 | 1.00 | 0.96 | 1.00 |
| 01-275 | 1.00 | 0.99 | 1.00 | 1.00 | 1.00 |

TABLE 8
“Training Study” and “Validation Study”, whole blood. Odds ratios (95% CI) for prediction of pancreatic cancer
compared to controls (cf. example 4).

| | Training Study | | | | Validation Study | | | | Validation Study | | | |
|---|---|---|---|---|---|---|---|---|---|---|---|---|
| | PC vs. HS | | | | PC vs. HS + CP | | | | PC vs. HS | | | |
| | Univariate | | Adjusted | | Univariate | | Adjusted | | Univariate | | Adjusted | |
| miRNA | OR | p-value | OR | p-value | OR | p-value | OR | p-value | OR | p-value | OR | p-value |
| Let-7g | 1.04 | 0.382 | 0.70 | 0.004 | 0.74 | <0.001 | 0.74 | 0.002 | 0.70 | <0.001 | 0.59 | <0.001 |
| | (0.95-1.15) | | (0.55-0.89) | | (0.63-0.85) | | (0.61-0.89) | | (0.59-0.81) | | (0.42-0.76) | |
| miR-9* | 0.90 | 0.125 | 0.77 | 0.072 | NA | NA | NA | NA | NA | NA | NA | NA |
| | (0.79-1.03) | | (0.57-1.01) | | | | | | | | | |
| miR-18a § | 0.99 | 0.777 | 0.84 | 0.068 | NA | NA | NA | NA | NA | NA | NA | NA |
| | (0.90-1.08) | | (0.69-1.01) | | | | | | | | | |
| miR-19b | 1.08 | 0.236 | 1.23 | 0.090 | NA | NA | NA | NA | NA | NA | NA | NA |
| | (0.95-1.25) | | (0.98-1.61) | | | | | | | | | |
| miR-23a | 0.73 | <0.001 | 1.02 | 0.860 | NA | NA | NA | NA | NA | NA | NA | NA |
| | (0.64-0.82) | | (0.83-1.24) | | | | | | | | | |
| miR-24-2* | 0.95 | 0.602 | 1.20 | 0.284 | NA | NA | NA | NA | NA | NA | NA | NA |
| | (0.80-1.14) | | (0.87-1.73) | | | | | | | | | |
| miR-26b | 1.06 | 0.360 | 0.61 | 0.002 | 0.65 | <0.001 | 0.66 | <0.001 | 0.61 | <0.001 | 0.53 | <0.001 |
| | (0.94-1.19) | | (0.44-0.82) | | (0.54-0.76) | | (0.52-0.80) | | (0.50-0.73) | | (0.37-0.70) | |
| miR-30b | 1.10 | 0.278 | 1.11 | 0.387 | 0.76 | 0.064 | 0.75 | 0.151 | 0.80 | 0.150 | 0.75 | 0.265 |
| | (0.93-1.31) | | (0.86-1.43) | | (0.57-1.01) | | (0.50-1.11) | | (0.58-1.08) | | (0.46-1.24) | |
| miR-31 | 1.48 | <0.001 | 1.10 | 0.384 | 1.04 | 0.645 | 1.04 | 0.757 | 1.00 | 0.986 | 0.91 | 0.541 |
| | (1.32-1.68) | | (0.89-1.38) | | (0.87-1.25) | | (0.81-1.34) | | (0.83-1.21) | | (0.68-1.23) | |
| miR-31* | 1.00 | 0.987 | 1.05 | 0.796 | NA | NA | NA | NA | NA | NA | NA | NA |
| | (0.82-1.22) | | (0.72-1.52) | | | | | | | | | |
| miR-34a | 0.72 | <0.001 | 0.63 | <0.001 | 0.71 | 0.003 | 0.64 | 0.004 | 0.74 | 0.010 | 0.62 | 0.010 |
| | (0.63-0.81) | | (0.48-0.81) | | (0.56-0.88) | | (0.46-0.86) | | (0.58-0.92) | | (0.42-0.88) | |
| miR-93 | 1.09 | 0.181 | 0.76 | 0.068 | NA | NA | NA | NA | NA | NA | NA | NA |
| | (0.96-1.24) | | (0.56-1.02) | | | | | | | | | |
| miR-122 | 0.74 | <0.001 | 0.78 | 0.046 | 0.92 | 0.449 | 1.20 | 0.325 | 0.86 | 0.233 | 0.92 | 0.763 |
| | (0.65-0.82) | | (0.59-0.98) | | (0.73-1.14) | | (0.83-1.77) | | (0.65-1.10) | | (0.53-1.55) | |
| miR-126* | 1.01 | 0.764 | 0.71 | <0.001 | 0.71 | <0.001 | 0.71 | 0.001 | 0.67 | <0.001 | 0.63 | 0.001 |
| | (0.94-1.08) | | (0.58-0.84) | | (0.60-0.82) | | (0.57-0.85) | | (0.55-0.79) | | (0.46-0.80) | |
| miR-128 | 0.67 | <0.001 | 1.19 | 0.428 | NA | NA | NA | NA | NA | NA | NA | NA |
| | (0.54-0.83) | | (0.77-1.82) | | | | | | | | | |
| miR-143 | 0.73 | <0.001 | 0.77 | 0.008 | NA | NA | NA | NA | NA | NA | NA | NA |
| | (0.66-0.80) | | (0.63-0.93) | | | | | | | | | |
| miR-144* | 0.97 | 0.384 | 0.81 | 0.003 | NA | NA | NA | NA | NA | NA | NA | NA |
| | (0.92-1.03) | | (0.71- 0.93) | | | | | | | | | |
| miR-145 | 0.67 | <0.001 | 0.82 | 0.128 | 0.57 | 0.001 | 0.61 | 0.023 | 0.58 | 0.003 | 0.72 | 0.169 |
| | (0.58-0.78) | | (0.62-1.04) | | (0.40-0.78) | | (0.39-0.92) | | (0.40-0.82) | | (0.44-1.14) | |
| miR-150 | 1.76 | <0.001 | 1.13 | 0.370 | 2.20 | 0.001 | 1.71 | 0.051 | 3.02 | <0.001 | 2.68 | 0.011 |
| | (1.46-2.15) | | (0.85-1.50) | | (1.41-3.61) | | (1.03-3.06) | | (1.80-5.47) | | (1.30-6.01) | |
| miR-186 | 1.02 | 0.781 | 1.17 | 0.120 | NA | NA | NA | NA | NA | NA | NA | NA |
| | (0.90-1.15) | | (0.98-1.48) | | | | | | | | | |
| miR-199b-5p | 0.93 | 0.248 | 1.07 | 0.585 | NA | NA | NA | NA | NA | NA | NA | NA |
| | (0.82-1.05) | | (0.83-1.39) | | | | | | | | | |
| miR-223 | 0.49 | <0.001 | 0.71 | 0.207 | 0.59 | <0.001 | 0.62 | 0.010 | 0.60 | 0.001 | 0.62 | 0.030 |
| | (0.37-0.64) | | (0.40-1.08) | | (0.44-0.78) | | (0.43-0.89) | | (0.45-0.80) | | (0.40-0.94) | |
| miR-223* | 0.86 | 0.008 | 0.69 | 0.006 | NA | NA | NA | NA | NA | NA | NA | NA |
| | (0.77-0.96) | | (0.53-0.89) | | | | | | | | | |
| miR-342-5p | 1.28 | <0.001 | 1.34 | 0.038 | NA | NA | NA | NA | NA | NA | NA | NA |
| | (1.12-1.48) | | (1.02-1.79) | | | | | | | | | |
| miR-345 | 0.80 | 0.015 | 0.97 | 0.843 | NA | NA | NA | NA | NA | NA | NA | NA |
| | (0.67-0.96) | | (0.68-1.35) | | | | | | | | | |
| miR-362-3p | 0.99 | 0.822 | 1.22 | 0.086 | NA | NA | NA | NA | NA | NA | NA | NA |
| | (0.89-1.10) | | (0.98-1.54) | | | | | | | | | |
| miR-505 | 0.74 | <0.001 | 0.64 | 0.022 | 0.53 | <0.001 | 0.50 | 0.001 | 0.48 | <0.001 | 0.34 | <0.001 |
| | (0.62-0.86) | | (0.43-0.92) | | (0.39-0.69) | | (0.33-0.73) | | (0.34-0.64) | | (0.17-0.58) | |
| miR-508-3p | 1.15 | 0.727 | 1.20 | 0.841 | NA | NA | NA | NA | NA | NA | NA | NA |
| | (0.52-2.80) | | (0.17-14.42) | | | | | | | | | |
| miR-539 | 1.00 | 0.977 | 1.08 | 0.331 | NA | NA | NA | NA | NA | NA | NA | NA |
| | (0.92-1.08) | | (0.92-1.27) | | | | | | | | | |
| miR-582-3p | 0.82 | 0.005 | 0.83 | 0.191 | NA | NA | NA | NA | NA | NA | NA | NA |
| | (0.71-0.94) | | (0.61-1.09) | | | | | | | | | |
| miR-625 | 0.80 | <0.001 | 0.96 | 0.625 | NA | NA | NA | NA | NA | NA | NA | NA |
| | (0.73-0.88) | | (0.80-1.14) | | | | | | | | | |
| miR-628-3p | 0.99 | 0.774 | 1.00 | 0.979 | NA | NA | NA | NA | NA | NA | NA | NA |
| | (0.89-1.09) | | (0.80-1.24) | | | | | | | | | |
| miR-636 | 1.62 | <0.001 | 1.84 | <0.001 | 2.81 | <0.001 | 2.04 | 0.005 | 3.89 | <0.001 | 2.71 | 0.004 |
| | (1.39-1.92) | | (1.35-2.61) | | (1.83-4.62) | | (1.28-3.49) | | (2.32-7.23) | | (1.47-5.74) | |
| miR-769-5p | 0.80 | <0.001 | 1.06 | 0.689 | NA | NA | NA | NA | NA | NA | NA | NA |
| | (0.71-0.89) | | (0.81-1.39) | | | | | | | | | |
| miR-885-5p | 0.67 | <0.001 | 0.79 | 0.136 | 0.62 | <0.001 | 0.71 | 0.016 | 0.61 | <0.001 | 0.65 | 0.013 |
| | (0.58-0.77) | | (0.58-1.08) | | (0.49-0.78) | | (0.52-0.93) | | (0.46-0.77) | | (0.46-0.91) | |
| miR-935 | 1.23 | 0.001 | 1.17 | 0.211 | NA | NA | NA | NA | NA | NA | NA | NA |
| | (1.09-1.40) | | (0.91- 1.49) | | | | | | | | | |
| miR-941 | 0.87 | 0.254 | 0.89 | 0.609 | NA | NA | NA | NA | NA | NA | NA | NA |
| | (0.68-1.11) | | (0.47-1.36) | | | | | | | | | |

TABLE 9
Performance of bPANmiRC indexes I and II alone, CA 19-9 alone, and index I and II in combination with CA 19-9 in the differential
diagnosis of PC from HS and patients with CP. 95% confidence intervals are given in parentheses (cf example 4—whole blood)

| | | Designed | | Sensitivity | Specificity | Accuracy | AUC | True | True | Fals | False |
|---|---|---|---|---|---|---|---|---|---|---|---|
| Study | Index | Sensitivity | Cut off | (95% C.I.) | (95% C.I.) | (95% C.I.) | (95% C.I.) | pos. | neg. | pos. | neg. |
| Discovery | bPANmiRC I | 0.85 | −0.723 | 0.846 | 0.639 | 0.763 | 0.859 | 121 | 62 | 35 | 22 |
| HS + CP | | | | (0.776; 0.901) | (0.535; 0.763) | (0.704; 0.815) | (0.811; 0.904) | | | | |
| Discovery HS | bPANmiRC I | 0.85 | −0.723 | 0.846 | 0.658 | 0.779 | 0.880 | 121 | 52 | 27 | 22 |
| | | | | (0.776; 0.901) | (0.543; 0.761) | (0.719; 0.832) | (0.834; 0.922) | | | | |
| Discovery CP | bPANmiRC I | 0.85 | −0.723 | 0.846 | 0.556 | 0.814 | 0.767 | 121 | 10 | 8 | 22 |
| | | | | (0.776; 0.901) | (0.308; 0.785) | (0.745; 0.871) | (0.642; 0.871) | | | | |
| Training HS | bPANmiRC I | 0.85 | −1.272 | 0.850 | 0.633 | 0.736 | 0.847 | 153 | 126 | 73 | 27 |
| | | | | (0.789; 0.899) | (0.562; 0.700) | (0.689; 0.780) | (0.805; 0.886) | | | | |
| Validation | bPANmiRC I | 0.85 | −0.642 | 0.849 | 0.373 | 0.672 | 0.828 | 73 | 19 | 32 | 13 |
| HS + CP | | | | (0.755; 0.917) | (0.241; 0.519) | (0.586; 0.749) | (0.758; 0.891) | | | | |
| Validation HS | bPANmiRC I | 0.85 | −0.642 | 0.849 | 0.386 | 0.692 | 0.838 | 73 | 17 | 27 | 13 |
| | | | | (0.755; 0.917) | (0.241; 0.545) | (0.605; 0.770) | (0.767; 0.902) | | | | |
| Validation CP | bPANmiRC I | 0.85 | −0.642 | 0.849 | 0.286 | 0.806 | 0.764 | 73 | 2 | 5 | 13 |
| | | | | (0.755; 0.917) | (0.037; 0.710) | (0.711; 0.881) | (0.593; 0.907) | | | | |
| Discovery | CA.19.9 | NA | 37 | 0.881 | 0.918 | 0.896 | 0.872 | 126 | 89 | 8 | 17 |
| HS + CP | | | | (0.816; 0.929) | (0.843; 0.964) | (0.850; 0.931) | (0.817; 0.921) | | | | |
| Discovery HS | CA.19.9 | NA | 37 | 0.881 | 1.000 | 0.923 | 0.881 | 126 | 79 | 0 | 17 |
| | | | | (0.816; 0.929) | (0.954; 1.000) | (0.880; 0.931) | (0.825; 0.930) | | | | |
| Discovery CP | CA.19.9 | NA | 37 | 0.881 | 0.556 | 0.845 | 0.831 | 126 | 10 | 8 | 17 |
| | | | | (0.816; 0.929) | (0.308; 0.785) | (0.780; 0.900) | (0.740; 0.903) | | | | |
| Training HS | CA.19.9 | NA | 37 | 0.856 | 0.990 | 0.926 | 0.904 | 154 | 197 | 2 | 26 |
| | | | | (0.800; 0.903) | (0.964; 0.999) | (0.751; 0.950) | (0.866; 0.939) | | | | |
| Validation | CA.19.9 | NA | 37 | 0.791 | 0.882 | 0.825 | 0.892 | 68 | 45 | 6 | 18 |
| HS + CP | | | | (0.690; 0.871) | (0.761; 0.956) | (0.751; 0.884) | (0.832; 0.945) | | | | |
| Validation HS | CA.19.9 | NA | 37 | 0.791 | 0,955 | 0.846 | 0.911 | 68 | 42 | 2 | 18 |
| | | | | (0.690; 0.871) | (0.845; 0.994) | (0.772; 0.903) | (0.855; 0.960) | | | | |
| Validation CP | CA.19.9 | NA | 37 | 0.791 | 0.429 | 0.763 | 0.775 | 68 | 3 | 4 | 18 |
| | | | | (0.690; 0.871) | (0.100; 0.816) | (0.664; 0.845) | (0.661; 0.879) | | | | |
| Discovery | bPANmiRC I | 0.85 | −0.319 | 0.846 | 0.918 | 0.875 | 0.879 | 121 | 89 | 8 | 22 |
| HS + CP | and CA 19.9 | | | (0.776; 0.901) | (0.844; 0.964) | (0.826; 0.914) | (0.829; 0.927) | | | | |
| Discovery HS | bPANmiRC I | 0.85 | −0.319 | 0.846 | 0.987 | 0.896 | 0.885 | 121 | 78 | 1 | 22 |
| | and CA 19.9 | | | (0.776; 0.901) | (0.931; 1.000) | (0.849; 0.933) | (0.833; 0.933) | | | | |
| Discovery CP | bPANmiRC I | 0.85 | −0.319 | 0.846 | 0.611 | 0.820 | 0.853 | 121 | 11 | 7 | 22 |
| | and CA 19.9 | | | (0.776; 0.901) | (0.357; 0.820) | (0.752; 0.876) | (0.775; 0.924) | | | | |
| Training HS | bPANmiRC I | 1.85 | −0.409 | 0.850 | 0.960 | 0.908 | 0.927 | 153 | 191 | 8 | 27 |
| | and CA 19.9 | | | (0.789; 0.899) | (0.922; 0.908) | (0.874; 0.935) | (0.894; 0.954) | | | | |
| Validation | bPANmiRC I | 1.85 | −0.019 | 0.849 | 0.804 | 0.832 | | 73 | 41 | 10 | 13 |
| HS + CP | and CA 19.9 | | | (0.755; 0.917) | (0.669; 0.902) | (0.759; 0.891) | 0.922253 | | | | |
| Validation HS | bPANmiRC I | 1.85 | −0.019 | 0.849 | 0.886 | 0.862 | 0.938 | 73 | 39 | 5 | 13 |
| | and CA 19.9 | | | (0.755; 0.917) | (0.754; 0.962) | (0.790; 0.912) | (0.894; 0.975) | | | | |
| Validation CP | bPANmiRC I | 1.85 | −0.019 | 0.849 | 0.286 | 0.806 | 0.822 | 73 | 2 | 5 | 13 |
| | and CA 19.9 | | | (0.755; 0.917) | (0.037; 0.710) | (0.711; 0.881) | (0.733; 0.902) | | | | |
| Discovery | bPANmiRC II | 1.85 | 1.21 | 0.846 | 0.856 | 0.850 | 0.925 | 121 | 83 | 14 | 22 |
| HS + CP | | | | (0.776; 0.901) | (0.770; 0.919) | (0.798, 0.893) | (0.890; 0.955) | | | | |
| Discovery HS | bPANmiRC II | 1.85 | 1.221 | 0.846 | 0.886 | 0.860 | 0.943 | 121 | 70 | 9 | 22 |
| | | | | (0.776; 0.901) | (0.794; 0.947) | (0.808; 0.903) | (0.911: 0.967) | | | | |
| Discovery CP | bPANmiRC II | 1.85 | 1.221 | 0.846 | 0.722 | 0.832 | 0.843 | 121 | 13 | 5 | 22 |
| | | | | (0.776; 0.901) | (0.465; 0.832) | (0.765; 0.886) | (0.754; 0.920) | | | | |
| Training HS | bPANmiRC II | 1.85 | −0.311 | 0.850 | 0.854 | 0.852 | 0.927 | 153 | 170 | 29 | 27 |
| | | | | (0.789; 0.899) | (0.797; 0.852) | (0.812; 0.886) | (0.899; 0.920) | | | | |
| Validation | bPANmiRC II | 1.85 | 0.797 | 0.849 | 0.510 | 0.723 | 0.809 | 73 | 26 | 25 | 13 |
| HS + CP | | | | (0.755; 0.917) | (0.366; 0.652) | (0.640; 0.796) | (0.734; 0.873) | | | | |
| Validation HS | bPANmiRC II | 1.85 | 0.797 | 0.849 | 0.545 | 0.746 | 0.818 | 73 | 24 | 20 | 13 |
| | | | | (0.755; 0.917) | (0.388; 0.696) | (0.662; 0.818) | (0.747; 0.885) | | | | |
| Validation CP | bPANmiRC II | 1.85 | 0.797 | 0.849 | 0.286 | 0.806 | 0.754 | 73 | 2 | 5 | 13 |
| | | | | (0.755; 0.917) | (0.037; 0.710) | (0.711; 0.881) | (0.595; 0.894) | | | | |
| Discovery | bPANmiRC II | 1.85 | 0.712 | 0.846 | 0.969 | 0.896 | 0.950 | 121 | 94 | 3 | 22 |
| HS + CP | and CA 19.9 | | | (0.776; 0.901) | (0.912; 0.994) | (0.850; 0.931) | (0.921; 0.975) | | | | |
| Discovery HS | bPANmiRC II | 1.85 | 0.712 | 0.846 | 0.987 | 0.896 | 0.958 | 121 | 78 | 1 | 22 |
| | and CA 19.9 | | | (0.776; 0.901) | (0.931; 1.000) | (0.849; 0.958) | (0.930; 0.981) | | | | |
| Discovery CP | bPANmiRC II | 1.85 | 0.712 | 0.846 | 0.889 | 0.851 | 0.913 | 121 | 16 | 2 | 22 |
| | and CA 19.9 | | | (0.776; 0.901) | (0.653; 0.986) | (0.786; 0.902) | (0.858; 0.958) | | | | |
| Training HS | bPANmiRC II | 1.85 | 0.829 | 0.850 | 0.980 | 0.918 | 0.970 | 153 | 195 | 4 | 27 |
| | and CA 19.9 | | | (0.789; 0.899) | (0.950; 0.994) | (0.886; 0.944) | (0.954; 0.982) | | | | |
| Validation | bPANmiRC II | 1.85 | 0.536 | 0.849 | 0.863 | 0.854 | 0.921 | 73 | 44 | 7 | 13 |
| HS + CP | and CA 19.9 | | | (0.755; 0.917) | (0.737; 0.943) | (0.784; 0.944) | (0.872; 0.962) | | | | |
| Validation HS | bPANmiRC II | 1.85 | 0.536 | 0.849 | 0.886 | 0.862 | 0.932 | 73 | 39 | 5 | 13 |
| | and CA 19.9 | | | (0.755; 0.917) | (0.754; 0.962) | (0.790; 0.916) | (0.885; 0.971) | | | | |
| Validation CP | bPANmiRC II | 1.85 | 0.536 | 0.849 | 0.714 | 0.839 | 0.852 | 73 | 5 | 2 | 13 |
| | and CA 19.9 | | | (0.755; 0.917) | (0.2900.963) | (0.748; 0.907) | (0.766; 0.927) | | | | |

TABLE 10
“Discovery Study” (example 3—serum). Odds ratios (95% CI) for prediction of PC
(n = 133) compared to controls (ie. patients with chronic pancreatitis (n = 21) and
healthy subjects n = 51).

| | | | | | Endogene | |
|---|---|---|---|---|---|---|
| | Rank | | Quantile | | controls | |
| | Normalization | | Normalization | | Normalization | |
| | OR (95% CI) | | OR (95% CI) | | OR (95% CI) | |
| | decreased | | decreased | | decreased | |
| miRNA | expression | P-value | expression | P-value | expression | P-value |
| let.7b. | 0.9 (0.69-1.18) | 0.418 | 0.17 (0.09-0.32) | <0.001 | 0.24 (0.14-0.41) | <0.001 |
| miR-24 | 1.68 (1.16-2.54) | 0.00898 | 1.9 (1.29-2.95) | 0.00222 | 0.97 (0.6-1.58) | 0.91 |
| miR-25 | 7.08 (3.51-16.65) | <0.001 | 7.09 (3.71-15.46) | <0.001 | 1.94 (1.27-3.04) | 0.0028 |
| miR-26a | 2.18 (1.41-3.61) | 0.00108 | 2.4 (1.42-4.25) | 0.00171 | 1.27 (0.8-2.03) | 0.305 |
| miR-26b | 3.93 (2.35-7.2) | <0.001 | 4.9 (2.82-9.5) | <0.001 | 1.58 (1.02-2.48) | 0.0432 |
| miR-27a | 1.88 (1.28-2.95) | 0.00338 | 1.94 (1.45-2.67) | <0.001 | 1.04 (0.67-1.64) | 0.857 |
| miR-29c | 3.88 (2.28-7.13) | <0.001 | 3.19 (2.04-5.2) | <0.001 | 1.57 (1.06-2.36) | 0.0281 |
| miR-30a.5p | 0.35 (0.18-0.64) | 0.001 | NA | NA | NA | NA |
| miR-30e.3p | 0.97 (0.86-1.1) | 0.596 | 0.64 (0.42-0.95) | 0.0299 | 0.52 (0.34-0.79) | 0.00271 |
| miR-106a | 2.31 (1.51-3.8) | <0.001 | 2.48 (1.63-4.13) | <0.001 | 1.15 (0.72-1.85) | 0.555 |
| miR-148a | 0.26 (0.14-0.48) | <0.001 | 0.75 (0.56-1) | 0.0516 | 0.72 (0.43-1.21) | 0.209 |
| miR-185 | 1.3 (0.7-2.45) | 0.415 | 0.47 (0.29-0.75) | 0.00206 | 0.23 (0.11-0.44) | <0.001 |
| miR-191 | 1.46 (0.98-2.24) | 0.0711 | 1.65 (1.13-2.55) | 0.0157 | 0.97 (0.61-1.54) | 0.906 |
| miR-195 | 19.98 (9.01-49.79) | <0.001 | 34.03 (14.24-92.76) | <0.001 | 4.43 (2.68-7.79) | <0.001 |
| miR-212 | 0.33 (0.2-0.51) | <0.001 | 0.21 (0.12-0.33) | <0.001 | 0.19 (0.11-0.32) | <0.001 |
| miR-320 | 0.24 (0.14-0.39) | <0.001 | 0.36 (0.22-0.57) | <0.001 | 0.56 (0.33-0.93) | 0.0281 |
| miR-323.3p | 3.28 (2.05-5.48) | <0.001 | 12.12 (6.25-25.38) | <0.001 | 1.13 (0.77-1.68) | 0.536 |
| miR-345 | 0.4 (0.24-0.66) | <0.001 | 0.81 (0.59-1.08) | 0.159 | 0.61 (0.37-1) | 0.0542 |
| miR-483.5p | 0.39 (0.26-0.56) | <0.001 | 0.44 (0.29-0.65) | <0.001 | 0.35 (0.21-0.56) | <0.001 |
| miR-485.3p. | 2.5 (1.55-4.21) | <0.001 | 2.45 (1.62-3.82) | <0.001 | 1.11 (0.79-1.58) | 0.538 |
| miR-590.5p | 7.84 (4.11-16.22) | <0.001 | 8.21 (4.68-15.29) | <0.001 | 1.66 (1.11-2.54) | 0.0151 |
| miR-618 | 0.75 (0.56-0.97) | 0.0441 | 0.87 (0.77-0.97) | 0.0175 | 0.44 (0.28-0.67) | <0.001 |
| miR-638 | 0.95 (0.8-1.14) | 0.565 | 1.03 (0.75-1.41) | 0.873 | 0.44 (0.27-0.68) | <0.001 |
| miR-645 | 0.97 (0.8-1.2) | 0.772 | 0.91 (0.72-1.15) | 0.44 | 0.39 (0.23-0.63) | <0.001 |

Identical miRs compared to Table 2 (18 miRs), except 6 additional miRs are identified = 24 miRs (let7b, miR-24, miR-30e3p, miR-148a, miR-185, miR-191).

TABLE 11
‘Training study’ (example 3—serum) Univariate and multivariate Cox analysis of
(hsa)microRNA alone or in combination with serum CA 19.9 for identification of pancreatic
cancer (n = 198) compared to healthy subjects (n = 153) and patients with chronic pancreatitis
(n = 31).

| | | | | | Multivariate Cox | |
|---|---|---|---|---|---|---|
| | Univariate Cox | | Multivariate Cox | | analysis + | |
| | analysis | | analysis | | CA 19.9 OR | |
| miRNA | OR (95% CI) | P-value | OR (95% CI) | P-value | (95% CI) | P-value |
| (CA 19.9) | | | | | 1.01 (1.01-1.02) | 0.001 |
| | | | | | 1.01 (1.01-1.02)* | 0.001 |
| Let7b | 1.17 (0.88-1.66) | 2.94e−01 | | | | |
| miR-16 | 1.41 (1.21-1.61) | 8.60e−08 | 1.53 (1.08-2.22) | 0.020 | 2.02 (1.16-3.61) | 0.01 |
| | | | 1.51 (1.08-2.17)* | 0.019 | 2.60 (1.49-4.68)* | 0.001 |
| miR-18a | 1.29 (1.18-1.42) | 1.58e−07 | 1.16 (0.98-1.37) | 0.09 | | |
| miR-20a | 1.24 (1.09-1.42) | 1.87e−03 | 0.63 (0.40-0.94) | 0.037 | 0.31 (0.13-0.69) | 0.01 |
| | | | 0.64 (0.42-0.93)* | 0.032 | 0.34 (0.15-0.74)* | 0.01 |
| miR-21 | 1.04 (0.90-1.20) | 6.18e−01 | | | | |
| miR-24 | 1.46 (1.25-1.73) | 5.73e−06 | 1.86 (1.13-3.13) | 0.016 | | |
| | | | 1.74 (1.10-2.79)* | 0.018 | | |
| miR-25 | 1.14 (1.02-1.30) | 2.52e−02 | 0.71 (0.54-0.94) | 0.012 | 0.63 (0.37-1.03) | 0.066 |
| | | | 0.69 (0.52-0.91)* | 0.008 | 0.57 (0.33-0.95)* | 0.031 |
| miR-26a | 1.34 (1.18-1.53) | 9.30e−06 | | | | |
| miR-26b | 1.15 (1.05-1.27) | 3.95e−03 | | | 1.16 (0.95-1.42) | 0.141 |
| | | | | | 1.19 (0.97-1.45)* | 0.091 |
| miR-27a | 1.97 (1.62-2.46) | 8.74e−08 | 1.39 (1.00-1.97) | 0.055 | 1.64 (1.07-2.51) | 0.022 |
| | | | 1.29 (0.93-1.82)* | 0.141 | 1.73 (1.14-2.65)* | 0.011 |
| miR-29c | 1.16 (1.03-1.32) | 2.28e−02 | 0.80 (0.64-1.05) | 0.07 | | |
| | | | 0.82 (0.66-1.05)* | 0.086 | | |
| miR-30a.5p | 1.22 (1.05-1.43) | 1.00e−02 | 1.97 (1.26-3.16) | 0.004 | 1.85 (1.02-3.54) | 0.048 |
| | | | 1.73 (1.12-2.75)* | 0.018 | 2.03 (1.12-3.87)* | 0.023 |
| miR-30e.3p | 1.35 (1.17-1.58) | 9.06e−05 | | | | |
| miR-99a | 0.85 (0.77-0.93) | 3.99e−04 | 0.94 (0.86-1.02) | 0.134 | 0.87 (0.76-0.99) | 0.037 |
| miR-106a | 1.11 (1.02-1.23) | 2.38e−02 | | | | |
| miR-148a | 0.98 (0.88-1.09) | 7.20e−01 | | | | |
| miR-155 | 1.10 (0.97-1.24) | 1.42e−01 | | | | |
| miR-181a | 1.17 (1.06-1.29) | 2.14e−03 | | | | |
| miR-181b | NA | NA | | | | |
| miR-185 | 1.17 (1.06-1.30) | 1.72e−03 | | | | |
| miR-191 | 1.35 (1.17-1.58) | 6.78e−05 | | | | |
| miR-195 | 1.16 (1.06-1.28) | 2.56e−03 | | | 0.70 (0.49-0.98)* | 0.042 |
| miR-196a | 1.08 (0.97-1.21) | 1.60e−01 | | | | |
| miR-210 | 0.93 (0.84-1.02) | 1.46e−01 | | | | |
| miR-212 | 0.99 (0.91-1.06) | 6.94e−01 | | | | |
| miR-320 | 1.11 (0.94-1.32) | 2.29e−01 | | | | |
| miR-323.3p | 1.35 (1.18-1.57) | 4.17e−05 | 1.20 (0.98-1.48)* | 0.086 | | |
| miR-345 | 1.18 (1.05-1.33) | 5.48e−03 | 0.82 (0.64-1.07) | 0.123 | | |
| miR-483.5p | 0.57 (0.49-0.65) | 5.526e−16 | 0.52 (0.43-0.62) | 0.001 | 0.61 (0.47-0.77) | 0.001 |
| | | | 0.49 (0.41-0.58)* | 0.001 | 0.58 (0.45-0.73)* | 0.001 |
| miR-485.3p | 1.12 (1.03-1.21) | 6.73e−03 | 1.09 (1.00-1.18) | 0.061 | | |
| miR-590.5p | 1.22 (1.09-1.38) | 8.69e−04 | 1.23 (0.95-1.61) | 0.129 | | |
| miR-618 | 0.91 (0.83-0.99) | 2.72e−02 | 0.90 (0.82-0.98) | 0.013 | 0.84 (0.74-0.95) | 0.008 |
| miR-638 | 1.05 (0.96-1.14) | 2.72e−01 | | | | |
| miR-645 | 0.96 (0.86-1.07) | 4.55e−01 | | | | |

Italics: miRNAs selected based on results from the literature.
*Only miRNAs with number of missing results <80, and imputation of missing results in each miRNA with 0.95 quantile.

TABLE 12
Univariate and multivariate Cox analysis of (hsa)microRNA alone or in
combination with serum CA 19.9 for identification of pancreatic cancer (n = 198)
compared to healthy subjects (n = 153) in ‘training study’ (example 3—serum).

| | | | | | Multivariate Cox | |
|---|---|---|---|---|---|---|
| | Univariate Cox | | Multivariate Cox | | analysis + | |
| | analysis | | analysis | | CA 19.9 OR | |
| miRNA | OR (95% CI) | P-value | OR (95% CI) | P-value | (95% CI) | P-Value |
| (CA 19.9) | | | | | 1.02 (1.01-1.03) | 0.002 |
| | | | | | 1.02 (1.01-1.03)* | 0.002 |
| Let7b | 1.17 (0.88-1.66) | 2.94e−01 | | | | |
| miR-16 | 1.81 (1.53-2.17) | 2.98e−11 | 7.11 (3.63-15.33) | 0.001 | 20.31 (7.09-76.93) | 0.001 |
| | | | 6.83 (3.51-14.57)* | 0.001 | 18.56 (6.53-69.49)* | 0.001 |
| miR-18a | 1.37 (1.23-1.53) | 1.32e−08 | 1.16 (0.98-1.37) | 0.090 | | |
| | | | 1.13 (0.96-1.33)* | 0.148 | | |
| miR-20a | 1.38 (1.19-1.63) | 6.30e−05 | 0.29 (0.14-0.56) | 0.001 | 0.09 (0.02-0.27) | 0.001 |
| | | | 0.25 (0.12-0.51)* | 0.001 | 0.09 (0.02-0.27)* | 0.001 |
| miR-21 | 1.12 (0.96-1.31) | 1.61e−01 | | | | |
| miR-24 | 1.70 (1.41-2.08) | 9.66e−08 | 3.41 (1.65-7.54) | 0.001 | 19.03 (5.17-89.02) | 0.001 |
| | | | 3.98 (1.85-9.05)* | 0.001 | 14.52 (4.30-60.75)* | 0.001 |
| miR-25 | 1.24 (1.09-1.44) | 2.57e−03 | 0.45 (0.28-0.75) | 0.001 | 0.29 (0.12-0.60) | 0.001 |
| | | | 0.54 (0.32-0.99)* | 0.05 | 0.32 (0.14-0.63)* | 0.003 |
| miR-26a | 1.45 (1.26-1.70) | 7.29e−07 | | | | |
| miR-26b | 1.18 (1.07-1.31) | 1.38e−03 | | | | |
| miR-27a | 1.97 (1.62-2.46) | 1.63e−10 | 1.90 (1.13-3.33) | 0.020 | | |
| | | | 1.95 (1.15-3.45)* | 0.017 | | |
| miR-29c | 1.21 (1.07-1.41) | 7.59e−03 | 0.59 (0.40-0.92) | 0.018 | | |
| | | | 0.58 (0.38-0.92)* | 0.023 | | |
| miR-30a.5p | 1.32 (1.12-1.57) | 1.48e−03 | 2.01 (0.98-4.29) | 0.063 | 6.47 (1.89-25.40) | 0.005 |
| | | | 1.82 (0.91-3.81)* | 0.090 | 4.41 (1.42-15.30) | 0.014 |
| miR-30e.3p | 1.44 (1.23-1.72) | 2.32e−05 | 0.78 (0.58-1.10) | 0.131 | 0.32 (0.14-0.68) | 0.004 |
| | | | | | 0.37 (0.17-0.77)* | 0.010 |
| miR-99a | 0.83 (0.76-0.91) | 1.67e−04 | 0.89 (0.80-1.00) | 0.04 | | |
| miR-106a | 1.16 (1.05-1.30) | 4.57e−03 | | | 1.23 (1.04-1.49) | 0.021 |
| miR-148a | 1.00 (0.89-1.12) | 9.92e−01 | | | | |
| miR-155 | 1.06 (0.93-1.20) | 3.80e−01 | | | | |
| miR-181a | 1.18 (1.07-1.32) | 1.49e−03 | | | | |
| miR-181b | NA | NA | | | | |
| miR-185 | 1.27 (1.14-1.43) | 4.81e−05 | | | 1.33 (0.94-1.88) | 0.10 |
| | | | | | 1.35 (0.96-1.90)* | 0.08 |
| miR-191 | 1.46 (1.25-1.73) | 6.14e−06 | 0.69 (0.44-1.07)* | 0.10 | | |
| miR-195 | 1.25 (1.12-1.41) | 7.96e−05 | | | 0.44 (0.26-0.72) | 0.002 |
| | | | | | 0.55 (0.34-0.86)* | 0.01 |
| miR-196a | 1.08 (0.96-1.21) | 2.11e−01 | | | | |
| miR-210 | 0.99 (0.89-1.10) | 8.64e−01 | | | | |
| miR-212 | 0.98 (0.90-1.06) | 5.51e−01 | | | | |
| miR-320 | 1.16 (0.98-1.40) | 1.10e−01 | | | | |
| miR-323.3p | 1.51 (1.29-1.80) | 1.17e−06 | 1.47 (1.10-2.01) | 0.01 | 1.49 (0.92-2.39) | 0.096 |
| | | | 1.43 (1.08-1.94)* | 0.02 | 1.49 (0.95-2.39)* | 0.085 |
| miR-345 | 1.25 (1.10-1.44) | 8.90e−04 | 0.67 (0.46-0.97) | 0.04 | 0.28 (0.12-0.59) | 0.002 |
| | | | 0.65 (0.44-0.94)* | 0.02 | 0.39 (0.19-0.76)* | 0.007 |
| miR-483.5p | 0.53 (0.45-0.61) | 2.890e−16 | 0.39 (0.28-0.50) | 0.001 | 0.33 (0.20-0.49) | 0.001 |
| | | | 0.36 (0.26-0.46)* | 0.001 | 0.36 (0.23-0.52)* | 0.001 |
| miR-485.3p | 1.12 (1.03-1.21) | 6.97e−03 | | | | |
| miR-590.5p | 1.35 (1.19-1.56) | 1.45e−05 | | | | |
| miR-618 | 0.93 (0.85-1.01) | 9.64e−02 | | | | |
| miR-638 | 1.06 (0.97-1.16) | 2.12e−01 | | | | |
| miR-645 | 0.95 (0.84-1.06) | 3.24e−01 | | | | |

Italics: miRNAs selected based on results from the literature.
*Only miRNAs with number of missing results <80, and imputation of missing results in each miRNA with 0.95 quantile.

TABLE 13A
Diagnostic indexes#1 based on miRNAs in the differential diagnosis of patients with
PC from healthy subjects (HS) and patients with chronic pancreatitis (CP) (serum).

| sPANmiRC | | Micro | |
|---|---|---|---|
| Index | Groups | RNA | MicroRNAs in Index |
| Ia | PC vs. | All | +miR-16 + miR-27a + miR-30a.5p − miR-20a − miR- |
| | HS + CP | | 25 − miR-483.5p |
| IIa | PC vs. | NaN <80 | + miR-16 +miR-27a + miR-30a.5p + miR-323.3p − |
| | HS + CP | | miR-20a − miR-29c − miR-483.5p |
| IIIa | PC vs. | All# | +0.43*miR-16 + 0.62*miR-24 + 0.33*miR-27.a + |
| | HS + CP | | 0.68*miR-30a.5p + 0.08*miR-485.3p − 0.46*miR-20a − |
| | | | 0.34*miR-25 − 0.22*miR-29c − 0.06*miR-99a − |
| | | | 0.20*miR-345 − 0.65*miR-483.5p − 0.11*miR-618 |
| IVa | PC vs. | NaN <80# | +0.41*miR-16 + 0.56*miR-24 + 0.25*miR-27.a + |
| | HS + CP | | 0.55*miR-30a.5p + 0.18*miR-323.3p − 0.44*miR-20a − |
| | | | 0.37*miR-25 − 0.20*miR-29c − 0.71*miR-483.5p |
| Va | PC vs. | All | +miR-24 + miR-27a + miR-323.3p − miR-20a − miR- |
| | HS | | 483.5p |
| VIa | PC vs. | NaN <80 | +miR-16 + miR-27a − miR-25 − miR-29c − miR- |
| | HS | | 483.5p |
| VIIa | PC vs. | All# | +1.96*miR-16 + 0.15*miR-18.a + 1.23*miR-24 + |
| | HS | | 0.64*miR-27a + 0.70*miR-30a.5p + 0.39*miR-323.3p − |
| | | | 1.23*miR-20a − 0.80*miR-25 − 0.53*miR-29c − |
| | | | 0.25*miR-30e.3p − 0.11*miR-99a − 0.40*miR-345 − |
| | | | 0.95*miR-483.5p |
| VIIIa | PC vs. | NaN <80# | +1.92*miR-16 + 0.12*miR-18.a + 1.38*miR-24 + |
| | HS | | 0.67*miR-27a + 0.60*miR-30a.5p + 0.36*miR-323.3p − |
| | | | 1.37*miR-20a − 0.61*miR-25 − 0.55*miR-29c − |
| | | | 0.37*miR-191 − 0.44*miR-345 − 1.03*miR-483.5p |

NaN: Only miRNAs with number of missing results <80, and imputation of missing results in each miRNA with 0.95 quantile.
#Optimal index represents the best fit that can be achieved by using all miRNAs of the final multivariate model shown in Tables 10 and 11.

TABLE 13B
Diagnostic indexes#2 (final) based on miRNAs in the differential diagnosis of
patients with PC from healthy subjects (HS) and patients with chronic pancreatitis (CP)
(example 3 - serum).

| | Case | | |
|---|---|---|---|
| sPANmiRC | vs. | | |
| Index | Control | Model | MicroRNAs in Index |
| I | PC vs. | Only miRNAs | +miR-16 + miR-27a + miR-30a.5p + miR-323.3p − |
| | HS + | with missing | miR-20a − miR-29c − miR-483.5p |
| | CP | values <80 | |
| II | PC vs. | Only miRNAs | +0.41*miR-16 + 0.56*miR-24 + 0.25*miR-27.a + |
| | HS + | with missing | 0.55*miR-30a.5p + 0.18*miR-323.3p − 0.44*miR- |
| | CP | values <80^{#} | 20a − 0.37*miR-25 − 0.20*miR-29c − 0.71*miR- |
| | | | 483.5p |
| III | PC vs. | Only miRNA | +miR-16 + miR-27a − miR-25 − miR-29c − miR- |
| | HS | with missing | 483.5p |
| | | values <70 | |
| IV | PC vs. | Only miRNA | +1.92*miR-16 + 0.12*miR-18.a + 1.38*miR-24 + |
| | HS | with missing | 0.67*miR-27a + 0.60*miR-30a.5p + 0.36*miR- |
| | | values <70^{#} | 323.3p − 1.37*miR-20a − 0.61*miR-25 − |
| | | | 0.55*miR-29c − 0.37*miR-191 − 0.44*miR-345 − |
| | | | 1.03*miR-483.5p |

TABLE 14
Performance measures of Diagnostic indexes of Table 13A based on
miRNAs alone and miRNAs combine with serum log(CA 19.9) in
“Discovery study—Fluidigm method” and “Training study”.

| | | | | +CA | +CA | +CA- |
|---|---|---|---|---|---|---|
| sPANmiRC | Alone | Alone | Alone | 19.9 | 19.9 | 19.9 |
| Index | Sensitivity | Specificity | AUC | Sensitivity | Specificity | AUC |
| I | 75/76 | 81/58 | 0.85/ | | | 0.94 |
| | | | 0.70 | | | |
| II | 75/72 | 81/71 | 0.85/ | | | 0.94 |
| | | | 0.73 | | | |
| III | | | 0.88 | | | |
| IV | | | 0.87 | | | |
| V | 77/74 | 82/75 | 0.87/ | | | 0.95 |
| | | | 0.80 | | | |
| VI | 76/75 | 80/41 | 0.85/ | | | 0.95 |
| | | | 0.63 | | | |
| VII | | | 0.95 | | | |
| VIII | | | 0.95 | | | |

Numbers are Training/Discovery

### Items

1. A method for diagnosing if an individual has, or is at risk of developing, pancreatic carcinoma, and/or a method for giving a prognosis for the survival of the individual, said method comprising measuring the level of at least one miRNA in a blood sample obtained from said individual, wherein the at least one miRNA is selected from the group consisting of or comprising:
   a) in a serum sample for diagnosis
      a. miR-26a, miR-26b, miR-27a, miR-29c, miR-30a-5p, miR-106a, miR-195, miR-212, miR-320, miR-323-3p, miR-345, miR-483-5p, miR-485-3p, miR-590-5p, miR-618, miR-638, miR-25, and miR-645; or
      b. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645; or
      c. let-7b, miR-24, miR-25, miR-26a, miR-26b, miR-27a, miR-29c, miR-30a-5p, miR-30e.3p, miR-106a, miR-148a, miR-185, miR-191, miR-195, miR-212, miR-320, miR-323-3p, miR-345, miR-483-5p, miR-485-3p, miR-590-5p, miR-618, miR-638, and miR-645; and/or
      d. miR-16, miR-27a, miR-30a.5p, miR-20a, miR-25 and miR-483.5p; and/or
      e. miR-16, miR-27a, miR-30a.5p, miR-323.3p, miR-20a, miR-29c and miR-483.5p; and/or
      f. miR-16, miR-24, miR-27a, miR-30a.5p, miR-485.3p, miR-20a, miR-25, miR-29c, miR-99a, miR-345, miR-483.5p and miR-618; and/or
      g. miR-16, miR-24, miR-27a, miR-30a.5p, miR-323.3p, miR-20a, miR-25, miR-29c and miR-483.5p; and/or
      h. miR-24, miR-27a, miR-323.3p, miR-20a and miR-483.5p; and/or
      i. miR-16, miR-27a, miR-25, miR-29c and miR-483.5p; and/or
      j. miR-16, miR-18a, miR-24, miR-27a, miR-30a.5p, miR-323.3p, miR-20a, miR-25, miR-29c, miR-30e.3p, miR-99a, miR-345 and miR-483.5p; and/or
      k. miR-16, miR-18a, miR-24, miR-27a, miR-30a.5p, miR-323.3p, miR-20a, miR-25, miR-29c, miR-191, miR-345 and miR-483.5p; and/or
      l. let-7b, miR-16, miR-18a, miR-20a, miR-24, miR-25, miR-26a, miR-26b, miR-27a, miR-29c, miR-30a-5p, miR-30e.3p, miR-99a, miR-106a, miR-181a, miR-185, miR-191, miR-195, miR-323-3p, miR-345, miR-483-5p, miR-485-3p, miR-590-5p and miR-618; or
      m. miR-16, miR-18a, miR-24, miR-26a, miR-26b, miR-27a, miR-30a.5p, miR-30e.3p, miR-99a, miR-106a, miR-323.3p, miR-20a, miR-25, miR-29c, miR-181a, miR-185, miR-191, miR-195, miR-345, miR-483-5p, miR-485-3p and miR-590.5p; or
      n. miR-16, miR-18.a, miR-20a, miR-24, miR-25, miR-27a, miR-29c, miR-30a.5p, miR-191, miR-323.3p, miR-345, and miR-483.5p.
      o. miR-16, miR-20a, miR-25, miR-27a, miR-30a.5p, miR-99a, miR-195, miR-483.5p and miR-618
      p. miR-16, miR-20a, miR-24, miR-25, miR-30a.5p, miR-30e.3p, miR-106a, miR-195, miR-345, and miR-483.5p
      q. miR-16, miR-20a, miR-24, miR-25, miR-27a, miR-29c, miR-30a.5p, miR-30e.3p, miR-185, miR-195, miR-323.3p, miR-345, miR-483.5p; or
   b) in a whole blood sample for diagnosis
      a. miR-30c, miR-31, miR-150, miR-190, miR-20a and miR-196b; and/or
      b. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      c. miR-20a, miR-30c, miR-31, miR-150, miR-190, miR-196b, let-7b, let-7g, miR-9*, miR-19b, miR-23a, miR-24.2*, miR-26b, miR-30b, miR-31*, miR-34a, miR-93, miR-122, miR-126*, miR-128, miR-143, miR-144*, miR-145, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      d. miR-935, miR-885.5p, miR-769.5p, miR-34a, miR-145, miR-31*, miR-31, miR-199b.5p and miR-150; and/or
      e. miR-935, miR-885.5p, miR-769.5p, miR-34a, miR-145, miR-31*, miR-31, miR-199b.5p, miR-150, miR-93, miR-636, miR-582.3p, miR-126* and miR-122; and/or
      f. let-7g, miR-636, miR-769.5p, miR-93, miR-122, miR-508.3p, miR-199b.5p, miR-935, miR-885.5p, miR-34a, miR-223, miR-144*, miR-145, miR-126*; and/or
      g. let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      h. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      i. let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-505, miR-508.3p, miR-539, miR-582.3p, miR-625, miR-628.3p, miR-636, miR-769.5p, miR-885.5p, miR-935 and miR-941; and/or
      j. let-7g, miR-26b, miR-30b, miR-31, miR-34a, miR-122, miR-126*, miR-145, miR-150, miR-223, miR-505, miR-636 and miR-885.5p; and/or
      k. miR-122, miR-34a, miR-145, miR-636, miR-223, miR-26b, miR-885.5p, miR-150, miR-126* and miR-505; and/or
      l. miR-150, miR-30b, miR-145 and miR-223, or
   c) in a plasma sample for diagnosis miR-let7b, miR-10b*, miR-17, miR-19a, miR-19b, miR-20b, miR-24, miR-27a, miR-30d, miR-93, miR-106a, miR-126, miR-126*, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305; or
   d) in a serum sample for prognosis of miR-19b, miR-27a, miR-30b, miR-30e-3p, miR-99b, miR-100, miR-181a, miR-185, miR-331-3p, miR-511, miR-362-3p, miR-758 and miR-1238; or
   e) in a whole blood sample for prognosis miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636,
   wherein the miRNA level, and/or the difference in the miRNA level, of at least one of said miRNAs compared to a control is indicative of said individual having, or being at risk of developing, pancreatic carcinoma and/or giving a prognosis for the survival of said individual.
2. The method according to the preceding items, wherein the blood sample is a whole blood, serum and/or plasma sample.
3. The method according to the preceding items, said method further comprising the step of extracting RNA from a blood sample collected from an individual.
4. The method according to the preceding items, said method further comprising the step of calculating the difference in level of at least two miRNAs.
5. The method according to the preceding items, said method further comprising the step of determining if said individual has, or is at risk of developing, pancreatic carcinoma; such as pancreatic adenocarcinoma and/or ampullary adenocarcinoma.
6. The method according to the preceding items, wherein there is a difference in the expression level of one miRNA as compared to the expression level of another miRNA.
7. The method according to the preceding items, wherein the expression level of at least one miRNA is altered as compared to the expression level in a control sample.
8. The method according to the preceding items, wherein said control sample is obtained from an individual having a normal pancreas and/or chronic pancreatitis.
9. The method according to the preceding items, wherein said pancreatic carcinoma is pancreatic adenocarcinoma.
10. The method according to the preceding items, wherein said pancreatic carcinoma is ampullary adenocarcinoma.
11. The method according to the preceding items, wherein said pancreatic carcinoma is pancreatic adenocarcinoma and/or ampullary adenocarcinoma.
12. The method of the preceding items, wherein the decreased expression level of at least one of the miRNAs of the following group consisting of or comprising: miR-25, miR-26a, miR-26b, miR-27a, miR-30a-5p, miR-195, miR-323-3p, miR-345, miR-485-3p, miR-618, and miR-645 is measured.
13. The method of the preceding items, wherein the increased expression level of at least one of the miRNAs of the following group consisting of or comprising: miR-29c, miR-106a, miR-212, miR-320, miR-483-5p, miR-590-5p, and miR-638 is measured.
14. The prognostic method according to the preceding items, wherein said sample is a serum sample and at least one miRNA comprises or consists of miR-19b, miR-27a, miR-30b, miR-30e-3p, miR-99b, miR-100, miR-181a, miR-185, miR-331-3p, miR-511, miR-362-3p, miR-758 and miR-1238.
15. The method of the preceding items, wherein the decreased expression level of at least one of the miRNAs following group consisting of or comprising miR-27a, mir-30b, miR-100, miR-181a, miR-185, miR-331-3p, miR-511, miR-19b, and miR-99b is measured.
16. The method of the preceding items, wherein the increased expression level of at least one of the miR following group consisting of or comprising miR-30e-3p, miR-362-3p, and miR-758 is measured.
17. The diagnostic method according to the preceding items, wherein the decreased expression level of at least one of the miRNAs of the following group consisting of or comprising miR-20a, miR-31, miR-150, miR-190, mir-196b, let-7b, let-7g, miR-9*, miR-19b, miR-23a, miR-24.2*, miR-31, miR-31*, miR-93, miR-143, miR-144*, miR-342.5p, miR-345, miR-362.3p, miR-374b*, miR-508.3p, miR-539, miR-628.3p, miR-636 and miR-935 is measured, and said decreased expression corresponds to risk of having pancreatic cancer.
18. The diagnostic method according to the preceding items, wherein the increased expression level of at least one of the miRNA of the following group consisting of or comprising miR-30c, miR-26b, miR-30b, miR-34a, miR-122, miR-126*, miR-128, miR-145, miR-186, miR-199b.5p, miR-223, miR-223*, miR-505, miR-582.3p, miR-625, miR-636, miR-769.5p, miR-885.5p and miR-941 is measured, and said increased expression corresponds to risk of having pancreatic cancer.
19. The prognostic method according to the preceding items, wherein the sample is a whole blood sample and at least one miRNA is selected from the group consisting of or comprising miR-1, miR-23b, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636.
20. The method according to item 19, wherein the decreased expression level of at least one miRNA from the group consisting of or comprising miR-1, miR-150, miR-324-3p, miR-326, miR-370, miR-874, miR-875-3p and miR-484 is measured.
21. The method according to item 19, wherein the increased expression level of at least one miRNA from the group consisting of or comprising miR-27a, miR-296-3p, miR-450a, miR-450b-5p, miR-451, miR-574-3p, miR-23b and miR-636 is measured.
22. The method of any of the preceding items, wherein the level of CA 19-9 in blood is also measured.
23. The method according to the preceding items, wherein the expression level of at least 2 miRNA is measured, such as 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRs, such as 21 miRs, for example 22 miRs, such as 23 miRs, for example 24 miRs, such as 25 miRs, for example 26 miRs, such as 27 miRs, for example 28 miRs, such as 29 miRs, for example 30 miRs, such as 31 miRs, for example 32 miRs, such as 33 miRs, for example 34 miRs, such as 35 miRs, for example 36 miRs, such as 37 miRs, for example 38 miRs, such as 39 miRs, for example 40 miRs.
24. The method according to any of the preceding items, wherein said method comprises the step of obtaining prediction probabilities of between 0-1 for said sample.
25. The method according to the preceding items, wherein said method is used in combination with at least one additional diagnostic or prognostic method.
26. The method according to item 25, wherein said at least one additional diagnostic method is selected from the group consisting of CT (X-ray computed tomography), MRI (magnetic resonance imaging), Scintillation counting, Blood sample analysis, Ultrasound imaging, Cytology, Histology and Assessment of risk factors.
27. The method according to any of the preceding items, wherein the expression level of the at least one miRNA is determined by the microarray technique, by the quantitative polymerase chain reaction (QPCR) technique, by the northern blot technique, or by Nuclease protection assay.
28. A miRNA classifier for characterising a sample obtained from an individual, wherein said miRNA classifier comprises or consists of one or more miRNAs selected from the group consisting of
   a. in a serum sample for diagnosis,
      i. miR-25, miR-26a, miR-26b, miR-27a, miR-29c, miR-30a-5p, miR-106a, miR-195, miR-212, miR-320, miR-323-3p, miR-345, miR-483-5p, miR-485-3p, miR-590-5p, miR-618, miR-638 and miR-645; and/or
      ii. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645; and/or
      iii. let-7b, miR-24, miR-25, miR-26a, miR-26b, miR-27a, miR-29c, miR-30a-5p, miR-30e.3p, miR-106a, miR-148a, miR-185, miR-191, miR-195, miR-212, miR-320, miR-323-3p, miR-345, miR-483-5p, miR-485-3p, miR-590-5p, miR-618, miR-638, and miR-645; and/or
      iv. miR-16, miR-27a, miR-30a.5p, miR-20a, miR-25 and miR-483.5p; and/or
      v. miR-16, miR-27a, miR-30a.5p, miR-323.3p, miR-20a, miR-29c and miR-483.5p; and/or
      vi. miR-16, miR-24, miR-27a, miR-30a.5p, miR-485.3p, miR-20a, miR-25, miR-29c, miR-99a, miR-345, miR-483.5p and miR-618; and/or
      vii. miR-16, miR-24, miR-27a, miR-30a.5p, miR-323.3p, miR-20a, miR-25, miR-29c and miR-483.5p; and/or
      viii. miR-24, miR-27a, miR-323.3p, miR-20a and miR-483.5p; and/or
      ix. miR-16, miR-27a, miR-25, miR-29c and miR-483.5p; and/or
      x. miR-16, miR-18a, miR-24, miR-27a, miR-30a.5p, miR-323.3p, miR-20a, miR-25, miR-29c, miR-30e.3p, miR-99a, miR-345 and miR-483.5p; and/or
      xi. miR-16, miR-18a, miR-24, miR-27a, miR-30a.5p, miR-323.3p, miR-20a, miR-25, miR-29c, miR-191, miR-345 and miR-483.5p; and/or
      xii. let-7b, miR-16, miR-18a, miR-20a, miR-24, miR-25, miR-26a, miR-26b, miR-27a, miR-29c, miR-30a-5p, miR-30e.3p, miR-99a, miR-106a, miR-181a, miR-185, miR-191, miR-195, miR-323-3p, miR-345, miR-483-5p, miR-485-3p, miR-590-5p and miR-618; and/or
      xiii. miR-16, miR-18a, miR-24, miR-26a, miR-26b, miR-27a, miR-30a.5p, miR-30e.3p, miR-99a, miR-106a, miR-323.3p, miR-20a, miR-25, miR-29c, miR-181a, miR-185, miR-191, miR-195, miR-345, miR-483-5p, miR-485-3p and miR-590.5p; and/or
      xiv. miR-16, miR-18.a, miR-20a, miR-24, miR-25, miR-27a, miR-29c, miR-30a.5p, miR-191, miR-323.3p, miR-345, and miR-483.5p; and/or
      xv. miR-16, miR-20a, miR-25, miR-27a, miR-30a.5p, miR-99a, miR-195, miR-483.5p and miR-618; and/or
      xvi. miR-16, miR-20a, miR-24, miR-25, miR-30a.5p, miR-30e.3p, miR-106a, miR-195, miR-345, and miR-483.5p; and/or
      xvii. miR-16, miR-20a, miR-24, miR-25, miR-27a, miR-29c, miR-30a.5p, miR-30e.3p, miR-185, miR-195, miR-323.3p, miR-345, miR-483.5p, or
   b. in a whole blood sample for diagnosis
      i. miR-30c, miR-31, miR-150, miR-190, miR-20a and miR-196b; and/or
      ii. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      iii. miR-20a, miR-30c, miR-31, miR-150, miR-190, miR-196b, let-7b, let-7g, miR-9*, miR-19b, miR-23a, miR-24.2*, miR-26b, miR-30b, miR-31*, miR-34a, miR-93, miR-122, miR-126*, miR-128, miR-143, miR-144*, miR-145, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      iv. miR-935, miR-885.5p, miR-769.5p, miR-34a, miR-145, miR-31*, miR-31, miR-199b.5p and miR-150; and/or
      v. miR-935, miR-885.5p, miR-769.5p, miR-34a, miR-145, miR-31*, miR-31, miR-199b.5p, miR-150, miR-93, miR-636, miR-582.3p, miR-126* and miR-122; and/or
      vi. let-7g, miR-636, miR-769.5p, miR-93, miR-122, miR-508.3p, miR-199b.5p, miR-935, miR-885.5p, miR-34a, miR-223, miR-144*, miR-145, miR-126*; and/or
      vii. let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      viii. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      ix. let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-505, miR-508.3p, miR-539, miR-582.3p, miR-625, miR-628.3p, miR-636, miR-769.5p, miR-885.5p, miR-935 and miR-941; and/or
      x. let-7g, miR-26b, miR-30b, miR-31, miR-34a, miR-122, miR-126*, miR-145, miR-150, miR-223, miR-505, miR-636 and miR-885.5p; and/or
      xi. miR-122, miR-34a, miR-145, miR-636, miR-223, miR-26b, miR-885.5p, miR-150, miR-126* and miR-505; and/or
      xii. miR-150, miR-30b, miR-145 and miR-223; or
   c. in a plasma sample for diagnosis miR-let7b, miR-10b, miR-17, miR-19a, miR-19b, miR-20b, miR-24, miR-27a, miR-30d, miR-93, miR-106a, miR-126, miR-126, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305;
   and distinguishes the combined class of patients with pancreatic carcinoma and ampullary adenocarcinoma from the combined class of individuals with normal pancreas and patients with chronic pancreatitis, wherein said distinction is given as a prediction probability for said sample of belonging to either class, said probability being a number falling in the range of from 0 to 1.
29. A miRNA classifier for predicting with an adequate sensitivity and specificity if a given sample of unknown prognosis has a certain probability of being associated with a specific predicted survival, wherein said miRNA classifier comprises or consists of one or more miRNAs selected from the group consisting of
   a. in a serum sample for prognosis of miR-19b, miR-27a, miR-30b, miR-30e-3p, miR-99b, miR-100, miR-181a, miR-185, miR-331-3p, miR-511, miR-362-3p, miR-758 and miR-1238;
   b. in a whole blood sample for prognosis miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636.
30. The method according to any of items 1 to 27, further comprising use of the miRNA classifier according to any of items 28-29.
31. The miRNA classifier according to any of the preceding items, wherein the sensitivity is at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.
32. The miRNA classifier according to any of the preceding items, wherein the accuracy is at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.
33. The miRNA classifier according to any of the preceding items, wherein the prediction probability of a sample for belonging to a certain class is a number falling in the range of from 0 to 1, such as from 0.0 to 0.1, for example 0.1 to 0.2, such as 0.2 to 0.3, for example 0.3 to 0.4, such as 0.4 to 0.49, for example 0.5, such as 0.51 to 0.6, for example 0.6 to 0.7, such as 0.7 to 0.8, for example 0.8 to 0.9, such as 0.9 to 1.0.
34. The miRNA classifier according to any of the preceding items, wherein the positive predictive value is at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.
35. A model for predicting the diagnosis or prognosis of an individual, comprising
   i) providing a set of input data to the miRNA classifier according to any of items 28-29, and
   ii) determining if said individual has a condition selected from the combined class of pancreatic adenocarcinoma and ampullary adenocarcinoma and the combined class of normal pancreas and chronic pancreatitis.
36. A device for measuring the expression level of at least one miRNA in a sample, wherein said device consists of at least one probe or probe set for at least one miRNA selected from the group consisting of any of groups a), b), c), d) and e) according to claim 1, wherein said device is used for characterising a sample.
37. The device according to item 36, wherein said device may be used for distinguishing between patients with pancreatic carcinoma and individuals with normal pancreas or is used in a method for estimating the probability for a patient with pancreatic cancer of surviving for a certain time period, said method comprising measuring the expression level of at least one miRNA in a sample obtained from said individual.
38. The device according to item 36, wherein said device may be used for distinguishing between patients with pancreatic carcinoma and individuals with normal pancreas.
39. The device according to item 36, wherein said device may be used for distinguishing between patients with pancreatic carcinoma and patients with chronic pancreatitis.
40. The device according to item 36, wherein said device may be used for distinguishing between the combined class of patients with pancreatic carcinoma and ampullary adenocarcinoma from the combined class of individuals with normal pancreas and patients with chronic pancreatitis.
41. The device according to item 36, wherein said device may be used with the miRNA classifier according to the preceding items, to classify a sample into either of the combined class of patients with pancreatic carcinoma and ampullary adenocarcinoma and the combined class of individuals with normal pancreas and patients with chronic pancreatitis.
42. The device according to item 36, wherein said device is used in a method for estimating the probability for a patient with pancreatic cancer of surviving for a certain time period, said method comprising measuring the expression level of at least one miRNA in a sample obtained from said individual
43. The device according to items 36-42, wherein said device is a microarray chip.
44. The device according to item 43, wherein said device is a microarray chip comprising DNA probes.
45. The device according to item 43, wherein said device is a microarray chip comprising antisense miRNA probes.
46. The device according to items 36-42, wherein said device is a QPCR Microfluidic Card.
47. The device according to items 36-42, wherein said device comprises QPCR tubes, QPCR tubes in a strip or a QPCR plate.
48. The device according to items 36-42, wherein said device comprises probes on a solid support.
49. The device according to items 36-42, wherein said device comprises probes on at least one bead.
50. The device according to items 36-42, wherein said device comprises probes in liquid form in a tube.
51. A kit-of-parts comprising at least one of the devices of item 36-50, and at least one additional component.
52. The kit according to item 51, wherein said additional component comprises means for extracting RNA, such as miRNA, from a sample.
53. The kit according to item 51, wherein said additional component comprises reagents for performing microarray analysis.
54. The kit according to item 51, wherein said additional component comprises reagents for performing QPCR analysis.
55. The kit according to item 51, wherein said additional component is the computer program product according to item.
56. The kit according to item 51, wherein said additional component is instructions for use of the device.
57. A system for determining the presence of pancreatic carcinoma in an individual and/or for predicting the prognosis for a patient with pancreatic cancer said system comprising means for analysing the expression level of at least one miRNA in a sample obtained from an individual, wherein said at least one miRNA is selected from the group consisting of
   a. in a serum sample for diagnosis
      i. miR-25, miR-26a, miR-26b, miR-27a, miR-29c, miR-30a-5p, miR-106a, miR-195, miR-212, miR-320, miR-323-3p, miR-345, miR-483-5p, miR-485-3p, miR-590-5p, miR-618, miR-638, and miR-645; and/or
      ii. let-7b, miR-16, miR-18a, miR-20a, miR-21, miR-24, miR-25, miR-26a, miR-26b, miR-27a, miR-29c, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-323-3p, miR-345, miR-483-5p, miR-485-3p, miR-590-5p, miR-618, miR-638, and miR-645; and/or
      iii. let-7b, miR-24, miR-25, miR-26a, miR-26b, miR-27a, miR-29c, miR-30a-5p, miR-30e.3p, miR-106a, miR-148a, miR-185, miR-191, miR-195, miR-212, miR-320, miR-323-3p, miR-345, miR-483-5p, miR-485-3p, miR-590-5p, miR-618, miR-638, and miR-645; or
   b. in a whole blood sample for diagnosis
      i. miR-20a, miR-30c, miR-31, miR-150, miR-190, and miR-196b; and/or
      ii. let-7b, let-7g, miR-9*, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      iii. miR-20a, miR-30c, miR-31, miR-150, miR-190, miR-196b, let-7b, let-7g, miR-9*, miR-19b, miR-23a, miR-24.2*, miR-26b, miR-30b, miR-31*, miR-34a, miR-93, miR-122, miR-126*, miR-128, miR-143, miR-144*, miR-145, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; and/or
      iv. miR-935, miR-885.5p, miR-769.5p, miR-34a, miR-145, miR-31*, miR-31, miR-199b.5p and miR-150; and/or
      v. miR-935, miR-885.5p, miR-769.5p, miR-34a, miR-145, miR-31*, miR-31, miR-199b.5p, miR-150, miR-93, miR-636, miR-582.3p, miR-126* and miR-122; and/or
      vi. let-7g, miR-636, miR-769.5p, miR-93, miR-122, miR-508.3p, miR-199b.5p, miR-935, miR-885.5p, miR-34a, miR-223, miR-144*, miR-145, miR-126*; and/or vii. let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-145, miR-150, miR-186, miR-199b.5p, miR-223, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941, or
   c. in a plasma sample for diagnosis miR-let7b, miR-10b, miR-17, miR-19a, miR-19b, miR-20b, miR-24, miR-27a, miR-30d, miR-93, miR-106a, miR-126, miR-126, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305;
   d. in a serum sample for prognosis of miR-19b, miR-27a, miR-30b, miR-30e-3p, miR-99b, miR-100, miR-181a, miR-185, miR-331-3p, miR-511, miR-362-3p, miR-758 and miR-1238;
   e. in a whole blood sample for prognosis miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636,
58. A system for performing a diagnosis and/or prognosis on an individual, comprising:
   i) means for analysing the miRNA expression profile of a sample obtained from said individual, and
   ii) means for determining if said individual has a condition selected from pancreatic cancer, pancreatic adenocarcinoma, ampullary adenocarcinoma and chronic pancreatitis and/or means for estimating the probability for a patient with pancreatic cancer of surviving for a certain time period wherein said miRNA expression profile comprises at least one miRNA selected from the group consisting of any of a), b), c), d) and e) according to item 57.
59. A computer program product having a computer readable medium, said computer program product providing a system for predicting the diagnosis of an individual, said computer program product comprising means for carrying out any of the steps of any of the methods according to any of items 57 to 58.
60. A system according to any of items 57 to 59, wherein the data is stored, such as stored in at least one database.

1.-27. (canceled)

28. A method for diagnosing if an individual has, or is at risk of developing, pancreatic cancer, and/or a method for predicting the prognosis for survival of an individual diagnosed with pancreatic cancer, said method comprising determining the expression level of one or more miRNA in a blood sample obtained from said individual, wherein the one or more miRNA comprises one or more miRNAs selected from the group consisting of:
a) wherein said blood sample is a whole blood sample;
i) for diagnosis miR-145, miR-150, miR-223, let-7b, let-7g, miR-9*, miR-18a, miR-19b, miR-23a, miR-24.2*, miR-26b, miR-30b, miR-31, miR-31*, miR-34a, miR-93, miR-122, miR-126*, miR-128, miR-143, miR-144*, miR-186, miR-199b.5p, miR-223*, miR-342.5p, miR-345, miR-362.3p, miR-374b*, miR-505, miR-508.3p, miR-539, miR-582.3p, miR-625, miR-628.3p, miR-636, miR-769.5p, miR-885.5p, miR-935 and miR-941; or
ii) for prognosis miR-1, miR-150, miR-324-3p, miR-326, miR-370, miR-874, miR-875-3p, miR-27a, miR-296-3p, miR-450a, miR-450b-5p, miR-451, miR-574-3p, miR-484, miR-23b and miR-636; or
b) wherein said blood sample is a serum sample;
i) for diagnosis miR-24, miR-25, miR-27a, miR-29c, miR-323-3p, miR-483-5p, let-7b, miR-16, miR-18a, miR-20a, miR-21, miR-26a, miR-26b, miR-30a-5p, miR-30e.3p, miR-99a, miR-106a, miR-148a, miR-155, miR-181a, miR-181b, miR-185, miR-191, miR-195, miR-196a, miR-210, miR-212, miR-320, miR-345, miR-485-3p, miR-590-5p, miR-618, miR-638, and miR-645; or
ii) for prognosis miR-19b, miR-27a, miR-30b, miR-30e-3p, miR-99b, miR-100, miR-181a, miR-185, miR-331-3p, miR-511, miR-362-3p, miR-758 and miR-1238; or
c) wherein said blood sample is a plasma sample;
i) for diagnosis miR-let7b, miR-10b*, miR-17, miR-19a, miR-19b, miR-20b, miR-24, miR-27a, miR-30d, miR-93, miR-106a, miR-126, miR-126*, miR-139-5p, miR-140-3p, miR-140-5p, miR-146b-5p, miR-146a, miR-151-3p, miR-151-5p, miR-152, miR-186, miR-191, miR-197, miR-223, miR-320, miR-320b, miR-323-3p, miR-324-3p, miR-328, miR-331-3p, miR-338-5p, miR-340, miR-345, miR-374a, miR-366a, miR-376c, miR-432, miR-518d, miR-520d-3p, miR-548a, miR-575, miR-590-5p, miR-652, miR-720, miR-885-5p, miR-1225-3p, miR-1260, miR-1274b, and miR-1305,
said diagnostic method further comprising the step of determining if said individual has, or is at risk of developing, pancreatic cancer, and
said prognostic method further comprising the step of determining the overall survival of said individual having pancreatic cancer.

29. The method according to claim 28, wherein said method is for diagnosing if an individual has, or is at risk of developing, pancreatic cancer, and said blood sample is a whole blood sample, and wherein said one or more miRNAs comprises one or more miRNAs selected from the group consisting of: miR-26b, miR-34a, miR-122, miR-126*, miR-145, miR-150, miR-223, miR-505, miR-636 and miR-885.5p.

30. The method according to claim 29, wherein said one or more miRNAs comprises at least miR-150, miR-636, miR-145, and miR-223, and optionally one or more additional miRNAs.

31. The method according to claim 28, wherein said method is for diagnosing if an individual has, or is at risk of developing, pancreatic cancer, and said blood sample is a serum sample, and wherein said one or more miRNAs comprises one or more miRNAs selected from the group consisting of: miR-16, miR-18a, miR-20a, miR-24, miR-25, miR-27a, miR-29c, miR-30a-5p, miR-191, miR-323-3p, miR-345 and miR-483-5p.

32. The method according to claim 31, wherein said one or more miRNAs comprises at least:
a) miR-16, miR-27a, miR-30a.5p, miR-20a, miR-25 and miR-483.5p; or
b) miR-16, miR-27a, miR-30a.5p, miR-20a, miR-29c, miR-323.3p, and miR-483.5p; or
c) miR-16, miR-20a, miR-24, miR-25, miR-27a, miR-29c, miR-30a.5p, miR-323.3p and miR-483.5p; or
d) miR-20a, miR-24, miR-27a, miR-323.3p, and miR-483.5p; or
e) miR-16, miR-25, miR-27a, miR-29c and miR-483.5p;
and optionally one or more additional miRNAs.

33. The method according to claim 28, said method further comprising one or more steps of:
a) providing a blood sample in a container suitable for stabilizing the RNA of the blood sample,
b) extracting RNA from a blood sample collected from an individual,
c) correlating the expression level of said one or more miRNA to the expression level in a control sample, wherein said control sample is obtained from an individual having a normal pancreas and/or chronic pancreatitis,
d) obtaining prediction probabilities of between 0-1 for said blood sample, in order to determine if said sample is classified as pancreatic cancer, normal pancreas or chronic pancreatitis.

34. The method according to claim 33, wherein said container is suitable for stabilizing the RNA of the blood sample at room temperature.

35. The method according to claim 28, wherein the expression level of said one or more miRNA is determined by the microarray technique, by the quantitative polymerase chain reaction (QPCR) technique, by the northern blot technique, or by Nuclease protection assay.

36. The method according to claim 28, wherein said pancreatic cancer is pancreatic adenocarcinoma and/or ampullary adenocarcinoma.

37. The method of any claim 28, wherein said method further comprises determining the level of CA 19-9 in blood, and further correlating said levels to a diagnosis and/or prognosis.

38. A diagnostic index for determining if an individual has, or is at risk of developing, pancreatic cancer, comprising measuring:
a) in a whole blood sample
i) the expression level of miR-150, miR-636, miR-145 and miR-223, and correlating the expression levels with the following formula: miR-150+miR-636−miR-145−miR-223; or
ii) the expression level of miR-122, miR-34a, miR-145, miR-636, miR-223, miR-26b, miR-885.5p, miR-150, miR-126* and miR-505, and correlating the expression levels with the following formula:
6.9275−0.2134×miR-122−0.3560×miR-34a−0.8577×miR-145+1.0043×miR-636−0.6725×miR-223+0.7018×miR-26b−0.3233×miR-885.5p+1.1304×miR-150−0.2204×miR-126*−0.1730×miR-505; or
b) in a serum sample
i) the expression level of miR-16, miR-27a, miR-30a.5p, miR-323.3p, miR-20a, miR-29c and miR-483.5p, and correlating the expression levels with the following formula:
+miR-16+miR-27a+miR-30a.5p+miR-323.3p−miR-20a−miR-29c−miR-483.5p; or
ii) the expression level of miR-16, miR-27a, miR-25, miR-29c and miR-483.5p, and correlating the expression levels with the following formula:
+miR-16+miR-27a−miR-25−miR-29c−miR-483.5p; or
iii) the expression level of miR-16, miR-24, miR-27a, miR-30a.5p, miR-323.3p, miR-20a, miR-25, miR-29c and miR-483.5p, and correlating the expression levels with the following formula:
+0.41×miR-16+0.56×miR-24+0.25×miR-27.a+0.55×miR-30a.5p+0.18×miR-323.3p−0.44×miR-20a−0.37×miR-25−0.20×miR-29c−0.71×miR-483.5p; or
iv) the expression level of miR-16, miR-18a, miR-24, miR-27a, miR-30a.5p, miR-323.3p, miR-20a, miR-25, miR-29c, miR-191, miR-345 and miR-483.5p, and correlating the expression levels with the following formula:
+1.92×miR-16+0.12×miR-18.a+1.38×miR-24+0.67×miR-27a+0.60×miR-30a.5p+0.36×miR-323.3p−1.37×miR-20a−0.61×miR-25−0.55×miR-29c−0.37×miR-191−0.44×miR-345−1.03×miR-483.5p.

39. A device for measuring the expression level of one or more miRNA in a blood sample, wherein said device comprises one or more probes, wherein said probes consist of one or more probes for the miRNAs according to claim 28.

40. The device according to claim 39, wherein said device is selected from the group consisting of a microarray chip; a microarray chip comprising DNA probes; a microarray chip comprising antisense miRNA probes; a QPCR Microfluidic Card; QPCR tubes; QPCR tubes in a strip; a QPCR plate; probes on a solid support; probes on at least one bead; and probes in liquid form in a tube.

41. A kit-of-parts comprising a device according to claim 39 and at least one additional component.

42. The kit-of-parts according to claim 41, wherein said additional component comprises one or more of means for collecting, storing or transporting a blood sample; means for extracting RNA, such as miRNA, from a blood sample; and/or reagents for performing miRNA expression analysis.
